(19) United States
(12) Patent Application Publication
(10) Pub. No.: US 2026/0331219 A1
(43) Pub. Date: Oct. 22, 2026

(54) DYNAMIC MINIMAL RESIDUAL DISEASE DETECTION

(71) Applicant: The Broad Institute, Inc., Cambridge, MA (US)

(72) Inventors: Viktor A Adalsteinsson, Acton, MA (US); Timothy David Blewett, Cambridge, MA (US); Ruolin Liu, Cambridge, MA (US)

(73) Assignee: The Broad Institute, Inc., Cambridge, MA (US)

(21) Appl. No.: 19/458,449

(22) Filed: Jan. 23, 2026

Related U.S. Application Data
(63) Continuation of application No. PCT/US2024/039936, filed on Jul. 26, 2024.
(60) Provisional application No. 63/515,801, filed on Jul. 26, 2023.

(51) Int. Cl. G16B 20/20

(52) U.S. Cl. CPC G16B 20/20

(57) ABSTRACT

The present disclosure relates to systems and methods for detecting circulating tumor DNA (ctDNA) and minimal residual disease in a patient sample. Aspects of the present disclosure relate to use of a classification module to assess whether a patient sample comprises ctDNA and minimal residual disease based, in part, on patient-specific inputs. The present disclosure is also related, at least in part, to a determination of whether a patient has cancer based on an output of the classification module.

### CROSS-REFERENCE TO RELATED APPLICATIONS

This application claims the benefit of and priority to International Application No. PCT/US2024/039936, filed Jul. 26, 2024, which claims the benefit under 35 U.S.C. § 119 (e) of U.S. Provisional Application No. 63/515,801, filed Jul. 26, 2023, entitled “MINOR ALLELE ENRICHMENT SEQUENCING THROUGH RECOGNITION OLIGONUCLEOTIDES,” the entire disclosures of which are hereby incorporated by reference in their entireties.

### GOVERNMENT LICENSE RIGHTS

This invention was made with government support under Grant No. CA221874 awarded by the National Institutes of Health. The government has certain rights in the invention.

### BACKGROUND

Tracing patient-specific tumor mutations in cell-free DNA (cfDNA) for minimal residual-disease (MRD) detection is promising but challenging. When a patient cohort is screened, considerations of sensitivity, specificity, and cost should be accommodated. Efforts to improve sensitivity and specificity remain costly and laborious.

### SUMMARY

Aspects of the present disclosure relate to a system comprising: a classification module implemented in a non-transitory computer-readable storage medium and configured to: classify a sample as positive or negative for circulating tumor DNA based on a dynamic probability score relative to a threshold, the dynamic probability score determined from targeted cell-free DNA sequencing data of the sample using specificity factors, the specificity factors including a number of mutated duplexes in the sample bearing mutations assayed in the sample and a total number of assumed duplexes assayed for mutations in the sample, the dynamic probability score further based on: a first likelihood that the number of mutated duplexes is exclusively derived from tumors; and a second likelihood that the number of mutated duplexes is spontaneous error or mutations that are not cancer derived; and output the classification of the sample.

Aspects of the present disclosure relate to a method comprising: receiving targeted cell-free DNA sequencing data for a sample from a patient, the targeted cell-free DNA sequencing data enriched for DNA duplexes with mutation sites found in a tumor fingerprint; determining a dynamic probability score for a minimal residual disease (MRD) status of the sample based on specificity factors including a number of mutated duplexes in the sample and a total number of assumed duplexes assayed for the mutation sites in the sample by: outputting, via a first likelihood model that uses the number of mutated duplexes and the total number of assumed duplexes assayed for the mutation sites in the sample, a first likelihood that the number of mutated duplexes is exclusively derived from tumors; outputting, via a second likelihood model that uses the number of mutated duplexes and the total number of assumed duplexes assayed for the mutation sites in the sample, a second likelihood that the number of mutated duplexes is spontaneous error or mutations that are not cancer derived; and determining the dynamic probability score based on the first likelihood and the second likelihood; classifying the MRD status of the sample as MRD positive or MRD negative based on the dynamic probability score relative to a threshold; and outputting the classified MRD status.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIG. 1 shows an overview of MAESTRO-Pool (Minor Allele Enriched Sequencing Through Recognition Oligonucleotides) and dynamic MRD (Minimal Residual Disease) calling. The workflow consisted of designing patient-specific tumor fingerprints and pooling them together to form one MAESTRO-Pool MRD assay, which was applied to all samples from all patients. Each patient's own plasma samples (i.e., “matched samples”) were used to detect and quantify MRD while other patients' plasma samples (i.e., “unmatched samples”) were used to assess experimental specificity of the tumor fingerprint. MRD was called using a dynamic model that calculates the probability of MRD detection based on sample-specific attributes, such as observed tumor fraction, tumor fingerprint size, and number of cfDNA molecules.

FIGS. 2A-2E show that dynamic MRD calling enables high specificity and sensitivity as mutations and cfDNA molecules are tracked. FIGS. 2A-2E show theoretical specificity and sensitivity while calling MRD with fixed mutation thresholding or dynamic, probabilistic-based thresholding. The left and center graphs portray the probability of false detection using a fixed background error rate of 1×10⁻⁷ across a range of fingerprint sizes and cfDNA masses (FIG. 2A: 50 ng cfDNA; FIG. 2B: 100 ng cfDNA; FIG. 2C: 250 ng cfDNA; FIG. 2D: 500 ng cfDNA; FIG. 2E: 1000 ng cfDNA). The left graphs show the probability of false detection when using a fixed threshold of ≥2 mutations to call MRD and the center graphs show the probability of false detection when using a dynamic threshold of P≥0.95. The right graphs depict the limit of detection (i.e., tumor fraction 90% powered to detect) for the two MRD calling approaches across the same ranges. All the plots assumed 75 duplexes per site per Ing cfDNA.

FIGS. 3A-3B show MAESTRO-Pool and dynamic MRD calling achieved analytical sensitivity below 1 ppm while verifying high experimental specificity of >98%. FIG. 3A shows MRD results from applying MAESTRO-Pool across all samples from all patients and using either fixed MRD calling with a threshold of ≥2 mutations (left) or dynamic MRD calling with a threshold of P≥0.95 (right). Patients are sorted by validated fingerprint size while samples are sorted by cfDNA mass within each patient. FIG. 3B shows that experimental specificity was quantified for each patient's bespoke MRD test (“patient fingerprint”) using unmatched samples and the tumor fraction range is depicted for samples called MRD positive. Again, the results are split between fixed MRD calling (left) and dynamic MRD calling (right).

FIGS. 4A-4D show MAESTRO enrichment reduced sequencing requirements by median 33-fold. FIG. 4A shows a comparison of duplex variant allele frequency (VAF) between MAESTRO and MRD Tracker for sites in both fingerprints. FIG. 4B shows association between MAESTRO's median VAF enrichment per sample and tumor fraction, which establishes an upper bound for VAF enrichment. FIG. 4C shows an example of mutated duplex downsampling that was used for quantifying the read pairs for mutated duplex saturation at sites in both fingerprints. FIG. 4D shows that reduction in read pairs was required to reach mutated duplex saturation due to MAESTRO enrichment.

FIGS. 5A-5B show MAESTRO and dynamic MRD calling provide a sensitive technique for MRD monitoring and often outperform imaging and clinical exams. The figures show overlaying MRD results with clinical annotations for (FIG. 5A) Pt_966 and (FIG. 5B) Pt_1361. Each graph displays the MRD results using MAESTRO and dynamic MRD calling as the line plot, the detection limit of most commercially available MRD tests at 100 ppm, and significant clinical developments (i.e., surgery and progression) along the top axis. Any samples with dynamic scores between 0.5≤P<0.95 were marked as “borderline” and explicitly annotated with their dynamic probability and specificity scores. Detailed clinical annotations including treatments, imaging results, biopsy results, and paraphrased physician notes are shown below the figures. The patient's status at last clinical follow up is displayed at the end of the timeline.

FIGS. 6A-6B show concordance between fixed and dynamic MRD calling in the TBCRC030 cohort. Dynamic MRD calling was applied to 46 patients from the TBCRC030 study, which previously used fixed MRD calling. FIG. 6A shows corresponding MRD calls (inset) and tumor fractions for the two MRD calling methods. FIG. 6B shows the comparison between the dynamic probability score and tumor fraction (line depicting P≥0.95 threshold).

FIGS. 7A-7B show validation of tumor fingerprints in tumor DNA and an estimation of the limit of detection in plasma. FIG. 7A shows that for MAESTRO and MRD Tracker, each patient's tumor fingerprint was applied to their matched tumor and normal DNA for validation. Only sites exclusively detected in the matched tumor were considered validated. FIG. 7B shows that the validated tumor fingerprint was used to compute the limit of detection (LOD) (e.g., lower limit for ≥90% detection power) for each plasma sample.

FIGS. 8A-8B show sensitivity and specificity when using MAESTRO-Pool with different MRD calling thresholds. FIG. 8A shows evaluation of experimental specificity and sensitivity as a function of the fixed mutation threshold. The left plot depicts experimental specificity using the >70 unmatched samples, while the right plot depicts the number of matched samples called MRD positive. The triangle denotes the specificity or sensitivity at the standard fixed threshold of ≥2 mutations. FIG. 8B similarly shows experimental specificity and sensitivity was evaluated for dynamic MRD calling as a function of the dynamic probability threshold. The triangle denotes the specificity or sensitivity when only using a fixed threshold of ≥2 mutations, while the circle denotes the value using a dynamic threshold of P≥0.95. Notably, several patients have specificities of 1 for all dynamic thresholds as shown by the inset.

FIG. 9 shows generation of experimental specificity scores by comparing the dynamic probability score against the distribution from unmatched samples. FIG. 9 shows the distribution of dynamic probability scores for matched and unmatched samples of each tumor fingerprint. A threshold of P≥0.95 was used to call MRD positive samples, while samples with 0.5≤P<0.95 were considered borderline. An experimental specificity score was calculated for each matched borderline sample by assessing the fraction of unmatched samples with a lower probability score.

FIGS. 10A-10B show concordance between MAESTRO and MRD Tracker results. FIG. 10A shows concordance in tumor fraction and MRD calls (shown in inset) when applying MAESTRO with dynamic MRD calling and MRD Tracker with fixed MRD calling to the same samples. FIG. 10B shows subsetting to samples with discordant MRD calls and checking if the limit of detection of the MRD negative sample was higher than the tumor fraction of the MRD positive sample.

FIGS. 11A-11B show a comparison of MAESTRO MRD results with clinical annotations. The figures show overlaying MRD results with clinical annotations for (FIG. 11A) Pt_1070 and (FIG. 11B) Pt_1478. Each graph displays the MRD results using MAESTRO and dynamic MRD calling as the line plot, the detection limit of most commercially available MRD tests at 100 ppm, and significant clinical developments (i.e., surgery and progression) along the top axis. Any samples with dynamic scores between 0.5≤P<0.95 were marked as “borderline” and explicitly annotated with their dynamic probability and specificity scores. Detailed clinical annotations including treatments, imaging results, biopsy results, and paraphrased physician notes are shown below the figures. The patient's status at last clinical follow up is displayed at the end of the timeline.

FIGS. 12A-12B show a comparison of MAESTRO MRD results with clinical annotations. The figures show overlaying MRD results with clinical annotations for (FIG. 12A) Pt_1452 and (FIG. 12B) Pt_973. Each graph displays the MRD results using MAESTRO and dynamic MRD calling as the line plot, the detection limit of most commercially available MRD tests at 100 ppm, and significant clinical developments (i.e., surgery and progression) along the top axis. Any samples with dynamic scores between 0.5≤P<0.95 were marked as “borderline” and explicitly annotated with their dynamic probability and specificity scores. Detailed clinical annotations including treatments, imaging results, biopsy results, and paraphrased physician notes are shown below the figures. The patient's status at last clinical follow up is displayed at the end of the timeline.

FIGS. 13A-13B show a comparison of MAESTRO MRD results with clinical annotations. The figures show overlaying MRD results with clinical annotations for (FIG. 13A) Pt_1083 and (FIG. 13B) Pt_1367. Each graph displays the MRD results using MAESTRO and dynamic MRD calling as the line plot, the detection limit of most commercially available MRD tests at 100 ppm, and significant clinical developments (i.e., surgery and progression) along the top axis. Any samples with dynamic scores between 0.5≤P<0.95 were marked as “borderline” and explicitly annotated with their dynamic probability and specificity scores. Detailed clinical annotations including treatments, imaging results, biopsy results, and paraphrased physician notes are shown below the figures. The patient's status at last clinical follow up is displayed at the end of the timeline.

FIG. 14 shows a comparison of MAESTRO MRD results with clinical annotations. The figure shows overlaying MRD results with clinical annotations for Pt_1406. Each graph displays the MRD results using MAESTRO and dynamic MRD calling as the line plot, the detection limit of most commercially available MRD tests at 100 ppm, and significant clinical developments (i.e., surgery and progression) along the top axis. Any samples with dynamic scores between 0.5≤P<0.95 were marked as “borderline” and explicitly annotated with their dynamic probability and specificity scores. Detailed clinical annotations including treatments, imaging results, biopsy results, and paraphrased physician notes are shown below the figure. The patient's status at last clinical follow up is displayed at the end of the timeline.

FIGS. 15A-15C show the impact of plasma volume on the estimated limit of detection of MAESTRO. FIG. 15A: cfDNA yield for typical (<10 mL) and large (≥10 mL) plasma volume samples; FIG. 15B: Estimated limit of detection with 95% power (LOD95) for typical and large plasma volume samples; FIG. 15C: Estimated LOD95s for large volume plasma samples compared with simulated LOD95s for their single-blood-tube equivalents (i.e., 4 mL). Each large volume plasma sample was downsampled 50 times and the median LOD95 was selected as the predicted LOD95 shown here.

FIGS. 16A-16E show MAESTRO-Pool results. FIG. 16A shows MAESTRO-Pool results when assuming a uniform background SNV frequency. The associated plasma volumes and T>C background SNV frequencies are depicted above with stars denoting samples with unknown plasma volumes. FIG. 16B shows association between false positive MRD calls in patient-unmatched samples and plasma volume (left) and T>C background SNV frequency (right). P-values were evaluated with one-sided Mann-Whitney U test. FIG. 16C shows the improved dynamic MRD caller uses sample- and context-specific background SNV frequencies measured from the MAESTRO-Pool data. FIG. 16D shows MAESTRO-Pool results with sample- and context-specific background SNV frequency tuning. FIG. 16E shows a comparison of MRD positive calls from the dynamic MRD caller using a uniform background SNV frequency or sample- and context-specific background SNV frequencies.

FIGS. 17A-17C show the impact of larger plasma volume on MRD detection. FIG. 17A shows estimated tumor fractions vs. plasma volumes for MRD positive samples. FIG. 17B shows for large volume (≥10 mL) MRD positive samples, comparing detection power (i.e., probability of detecting observed tumor fraction) and the fraction of MRD positive calls among simulated 4 mL equivalents. FIG. 17C shows estimated tumor fractions and confidence intervals for large volume MRD positive samples and their simulated 4 mL equivalents (sorted by descending tumor fraction). Depicted above are metrics derived from these tumor fraction estimations: the confidence interval ratios (CI upper limit/CI lower limit) for each sample as well as the error percentage for the simulated 4 mL equivalents (|TFx4 mL−TFxFull|/TFxFull×100%).

FIGS. 18A-18D show the association of MAESTRO-Pool ctDNA results with clinical responses. The time course for each glioblastoma patient is displayed, including their plasma ctDNA MRD results (top plot); MRI and neuropathology assessments (middle plot); and treatments (bottom plot). Three patterns of association between ctDNA assay results, radiographic assessment, and pathologic assessment were discerned, in which: FIG. 18A shows that MRD positivity preceded histologic tumor progression, whereas the concurrent radiographic findings were indeterminate (i.e., true progression vs. pseudo-progression). FIG. 18B shows that MRD negativity was associated with a lack of tumor progression based on the analysis of large plasma volumes, despite multiple concurrent post-radiotherapy MRIs that were reported as indeterminate progression. FIG. 18C shows persistent negative MRD in a Lynch syndrome patient who exhibited durable responses to immune checkpoint blockade. FIG. 18D shows radiographic and/or pathologic evidence of tumor progression was present; however, MRD was not detected in the preceding timepoints.

FIG. 19 shows the number of total somatic SNVs detected from whole genome sequencing and the number of SNVs that each patient's MAESTRO and MRD Tracker fingerprint targets.

FIGS. 20A-20B show the correlation between measured duplex depth and plasma volume (FIG. 20A) or cfDNA yield (FIG. 20B).

FIGS. 21A-21B show a comparison of MRD calls between MAESTRO-Pool and MRD Tracker assays. FIG. 21A depicts the dynamic caller's probability score with each assay, with dashed lines indicating the MRD calling threshold (i.e., P≥0.95). The table in FIG. 21A denotes the corresponding MRD calls with each assay. Of the 70 total plasma samples, 66 were available for this analysis because 4 samples from GBM_6 were inadvertently mislabeled as GBM 5. These sample swaps were identified with MAESTRO-Pool but were not reanalyzed with the correct MRD Tracker fingerprint. FIG. 21B shows concordance of tumor fractions between MAESTRO-Pool and MRD Tracker assays.

FIG. 22A shows analytical specificity (top) and sensitivity (bottom) of MAESTRO-Pool using the dynamic MRD caller when assuming a uniform background SNV frequency of 0.1 ppm, with respect to each MAESTRO fingerprint. FIG. 22B shows analytical false positive rate (top) and sensitivity (bottom) of MAESTRO-Pool using the dynamic MRD caller with respect to each patient's plasma samples.

FIG. 23A shows overall background SNV frequencies measured by MAESTRO-Pool vs. MRD Tracker. FIG. 23B shows concordance of measured background SNV frequencies. FIG. 23C shows comparison of background SNV frequencies between MAESTRO-Pool and MRD Tracker across time points.

FIG. 24 shows estimated background SNV frequencies using MAESTRO-Pool on 98 plasma samples from 8 melanoma patients.

FIG. 25A shows context-specific background SNV frequencies measured by MAESTRO-Pool. FIG. 25B shows concordance of context-specific background SNV frequencies between MAESTRO-Pool and MRD Tracker.

FIG. 26A shows comparing dynamic probability scores when using a uniform background SNV frequency of 0.1 ppm vs. sample- and context-specific background SNV frequencies. The panels are separated into patient-matched samples (left) and patient-unmatched samples (right). The dashed lines indicate the dynamic probability score threshold of 0.95 for calling MRD. FIG. 26B shows analytical specificity (top) and sensitivity (bottom) of MAESTRO-Pool using sample- and context-specific SNV frequencies with respect to each MAESTRO fingerprint. FIG. 26C shows analytical false positive rate (top) and sensitivity (bottom) of MAESTRO-Pool using sample- and context-specific SNV frequencies with respect to each patient's plasma samples.

FIG. 27 shows MAESTRO-Pool and clinical results for GBM_32. The time course for patient GBM_32 is displayed, including their: plasma ctDNA MRD results (top plot); MRI and neuropathology assessments (middle plot); and treatments (bottom plot). GBM_32 only had four plasma timepoints available for analysis and less frequent MRIs, complicating a rigorous comparison of the two modalities, although the two post-radiotherapy plasma timepoints (days 147, 161) were MRD positive, whereas the concurrent MRI (day 147) noted stable disease with a small dural based area of enhancement that had increased and likely represented an area of tumor seeding.

FIG. 28 is a diagram depicting a flowchart of an illustrative process 2800 for determining a posterior probability that a patient sample is positive for MRD, according to some embodiments of the technology as described herein.

FIG. 29 is a diagram depicting a flowchart of an illustrative process 2900 for identifying the presence of one or more mutation sites in a patient sample, according to some embodiments of the technology described herein.

FIG. 30 is a diagram depicting a flowchart of an illustrative process 3000 for sequencing tumor genomic DNA from a subject and processing and filtering sequencing data from the tumor genomic DNA, according to some embodiments of the technology described herein.

FIG. 31 is a diagram depicting a flowchart of an illustrative process 3100 for determining whether to associate a mutation site with a patient to create a tumor fingerprint, according to some embodiments of the technology described herein.

FIG. 32 is an illustration of an environment in an example implementation that is operable to employ dynamic MRD classification.

FIG. 33 is an illustration of an environment in another example implementation that is operable to employ dynamic MRD classification.

FIG. 34 depicts an example procedure in which dynamic MRD classification is performed.

FIG. 35 illustrates an example system including various components of an example device that can be implemented as any type of computing device as described and/or utilized with reference to FIGS. 28-34 to implement examples of the techniques described herein.

### DETAILED DESCRIPTION

Minimal residual disease (MRD) refers to a small number of cancer cells that remain in the body after or during cancer treatment. Cancer therapeutics have improved significantly over the past century, but many therapeutics do not completely abolish tumor cells in a patient, often resulting in therapy resistance (i.e., cancer cells adapt to a therapeutic while the therapeutic is being administered by acquiring molecular changes that allow the cancer cells to evade the therapy) and/or tumor recurrence (i.e., an otherwise undetectable tumor returns after treatment). Improved methods of cancer cell detection are needed to provide earlier detection of cancer in a patient, treatment monitoring to ensure that a cancer is not becoming therapy resistant, and monitoring for tumor recurrence.

Accordingly, the present disclosure relates to the development of Dynamic Minimal Residual Disease Detection, a method of determining, with statistical significance, whether a patient's sample is MRD-positive or MRD-negative. In some embodiments, Dynamic Minimal Residual Disease Detection is used to evaluate a patient sample that has been analyzed using MAESTRO (Minor Allele Enriched Sequencing Through Recognition Oligonucleotides), a method to enrich for cancer-specific sequence mutations in DNA found in a patient's blood. MAESTRO is described in US 2023/0203568A1, the entire contents of which is hereby incorporated by reference in its entirety.

Dynamic Minimal Residual Disease Detection addresses a long felt but unmet need for a method of detecting cancer in a patient sample long before a tumor develops, detecting whether cancer cells in a patient are becoming therapy resistant, and detecting whether cancer cells remain in a patient who has received cancer therapy. Dynamic Minimal Residual Disease Detection is also useful for cancer treatment planning. The results of the Dynamic Minimal Residual Disease Detection can inform a physician as to what type of cancer therapy to administer to a patient, whether to intensify treatment, whether to de-escalate treatment, whether to change treatment, and/or whether to stop treatment. Dynamic Minimal Residual Disease Detection has the potential to vastly improve cancer patient outcomes by enabling early detection of cancer in a patient, treatment efficacy monitoring, and tumor recurrence monitoring.

Dynamic Minimal Residual Disease Detection is an improved method of determining whether a patient sample is MRD-positive or MRD-negative. In some embodiments, a patient sample is MRD-positive if mutations associated with a patient's tumor are identified in the patient sample. In some embodiments, a patient sample is MRD-negative if mutations associated with a patient's tumor are not identified in the patient sample. Previous methods of determining whether a patient sample is MRD-positive or MRD-negative use a “fixed” threshold for MRD detection: ≥2 mutated DNA duplexes when assaying approximately 1,000 genome-wide mutations from standard blood volumes (e.g., 1-3×10 cc tubes). Such a method is effective in benchmarking experiments, but it is insufficient for tracking additional mutations and cell-free DNA molecules per patient. Dynamic Minimal Residual Disease Detection uses a probability for MRD detection based on sample-specific attributes against an assumed error rate of 1/10 M. Dynamic Minimal Residual Disease Detection is made more sensitive and specific when patient-specific data (e.g., single nucleotide variants found in sequence reads derived from sequencing a patient's plasma sample) are used as background, rather than an assumed background. In some embodiments, Dynamic Minimal Residual Disease Detection detects MRD at or below low parts per million (ppm). In some embodiments, Dynamic Minimal Residual Disease Detection detects MRD at at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 2.0, at least 2.5, at least 3.0, at least 3.5, at least 4.0, at least 4.5, at least 5.0, at least 5.5, at least 6.0, at least 6.5, at least 7.0, at least 7.5, at least 8.0, at least 8.5, at least 9.0, at least 9.5, or at least 10.0 ppm.

Accordingly, aspects of the present disclosure relate to methods of using Dynamic Minimal Residual Disease Detection to determine whether a patient sample is MRD-positive or MRD-negative.

### Cancer Detection Using Dynamic Minimal Residual Disease Detection

Aspects of the present disclosure relate to methods of determining whether a patient is positive for MRD, which may be indicative of the presence of cancer cells in the patient. As used herein, “cancer” refers to any malignant and/or invasive growth or tumor caused by abnormal cell growth in a subject, including solid tumors, blood cancer, bone marrow or lymphoid cancer, etc.

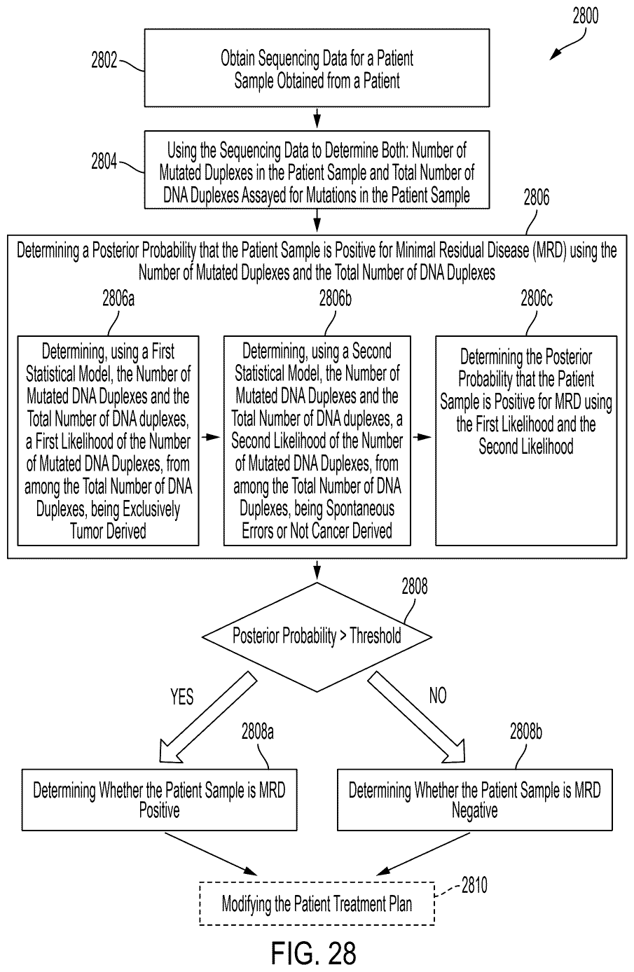

FIG. 28 is a flowchart of an illustrative process 2800 for determining a posterior probability that a patient sample is positive for MRD, using the determined number of mutated duplexes in a patient sample and using the determined number of total duplexes in a patient sample.

Various (e.g., some or all) acts of process 2800 may be implemented using any suitable computing device(s). For example, in some embodiments, one or more acts of the illustrative process 2800 may be implemented in a clinical or laboratory setting. For example, one or more acts of the process 2800 may be implemented on a computing device that is located within the clinical or laboratory setting. In some embodiments, the computing device may directly obtain sequencing data from a sequencing apparatus located within the clinical or laboratory setting.

Additionally or alternatively, one or more acts of the illustrative process 2800 may be implemented in a setting that is remote from a clinical or laboratory setting. For example, the one or more acts of process 2800 may be implemented on a computing device that is located externally from a clinical or laboratory setting. In this case, the computing device may indirectly obtain sequencing data that is generated using a sequencing apparatus located within or external to a clinical or laboratory setting. For example, the expression data may be provided to the computing device via a communication network, such as the Internet or any other suitable network.

It should be appreciated that, in some embodiments, not all acts of process 2800, as illustrated in FIG. 28, may be implemented using one or more computing devices. For example, the act 2810 of modifying a patient's treatment plan may be implemented manually (e.g., by a clinician).

Process 2800 begins at act 2802, wherein sequencing data are obtained for a patient. In some embodiments, sequencing data are obtained from a MAESTRO analysis. In some embodiments, sequencing data are obtained by sequencing DNA duplexes in a patient sample. Examples of sequencing data, sources of sequencing data, and formats of sequencing data are described herein including in the section called “Minor Allele Enriched Sequencing Through Oligonucleotides (MAESTRO).”

As one illustrative example, in some embodiments, the sequencing data may comprise duplex sequencing data. In some embodiments, as described herein, sequencing data comprise data from sequencing DNA duplex molecules captured by allele-specific probes. In some embodiments, sequencing data comprise sequencing reads.

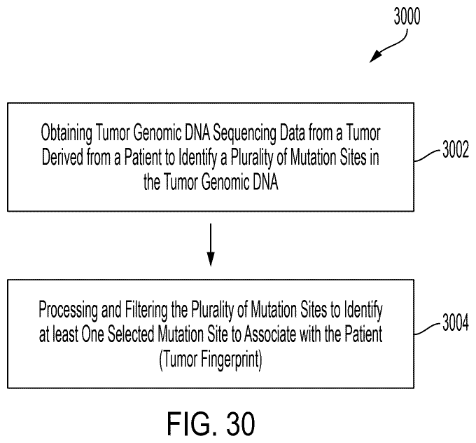

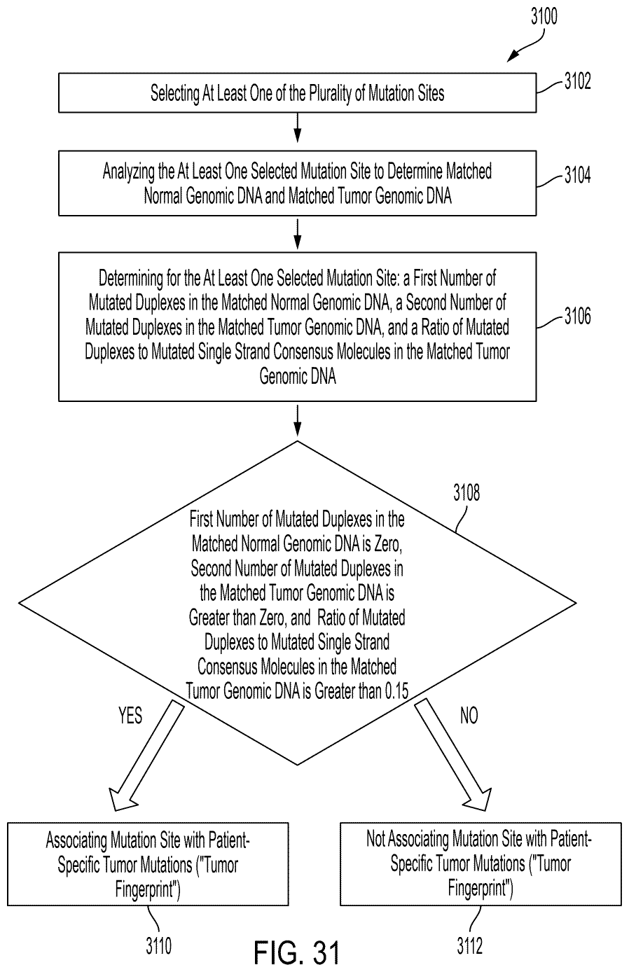

Next, process 2800 proceeds to act 2804, wherein sequencing data are analyzed to determine the number of mutated DNA duplexes in the patient sample and the estimated total number of DNA duplexes assayed for mutations in the patient sample. In some embodiments, the number of mutated DNA duplexes is determined by identifying in the sequencing data mutation sites that are found in the patient's tumor fingerprint. The process of determining a patient's tumor fingerprint is described in FIG. 30 and FIG. 31.

In some embodiments, the estimated total number of DNA duplexes assayed for mutations in a patient sample is estimated. In some embodiments, the total number of estimated DNA duplexes is determined by using probes in the MAESTRO workflow that are not specific to a mutation site found in a patient's tumor fingerprint. The MAESTRO workflow depletes wildtype DNA duplexes and enriches for mutated DNA duplexes. Accordingly, it is difficult to determine the estimated total number of duplexes that were assayed for mutations in the patient sample, thus it is difficult to determine a ratio of mutated DNA duplexes to total DNA duplexes assayed for mutations (tumor fraction). To address this, the numbers of DNA duplexes per locus are measured and used together with the number of mutations in the tumor fingerprint to approximate the total number of DNA duplexes assayed for mutations in a sample. In some embodiments, the average total number of DNA duplexes per locus is multiplied by the number of mutations in the tumor mutation fingerprint to estimate the total number of DNA duplexes assayed for mutations in a patient sample.

In some embodiments, the use of probes that are not specific to a mutation found in a patient's tumor fingerprint are used to enrich for wildtype DNA duplexes and calculate an estimated number of total DNA duplexes per locus. In some embodiments, the estimated total number of DNA duplexes per locus in a patient sample is determined using allele-specific probes that have low binding affinity for the mutation sites found in the patient's tumor fingerprint. Using allele-specific probes that have low binding affinity for the mutation sites found in the patient's tumor fingerprint results in capture of both mutated and wildtype DNA duplexes, which allows for an estimation of the total number of DNA duplexes per locus. The methods described herein to determine the number of mutated DNA duplexes and the number of total DNA duplexes assayed for mutations are used, at least in part, to determine a tumor fraction (TFx). A tumor fraction is a ratio of mutated DNA duplexes to total DNA duplexes assayed for mutations in a patient sample.

Next, process 2800 proceeds to act 2806, wherein the data derived from act 2804 are used to determine a posterior probability that the patient sample is positive for MRD. Act 2806 is separated into three sub-acts. In act 2806a, a first statistical model, the number of mutated DNA duplexes, and the number of total DNA duplexes assayed for mutations are used to determine a first likelihood that the number of mutated DNA duplexes among the estimated total number of DNA duplexes assayed for mutations are exclusively tumor derived. In some embodiments, a first statistical model is a binomial model. The term “binomial model” is known in the art. In some embodiments, a first statistical model is a binomial model that takes the number of mutated duplexes, the estimated total number of duplexes assayed for mutations in a patient sample, and a background mutation frequency as input. In some embodiments, a background mutation frequency is set as the same value for all mutations. In some embodiments, different background mutation frequencies are set for mutations in different mutation contexts. In some embodiments, a background mutation frequency is set using a value empirically measured on a per sample basis. In some embodiments, when the background error rate is set to a value empirically measured on a per sample basis, pooled probe testing is used, as described herein, to determine the background mutation frequency empirically or targeted duplex sequencing is used on the patient sample to determine the value empirically. In some embodiments, the first likelihood is determined as a product of context-specific likelihoods for a discrete set of mutation contexts. In some embodiments, single nucleotide variant abundance in a patient sample is determined to account for background mutation frequency. The term “single nucleotide variant” or “SNV” refers to a change in a DNA sequence that occurs when a single nucleotide is altered in the genome. In some embodiments, SNV frequency is used to calculate a background mutation frequency.

In act 2806b, a second statistical model, the number of mutated DNA duplexes, and the estimated total number of DNA duplexes assayed for mutations are used to determine a second likelihood that the number of mutated DNA duplexes among the estimated total number of DNA duplexes assayed for mutations are spontaneous errors.

After acts 2806a and 2806b are performed, process 2800 proceeds to 2806c, the posterior probability that the patient sample is positive for MRD is determined using the first likelihood and the second likelihood. In some embodiments, a second statistical model is a binomial model. In some embodiments, a second statistical model takes the number of mutated DNA duplexes, the estimated total number of DNA duplexes assayed for mutations in the sample, and a background mutation frequency as input. Methods of determining background mutation frequency are described above under act 2806a. In some embodiments, a second statistical model is a beta-binomial model. The term “beta-binomial model” is known in the art. In some embodiments, the second likelihood is determined as a product of beta-binomial model likelihood, determined for respective contexts using beta-binomial models with context-specific priors set based on context-specific background mutation frequencies. In some embodiments, nucleotide abundance and nucleotide ratio in sequencing data are used to set context-specific priors. In some embodiments, the MAESTRO workflow, described herein, is used to measure sample-specific SNV frequencies. Sample-specific SNV frequencies may be used to determine the likelihood that the mutated DNA duplexes are true mutations derived from a tumor or spontaneous error. The term “spontaneous error” refers to random errors that occur from either a technical artifact or in DNA within a cell. Spontaneous errors can occur naturally by, for example, replication errors in DNA replication. Spontaneous errors determine the background error rate, such as the background single nucleotide variant (SNV) frequency.

Act 2806 provides a posterior probability. Process 2800 proceeds to act 2808, wherein the posterior probability is compared to a pre-determined threshold. Act 2808 is a decision point. If the posterior probability is greater than the pre-determined threshold, then process 2800 proceeds to act 2808a, wherein the patient sample is determined to be MRD positive. If the posterior probability is less than the pre-determined threshold, then process 2800 proceeds to act 2808b, wherein the patient sample is determined to be MRD negative.

Next, process 2800 proceeds to act 2810, wherein the patient's cancer treatment plan is altered based on whether the patient sample is MRD positive or MRD negative.

In some embodiments, if a patient sample is determined to be MRD positive or MRD negative, a patient's cancer treatment plan is altered. In some embodiments, prior to determining the posterior probability that a patient sample is MRD positive, the patient is treated according to an initial treatment plan. In some embodiments, in response to determining that the patient sample is MRD positive, the initial treatment plan is altered to create an updated treatment. In some embodiments, in response to determining that the patient sample is MRD positive, the patient is treated with the updated treatment plan. In some embodiments, an initial treatment plan comprises administering one or more first drugs to the patient at one or more first dosages. In some embodiments, altering the treatment plan comprises increasing one or more of the one or more first dosages to one or more second dosages. In some embodiments, an altered treatment plan comprises administering the one or more first drugs at one or more second dosages. In some embodiments, an altered treatment plan comprises one or more of the one or more first drugs with one or more second drugs. In some embodiments, when a patient sample is determined to be MRD negative, the patient's treatment plan is altered to decrease the dosage of the drugs being administered to the patient or to stop treatment altogether. In some embodiments, a drug is a chemotherapeutic.

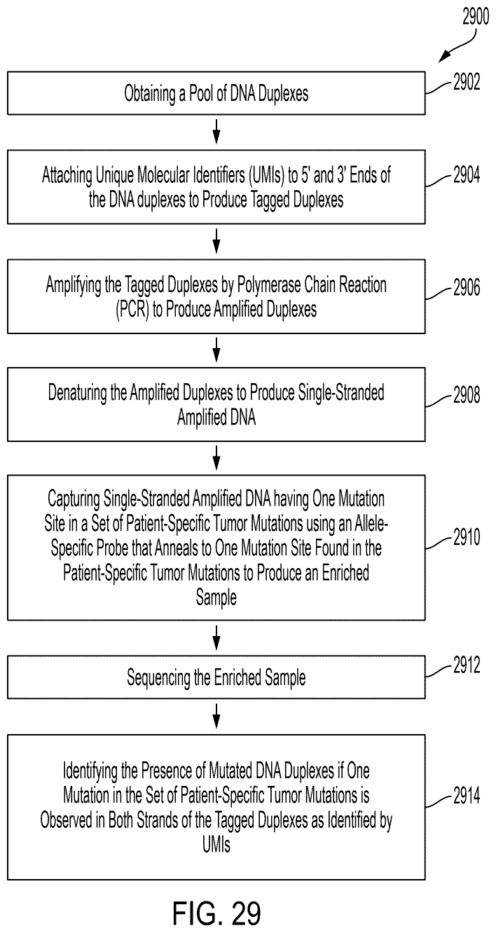

FIG. 29 is a diagram depicting a flowchart of an illustrative process 2900 for identifying the presence of one or more mutation sites in a patient sample. Specifically, FIG. 29 describes the principal steps in the MAESTRO workflow, which is described in detail in the section called “Minor Allele Enriched Sequencing Through Recognition Oligonucleotides (MAESTRO).”

Process 2900 begins at act 2902, wherein a pool of DNA duplexes having, suspected of having, or at risk of having mutation sites found in a tumor fingerprint is obtained. The term “tumor fingerprint” refers to one or more mutations associated with a tumor found in a patient. The process of determining a patient's tumor fingerprint is described in detail in FIG. 30 and FIG. 31.

Next, process 2900 proceeds to act 2904, wherein unique molecular identifiers (UMIs) are attached to the 5′ and 3′ ends of the DNA duplexes in the pool of DNA duplexes to produce tagged duplexes. UMIs, as described herein, are short nucleotide sequences used to uniquely tag each molecule in a patient sample. UMIs are described in detail in the section called “Minor Allele Enriched Sequencing Through Recognition Oligonucleotides (MAESTRO).”

Process 2900 proceeds to act 2906, wherein the tagged DNA duplexes are amplified by polymerase chain reaction (PCR) to produce amplified DNA duplexes.

Process 2900 proceeds to act 2908, wherein the amplified DNA duplexes are denatured to produce single-stranded amplified DNA.

Process 2900 proceeds to act 2910, wherein single-stranded amplified DNA having one or more mutation sites found in the patient's tumor fingerprint are captured using allele-specific probes that anneal to one or more mutation sites found in the tumor fingerprint to produce an enriched sample. Examples of allele-specific probes are described in detail in the section called “MAESTRO Probes.”

In some embodiments, an allele-specific probe is designed to anneal to a mutation site found in a patient's tumor fingerprint. In some embodiments, an allele-specific probe is patient-specific. In some embodiments, a plurality of allele-specific probes are used to capture a plurality of mutation sites found in a patient's tumor fingerprint. In some embodiments, a first set of allele-specific probes are designed to capture a one or more mutation sites found in a first patient's tumor fingerprint. In some embodiments, a second set of allele-specific probes are designed to capture one or more mutation sites found in a second patient's tumor fingerprint. In some embodiments, an N set of allele-specific probes are designed to capture one or more mutation sites found in an N number of patients' tumor fingerprints. In some embodiments, a plurality of sets of allele-specific probes are designed, each set designed to capture one or more mutations in a plurality of patients' tumor fingerprints. In some embodiments, the plurality of sets of allele-specific probes are added to a patient sample derived from a single patient. Using a plurality of sets of allele-specific probes, each set designed to capture one or more mutations in a plurality of patients' tumor fingerprints, in a patient sample derived from a single patient increases the specificity and sensitivity of the set of allele-specific probes that are designed to capture one or more mutations found in the single patient's tumor fingerprint. The sensitivity and specificity are increased because the allele-specific probes that were not designed to capture the one or more mutations found in the single patient's tumor fingerprint serve as internal controls to which the allele-specific probes designed to capture one or more mutations found in the single patient's fingerprint are compared.

In some embodiments, additional probes that have low binding specificity for the mutation sites found in the patient's tumor fingerprint are used to determine the estimated total number of DNA duplexes assayed for mutations in the patient sample.

Process 2900 proceeds to act 2912, wherein the enriched sample is sequenced. In some embodiments, the enriched sample is sequenced using next-generation sequencing (NGS).

Process 2900 proceeds to act 2914, wherein the presence of one or more mutation sites is identified if one or more mutation sites are observed in both strands of the tagged duplexes, as identified by analyzing the UMI sequences.

FIG. 30 is a flowchart of an illustrative process 3000 for sequencing tumor genomic DNA from a subject and processing and filtering sequencing data from the tumor genomic DNA.

Various (e.g., some or all) acts of process 3000 may be implemented using any suitable computing device(s). For example, in some embodiments, one or more acts of the illustrative process 3000 may be implemented in a clinical or laboratory setting. For example, one or more acts of the process 3000 may be implemented on a computing device that is located within the clinical or laboratory setting. In some embodiments, the computing device may directly obtain sequencing data from a sequencing apparatus located within the clinical or laboratory setting.

Additionally or alternatively, one or more acts of the illustrative process 3000 may be implemented in a setting that is remote from a clinical or laboratory setting. For example, the one or more acts of process 3000 may be implemented on a computing device that is located externally from a clinical or laboratory setting. In this case, the computing device may indirectly obtain sequencing data that is generated using a sequencing apparatus located within or external to a clinical or laboratory setting. For example, the expression data may be provided to the computing device via a communication network, such as the Internet or any other suitable network.

Process 3000 begins at act 3002, wherein tumor genomic DNA sequencing data are obtained from a tumor derived from a patient to identify a plurality of mutation sites in the tumor genomic DNA.

Process 3000 proceeds at act 3004, wherein the plurality of mutations sites are processed and filtered to identify at least one selected mutation site to associate with the patient, thus creating a tumor fingerprint. The steps of processing and filtering to identify at least one selected mutation site are described in detail in FIG. 31.

FIG. 31 is a flowchart of an illustrative process 3100 for sequencing tumor genomic DNA from a subject and processing and filtering sequencing data from the tumor genomic DNA.

Various (e.g., some or all) acts of process 3100 may be implemented using any suitable computing device(s). For example, in some embodiments, one or more acts of the illustrative process 3100 may be implemented in a clinical or laboratory setting. For example, one or more acts of the process 3100 may be implemented on a computing device that is located within the clinical or laboratory setting. In some embodiments, the computing device may directly obtain sequencing data from a sequencing apparatus located within the clinical or laboratory setting.

Additionally or alternatively, one or more acts of the illustrative process 3100 may be implemented in a setting that is remote from a clinical or laboratory setting. For example, the one or more acts of process 3100 may be implemented on a computing device that is located externally from a clinical or laboratory setting. In this case, the computing device may indirectly obtain sequencing data that is generated using a sequencing apparatus located within or external to a clinical or laboratory setting. For example, the expression data may be provided to the computing device via a communication network, such as the Internet or any other suitable network.

Process 3100 begins at act 3102, wherein at least one of a plurality of mutation sites identified in FIG. 30 is selected.

Process 3100 proceeds at act 3104, wherein at least one selected mutation site is analyzed to determine matched normal genomic DNA and matched tumor genomic DNA.

Process 3100 proceeds at act 3106, wherein the following is determined for the at least one mutation site: a first number of mutated duplexes in the matched normal genomic DNA, a second number of mutated duplexes in the matched tumor genomic DNA, and a ratio of mutated duplexes (ALT duplexes) and mutated single strand consensus molecules (ALT single strand consensus molecules) in the matched tumor genomic DNA.

Process 3100 proceeds at act 3108, which is a decision point. If the first number of mutated duplexes in the matched normal genomic DNA is zero, the second number of mutated duplexes in the matched tumor genomic DNA is greater than zero, and the ratio of mutated duplexes to mutated single strand consensus molecules in the matched tumor genomic DNA is greater than 0.15, then process 3100 proceeds to act 3110. If the number of mutated duplexes in the matched normal genomic DNA is not zero, the second number of mutated duplexes in the matched tumor genomic DNA is not greater than zero, or the ratio of mutated duplexes to mutated single strand consensus molecules in the matched tumor genomic DNA is not greater than 0.15, then process 3100 proceeds to act 3112.

If process 3100 proceeds to act 3110, then the mutation site is associated with the patient and the mutation site becomes part of the patient's tumor fingerprint. If process 3100 proceeds to act 3112, then the mutation site is not associated with the patient, and the mutation site does not become part of the patient's tumor fingerprint.

The processes described herein are used to determine whether a patient sample is MRD positive or MRD negative. The method described herein was developed as a result of the surprising finding that using a probabilistic analysis to interpret the MAESTRO workflow can increase the sensitivity and the specificity of MAESTRO. In some embodiments, using the methods described herein, MRD is detected between at least 0.10 parts per million (ppm) and at least 10.0 ppm of tumor-derived cell-free DNA. In some embodiments, MRD is detected at at least 0.10, at least 0.15, at least 0.20, at least 0.25, at least 0.30, at least 0.35, at least 0.40, at least 0.45, at least 0.50, at least 0.55, at least 0.60, at least 0.65, at least 0.70, at least 0.75, at least 0.80, at least 0.85, at least 0.90, at least 0.95, or at least 1.00 ppm. In some embodiments, MRD is detected at 0.78 ppm. The specificity of the methods described herein is a significant improvement over other methods that often detect MRD only when the concentration of MRD is 10.0 ppm or greater. In some embodiments, mutation sites derived from a patient's tumor fingerprint are detected using the methods described herein with high specificity. In some embodiments, the specificity is at least 95%, at least 96%, at least 97%, at least 98%, at least 99%, or 100% specificity. In some embodiments, the specificity is 98% or greater.

The methods described herein have multiple advantages over existing methods of identifying MRD in a patient sample.

### Minor Allele Enriched Sequencing Through Recognition Oligonucleotides (MAESTRO)

Aspects of the present disclosure relate to the use of Minor Allele Enriched Sequencing Through Recognition Oligonucleotides (MAESTRO). MAESTRO is described in US 2023/0203568A1, the entire contents of which is hereby incorporated by reference in its entirety.

MAESTRO is a method of identifying the presence of one or more mutation sites, or specific mutations, in a patient sample. An embodiment of the MAESTRO method comprises: (a) obtaining a pool of DNA duplexes having, suspected of having, or at risk of having one or more of the mutations sites in at least one strand of the DNA duplexes, and optionally fragmenting the DNA duplexes; (b) attaching (e.g., ligating) a unique molecular identifier (UMI) (e.g., as part of an adapter molecule) to the 5′ and 3′ ends of the DNA duplexes to produce tagged duplexes, wherein the UMIs are unique to each tagged duplex; (c) amplifying the tagged duplexes by polymerase chain reactions (PCR) to produce amplified duplexes; (d) denaturing the amplified duplexes to produce single-stranded amplified DNA; (e) capturing single-stranded amplified DNA having the specific mutation using an allele-specific probe that anneals to the specific mutation to produce an enriched sample; (f) sequencing the enriched sample; and (g) confirming the presence of the specific mutation if the specific mutation is observed in both strands of the tagged duplex as identified by the UMIs.

In some aspects, the MAESTRO method comprises: (a) obtaining a pool of DNA duplexes comprising a specific mutation in at least one strand and attaching (e.g., ligating) a unique molecular identifier (UMI) to the 5′ and 3′ ends of each strand of the DNA duplexes to produce tagged duplexes, wherein the UMIs are specific to each tagged duplex; (b) amplifying the tagged duplexes by polymerase chain reactions (PCR) to produce amplified duplexes and subsequently denaturing the amplified duplexes to produce single-stranded amplified DNA; (c) capturing single-stranded amplified DNA having the specific mutation using an allele-specific probe that anneals to the specific mutation to produce an enriched sample, and sequencing the enriched sample; and (d) calculating a double-stranded consensus (DSC) to single-stranded consensus (SSC) ratio (DSC to SSC ratio) using the UMIs, and identifying the specific mutation if the DSC to SSC ratio is greater than 0.15.

The terms “specific mutation” and “mutation site” as may be used herein, refer to a change, alteration, or modification to a nucleotide in a nucleic acid as compared to its wild-type sequence (e.g., unmutated, reference sequence), which is targeted by a probe of the disclosure and is of interest. For example, a specific mutation may be known to be associated with a disorder (e.g., disease or condition). As such, evaluating a subject, or sample from a subject (e.g., pool of DNA duplexes) for the presence of a specific mutation, or evaluating the same for identification of any of such specific mutations, may be useful in, without limitation, the diagnosis, treatment, and/or evaluation of a subject. In some embodiments of the disclosure, the identification, and/or presence of a specific mutation is used to indicate the presence of nucleic acids (e.g., DNA, cfDNA) related to a disorder. In some embodiments, the methods of this disclosure use this determination to indicate and/or evaluate a subject for minimal residual disease (MRD).

Without limitation, mutations may include substitutions, insertions, deletions, or any combination of the same. In some embodiments, there is at least one mutation. In some embodiments, there is more than one mutation. In some embodiments, where there is more than one mutation, the mutations are distinct (e.g., not of the same type (e.g., substitutions, insertions, deletions)). In some embodiments, where there is more than one mutation, the mutations are the same (e.g., of the same type (e.g., substitutions, insertions, deletions)). Additionally, in some embodiments, mutations result in a frameshift. In some embodiments, a mutation comprises a single nucleotide polymorphism (SNP). In some embodiments, a mutation is a structural variant. As used herein, a structural variant shall refer to a variation in structure of a chromosome of a subject, such variation can comprise many kinds of variation in the genome of a subject. For example, without limitation, structural variations can include microscopic and submicroscopic alterations, such as deletions, duplications, copy-number variants, insertions, inversions and translocations. In some embodiments, a mutation occurs in one strand of a nucleic acid duplex. In some embodiments, the strand is the plus strand (e.g., ‘+’, sense strand). In some embodiments, the strand is the negative strand (e.g., ‘−’, antisense strand). In some embodiments, a mutation occurs in both strands of a nucleic acid duplex (e.g., ‘+’ and ‘−’ strands). In some embodiments, a mutation is a mutation known to be associated with a cancer. In some embodiments, a cancer is leukemia. In some embodiments, a mutation is known to be related to, or originated in, tumor tissue.

In some embodiments, specific mutations are chosen (e.g., established as targets) based on existing information such as literature presenting lists of known mutations, databases of known mutations, and/or any other sources of known mutations. In some embodiments, specific mutations are chosen from existing information about a subject (e.g., the subject from which the pool of DNA duplexes and/or enriched sample will be obtained). For example, the existing information may be subject history of disease or disorder, or subject history of a specific mutation. In some embodiments, a specific mutation is chosen based on known association with a disease or disorder. In some embodiments, a specific mutation is chosen based on the fact that a subject has, is suspected of having, or has had a disease of which the specific mutation is associated or related. In some embodiments, a specific mutation is chosen based on existing information or sequencing data from a tissue sample of a subject (either presently obtained or obtained in the past). In some embodiments, the tissue sample is tumor tissue.

In some embodiments, DNA duplexes are obtained from a sample. As used in the methods herein, a sample may be any sample from a subject. In some embodiments, a sample is a patient sample. In some embodiments, a patient sample is a biological sample. A biological sample can be from, without limitation, blood, skin, tissue, hair, saliva, bodily fluid, cells, or any other biological component from which the skilled artisan may ascertain, using techniques known and readily available in the art, the parameter being evaluated (e.g., presence or absence of nucleic acids containing a specific mutation or duplexes containing the same). In some embodiments, a sample is a blood sample. In some embodiments, a blood sample contains cell-free DNA (“cfDNA”). In some embodiments, a sample is a plasma sample. In some embodiments, a plasma sample comprises cfDNA.

In some embodiments, a sample is acquired by biopsy. In some embodiments, a biopsy is a liquid biopsy. Liquid biopsies are well-known in the field to the skilled artisan. They are generally known to be liquid or fluid phase biopsies where the sampling and analysis is that of non-solid biological matter from a subject (e.g., bodily fluid, blood, saliva, etc.). A sample from the liquid biopsy is then analyzed for the presence of markers (e.g., specific mutations or nucleic acids and/or duplexes bearing specific mutations or sequences). The component of the fluid may vary depending on the target to be analyzed, for example, circulating tumor cells and/or circulating tumor DNA (ctDNA), circulating endothelial cells, cell-free DNA (cfDNA), and/or cell-free fetal DNA (cffDNA). In some embodiments, a liquid biopsy sample is a blood sample. In some embodiments, a liquid biopsy is of the reproductive cells of a subject (e.g., from eggs or spermatozoa). In some embodiments, cfDNA is targeted by the methods of the disclosure. However, any suitable liquid biopsy may be used with the methods herein as can be determined by the skilled artisan without undue experimentation.

Once the sample is obtained (e.g., acquired), DNA duplexes are analyzed using the sample. The term “DNA duplex,” as may be used herein, refers to an individual double-stranded nucleic acid molecule. As such, the term shall be understood to include genomic DNA (gDNA), germline DNA, cell-free DNA, and other forms of DNA provided the molecule comprises two annealed strands for at least a portion of the nucleic acid molecule. Accordingly, a DNA duplex may refer to an intact DNA molecule comprising an entire genome, portion thereof, or fragments thereof (e.g., after fragmenting, shearing), provided the molecule remains double-stranded for at least a portion of the nucleic acid molecule.

In some embodiments, DNA duplexes are fragmented. This fragmentation breaks apart a nucleic acid into small fragments. In some embodiments, a DNA duplex is fragmented to reduce its size. In some embodiments, a DNA duplex is fragmented to make DNA duplexes more homogenous with respect to the size of DNA duplexes. In some embodiments, a DNA duplex is fragmented to produce fragments of about 50 to about 250 base pairs in length (e.g., about 50 to about, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 base pairs in length). In some embodiments, a DNA duplex is fragmented to produce fragments of about 100 to about 200 base pairs in length. In some embodiments, a DNA duplex is fragmented to produce fragments of about 120 to about 180 base pairs in length. In some embodiments, a DNA duplex is fragmented to produce fragments of about 130 to about 170 base pairs in length. In some embodiments, a DNA duplex is fragmented to produce fragments of about 140 to about 160 base pairs in length. In some embodiments, a DNA duplex is fragmented to produce fragments of about 150 base pairs in length. In some embodiments, a DNA duplex is already fragmented, e.g., cell-free DNA from blood plasma.

Fragmentation may be accomplished, physically (e.g., by sonication or physical force), enzymatically, or chemically. However, all forms of fragmentation inherently damage the strands to break them into smaller portions. Methods of fragmentation are well-known in the art and will be readily appreciated and selected by the skilled artisan. In some embodiments, prior to step (a) a sample has been: (i) fragmented; or (ii) cleaved and tagged (tagmented). In some embodiments, fragmentation is by: (a) physical fragmentation; (b) enzymatic fragmentation; and/or (c) chemical fragmentation. In some embodiments, fragmentation is by physical fragmentation. In some embodiments, physical fragmentation is by nebulization. In some embodiments, physical fragmentation is by acoustic shearing. In some embodiments, physical fragmentation is by needle shearing. In some embodiments, physical fragmentation is by French pressure cell. In some embodiments, physical fragmentation is by sonication. In some embodiments, physical fragmentation is by hydrodynamic shearing. In some embodiments, fragmentation is by enzymatic fragmentation. In some embodiments, enzymatic fragmentation is by nuclease or endonuclease. In some embodiments, enzymatic fragmentation is by DNase I. In some embodiments, enzymatic fragmentation is by restriction endonuclease. In some embodiments, enzymatic fragmentation is by transposase. In some embodiments, fragmentation is by chemical fragmentation. In some embodiments, chemical fragmentation is by heat and divalent metal cation fragmentation.

Once a DNA duplex is fragmented, unique molecular identifiers (UMIs) may be ligated to one or both ends of the DNA duplex as part of a sequencing adapter, which contains sequences to facilitate primer binding and amplification. This process of sequencing preparation is well established in the art, while there are also other ways to append sequencing adapters comprising UMIs. UMIs are tags (e.g., specific sequences), which may be useful in identifying a strand and/or its duplex counterpart (e.g., complementary strand) throughout the remainder of the method and during any post sequencing processing and/or evaluation (e.g., analysis). In some embodiments, UMIs are contained within a sequencing adapter. Use of UMIs is well-known throughout the field. In some embodiments, a UMI is attached to at least a 5′ end of at least one strand of a DNA duplex. In some embodiments, a UMI is attached to both 5′ ends of a DNA duplex. In some embodiments, a UMI is attached to at least a 3′ end of at least one strand of a DNA duplex. In some embodiments, a UMI is attached to both 3′ ends of a DNA duplex. In some embodiments, a UMI is attached to at least each of, a 5′ end of at least one strand of a DNA duplex, and a 3′ end of at least one strand of a DNA duplex. In some embodiments, a UMI is attached to both 5′ and both 3′ ends of a DNA duplex. In some embodiments, UMIs attached to a DNA duplex are identical to each other, but unique to a DNA duplex. In some embodiments, UMIs of a DNA duplex are unique to each other and unique to a DNA duplex. In some embodiments, UMIs are not unique to the DNA duplex, but when evaluated in combination with the start and/or stop sequencing sites, are unique to the DNA duplex. In some embodiments, UMIs are between about 1 nucleotide and about 20 nucleotides in length. In some embodiments, UMIs are between about 3 nucleotides and about 18 nucleotides in length. In some embodiments, UMIs are between about 5 nucleotides and about 16 nucleotides in length. In some embodiments, UMIs are between about 6 nucleotides and about 15 nucleotides in length. In some embodiments, UMIs are between about 8 nucleotides and about 15 nucleotides in length. In some embodiments, UMIs are attached to the DNA duplex by ligation. One of the benefits and features of duplex sequencing is that the association between UMI sequences added to top and bottom strands are known (e.g., are complementary to one another, or provide indication of which sequence comes from the top and bottom strand) so reads from each strand can be paired back to the same original DNA duplex. This knowledge is a component of duplex sequencing. In some embodiments, the UMIs are unique to each duplex. In some other embodiments, there will be DNA duplexes that will share the same UMI sequence. However, the odds that two DNA duplexes will share the same UMI and the same start and stop position in the genome is highly unlikely. With this principle in mind, the sequencing reads can be de-duplicated.

After UMI attachment (e.g., an adapter comprising a UMI), a DNA duplex is amplified to produce amplified duplexes (i.e., a sequencing library, which may be defined as a collection of DNA fragments that have adapters added to facilitate their amplification and sequencing). Any suitable method known to the skilled artisan may be employed, but generally amplification is accomplished by means of polymerase chain reaction (PCR). PCR has been known in the field for a number of decades and is well-documented and the methods and protocols are readily available and will be immediately appreciated by the skilled artisan. In some embodiments, a DNA duplex is amplified by PCR.

Once amplified, an amplified DNA duplex (i.e., the sequencing library) will need to be prepared for capture by the allele-specific probes of the disclosure. In some embodiments, an amplified DNA duplex (i.e., the sequencing library) will be denatured to separate the strands of a DNA duplex, producing single-stranded amplified DNA. Any method suitable as determined by the skilled artisan may be used to denature or separate the strands, for example, without limitation, changing the temperature of the environment of a DNA duplex (e.g., apply heat, reduce temperature), sodium hydroxide (NaOH) treatments, or placing a DNA duplex in a salt rich environment. In some embodiments, a DNA duplex is denatured (e.g., strands separated) by changing the temperature of the environment. In some embodiments, the temperature change is accomplished through the application of heat.

Once the DNA duplexes have been fragmented, have UMIs attached, are amplified, and are denatured, the DNA duplexes can then be enriched for target sequences (e.g., single-stranded amplified DNA harboring (e.g., containing) a specific mutation). The enrichment process may be accomplished by the use of probes. In some embodiments, a probe of the disclosure, is any of the probes as described herein or according to the methods of making a probe as disclosed herein. In some embodiments, a probe is an allele-specific probe. Further embodiments of probes are disclosed hereinbelow. In some embodiments, a probe comprises a sequence complementary to a portion of a single-stranded amplified DNA (e.g., such that it targets and anneals to that sequence (e.g., discriminately binds)), wherein the portion comprises a specific mutation, and a means by which to recover (e.g., capture) or separate the probe from extraneous material (e.g., unbound nucleic acids). For example, a probe may target a sequence as described herein, and comprise biotin. As such, the probe may be recovered exploiting the properties of biotin to bind streptavidin. Once the probes are bound to a single-stranded amplified DNA comprising a specific mutation, they are captured, thus producing an enriched sample. Through this process the sample will comprise a higher concentration of single-stranded amplified DNA comprising a specific mutation, than the original sample (e.g., is enriched for single-stranded amplified DNA comprising a specific mutation). This process of capturing (e.g., enriching for) single-stranded amplified DNA may occur once, or multiple times. In instances where capturing is performed multiple times (e.g., enriching multiple times), capture may be performed on a sample comprising the single-stranded amplified DNA and/or an enriched sample. In some embodiments, capture is performed at least one time. In some embodiments, capture is performed more than one time (e.g., 2, 3, 4, 5, 6, or more). In some embodiments, capture is performed less than 10 times. In some embodiments, capture is performed more than 10 times. In some embodiments, capture is performed more than 100 times. In some embodiments, capture is performed more than 1,000 times.

Additionally, capture may be performed using multiple probes. In some embodiments, more than one probe is used to capture single-stranded amplified DNA. In some embodiments, the multiple probes may be distinct, and target the same specific mutation. In some embodiments, more than one probe is used during capture, which probes are distinct from one another and target different specific mutations. By using different probes that target sequences comprising different (e.g., distinct) specific mutations, the methods of the disclosure can be used to capture (e.g., enrich) DNA duplexes for a set (e.g., panel, plurality) of mutations concurrently (e.g., simultaneously). Each probe may target a specific mutation (or more than one mutation), which is known to be associated with the same disorder, or distinct disorders. In some embodiments, wherein multiple probes are used, each targets a specific mutation (the same, distinct, or combination thereof) wherein all specific mutations are related or known to be associated with a single disorder (e.g., disease). In some embodiments, wherein multiple probes are used, each targets a specific mutation wherein at least one of the specific mutations is related or known to be associated with at least one disorder (e.g., disease) which is distinct from at least one disorder known to be associated with at least one other specific mutation.

In some embodiments, where more than one probe is used, each of the probes targets the same specific mutation targeted by other probes. In some embodiments, where more than one probe is used, at least one of the probes targets a specific mutation distinct from a specific mutation targeted by at least one other probe.

In some embodiments, at least 25 (e.g., 25, 26, 27, 28, 50, 100, or more) distinct probes are used (e.g., target 25 distinct specific mutations). In some embodiments, at least 50 (e.g., 50 or more) distinct probes are used (e.g., target 50 distinct specific mutations). In some embodiments, at least 100 distinct (e.g., 100 or more) probes are used (e.g., target 100 distinct specific mutations). In some embodiments, at least 500 distinct (e.g., 500 or more) probes are used (e.g., target 500 distinct specific mutations). In some embodiments, at least 1,000 (e.g., 1,000 or more) distinct probes are used (e.g., target 1,000 distinct specific mutations). In some embodiments, at least 10,000 (e.g., 10,000 or more) distinct probes are used (e.g., target 10,000 distinct specific mutations). In some embodiments, where more than one probe is used to capture more than one distinct specific mutation, the specific mutations are in non-overlapping regions of the genome of the subject from which the DNA duplexes are obtained.

Once a probe has annealed to a single-stranded amplified DNA and the probes have been recovered along with any bound single-stranded amplified DNA to produce an enriched sample, the sample is prepared for sequencing. In some embodiments, single-stranded DNA is sequenced by duplex sequencing methods. Duplex sequencing is a type of nucleic acid sequencing that uses the information from both strands of a duplex to generate results regarding the genomic profile of a sample, or subject from which a sample was obtained. Herein, the term “duplex sequencing” is used to also embody any sequencing method that derives high accuracy by requiring a consensus of sequences from both strands of each DNA duplex, although any suitable method of nucleic acid sequencing may be used. Duplex sequencing inherently possesses the ability to provide greater accuracy regarding the sequence of the nucleic acid, as computational analysis can resolve errors by using known properties of a duplex. For example, without limitation, the understanding that nucleobases form canonical base “pairings” when part of a duplex. This property of nucleic acids has been well-known since at least the latter half of the past century, and is readily understood and appreciated by those in the art. Accordingly, employing this knowledge, it is possible to infer and determine the predicted complementary sequence from the sequencing of one strand of a duplex. This inferred complementary sequence can then be compared with the results from the sequenced second strand of nucleic acid of the duplex. When such two strands are compared, they can confirm the sequences obtained, or highlight differences, thus pinpointing possible lesions (e.g., damaged bases) or mismatches only found on one strand, or sequencing errors or areas for further investigation. These differences may result from errant base insertions, deletions, or mutations (e.g., damaged bases), Further, the results of sequenced duplexes can further be compared to reference data further providing insight into possible mutations in the sequence. Accordingly, duplex sequencing provides for a high-accuracy method of resolving the sequence of nucleic acids, in which accuracy permits greater resolution in determining the effect of differences therein (e.g., the effect of mutations in the genomic data). In some embodiments, an enriched sample is sequenced by duplex sequencing.

After sequencing, the data produced (e.g., sequencing results) may be queried by a user to identify (e.g., determine, assess, confirm) if a sequence containing a specific mutation is present. In some embodiments, a specific mutation is identified if a sequence is present in the sequencing results containing (e.g., comprising) a specific mutation. In some embodiments, a sequence containing a specific mutation may be the original top (e.g., sense, ‘+’) strand. In some embodiments, a sequence containing a specific mutation may be the original bottom (e.g., antisense, ‘-’) strand. In some embodiments, a specific mutation is identified if it appears or is contained in a sequence correlating to either the top or bottom strand. In some embodiments, a specific mutation is identified if it appears or is contained in both the top and bottom strand of the original DNA duplex. When a specific mutation appears in both strands, it is understood by the skilled artisan that the specific mutation is with respect to the base pairing, as such the sequencing will be different (as they are complementary), but will comprise the same specific mutation. Assessing the top and bottom strand to determine the pairings of sequences may be accomplished by exploiting the unique nature of the UMIs attached to each strand and unique to the duplex. After isolating the pairings, sequences may be aligned using customary tools for nucleic acid alignments (e.g., BWA, BLAST, HPC-BLAST, CS-BLAST, CUDASW++, DIAMOND, FASTA, etc.). Such methods are well-known in the art and software to perform such alignments is readily available for free use.

In some embodiments, the double-strand consensus (DSC) to single-strand consensus (SSC) is used to form a ratio. Methods for determining a consensus sequence are well known in the art, and in the context of nucleic acids is generally known to refer to the determination of an accepted sequence based on the most frequent nucleotide found at a given location in a sequence by comparing the position of a multitude of sequences subsequent to alignment. When establishing a DSC to SSC ratio, a consensus sequence is prepared for each sequence targeted by a given probe. Optimally, there will be one given consensus sequence for each set of single-stranded amplified DNA captured by a given probe, and further yet, one given consensus sequence for the complementary strand of a single-stranded amplified DNA captured by a given probe. As mentioned elsewhere in this disclosure, the strands of single-stranded amplified DNA comprise UMIs, which allow for the tracing of strands to their DNA duplex allowing for analysis of the two strands as one duplex. By exploiting this property, a consensus sequence can be established for the duplex (e.g., a double-stranded consensus sequence (DSC)). Optimally, there will only be one DSC for each set of SSCs captured by probes for a given specific mutation. Thus, an optimal DSC to SSC ratio is 0.5 (e.g., 1 DSC to 2 SSCs). However, due to imperfect capture, as well as other point mutations, sequencing errors, or errors introduced into a sequence during PCR, variations may arise in the single-stranded amplified DNA. Thus, it is improbable, if not impossible, to achieve a DSC to SSC ratio of 0.5. However, by placing a threshold on the DSC to SSC ratio, a filter is created to eliminate detection of errors that lack accuracy and/or have excess variant sequences present. In some embodiments, the DSC to SSC ratio of any of the methods of the disclosure is at least 0.1 (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, or more). In some embodiments, the DSC to SSC ratio of any of the methods of the disclosure is greater than or equal to 0.15. In some embodiments, the DSC to SSC ratio of any of the methods of the disclosure is greater than or equal to 0.2. In some embodiments, the DSC to SSC ratio of any of the methods of the disclosure is greater than or equal to 0.3.

In some embodiments, a method of the disclosure relates to methods of detecting specific mutations, wherein a specific mutation is a single nucleotide polymorphism. In some embodiments, a method of the disclosure relates to methods of detecting specific mutations, wherein a specific mutation is a structural variant.

It was observed that certain bases and/or base pairings may be more prone to error (e.g., high-noise) than other bases and/or base pairings (e.g., low-noise). By investigating the presence of low-noise mutations (e.g., those less prone to error), the likelihood that an observed specific mutation is accurate is increased. Accordingly, when establishing the specific mutations to identify using the methods of the disclosure, those comprising specific mutations at adenine (A) and/or thymine (T) sites in a reference sequence, the confidence the mutation is accurate is increased. As used herein, a site in a reference sequence refers to the location of a base pairing in a consensus sequence for a given genome (or fragment thereof). In some embodiments, methods involve tracking low-noise mutations. In other embodiments, methods involve tracking high-noise mutations. In some embodiments, low-noise mutations comprise mutations at references sites comprising A/T base pairings. In some embodiments, high-noise mutations comprise mutations at references sites comprising cytosine.

Additional steps may also be included in methods of the disclosure. For example, without limitation, a method may comprise steps to introduce controls (e.g., positive controls, controls to evaluate and/or gauge the efficiency of the method and/or the probes). In some embodiments, methods of the disclosure comprise controls. In some embodiments, a control is a positive control. As used herein, a positive control refers to creating a set of conditions in the method that is known to produce a certain result. For example, the inclusion of synthetic mutated sequences (e.g., synthetic polynucleotides) that contain a target sequence of a probe (e.g., comprise a sequence containing a specific mutation that anneals to a probe) may serve as a positive control. In some embodiments, methods of the disclosure comprise a positive control. In some embodiments, a positive control comprises a polynucleotide comprising a specific mutation in a sequence that anneals to a specific probe. In some embodiments, an internal control polynucleotide further comprises an index sequence. In some embodiments, the index sequence is variable. In some embodiments, an internal control polynucleotide is further flanked on the 5′ end by a universal forward binding primer and on the 3′ end by a universal reverse binding primer. In some embodiments, an internal control polynucleotide is further flanked on the 5′ end and the 3′ end by sequencing adapters. In some embodiments, an internal control polynucleotide is further flanked on the 5′ end by a universal forward binding primer and on the 3′ end by a universal reverse binding primer, and the binding primers are further flanked at the distal ends (e.g., 5′ and 3′ end of the construct) by sequencing adapters. By using such polynucleotides, with indexes and appropriate binding primers and sequencing adapters (cumulatively a synthetic mutant) a control can be established by including the synthetic mutant with the DNA duplexes and/or enriched sample prior to probe capture. If a probe does not capture the synthetic mutant targeted by the probe, problems may be indicated in the method and/or conditions. If the synthetic mutant is captured, but no single-stranded amplified DNA is captured, the positive control serves to validate a method and the absence of such single-stranded amplified DNA. Use of the index of the synthetic mutant allows for tracking of multiple synthetic mutants against multiple probes (e.g., for multiple target sequences comprising specific mutations). In some embodiments, a distinct synthetic mutant is used for each distinct probe and/or distinct specific mutation.

In some embodiments, internal controls comprise a fixed number, but more than one, of synthetic mutants for a single probe (e.g., single specific mutation), wherein each synthetic mutant comprises a unique index. By using more than one, but of a known number, synthetic mutant for a given specific mutation (e.g., target sequence), each with a unique index, a method can evaluate (e.g., assess, quantify) the capture efficiency of a probe. For example, the number of uniquely synthetic mutants captured can be assessed against the number of specific mutations (e.g., real mutants) captured by the probes. This property can be used for each specific mutation of a method (e.g., for multiple, more than one). In some embodiments, a set of internal controls is used for each distinct probe, wherein each set of synthetic mutants is targeted by a probe for a specific mutation, comprises a known fixed number, and comprises a unique index.

In some embodiments, the term internal is used to describe the property that these controls are placed with the DNA duplexes and/or enriched sample and are sequenced with the single-stranded amplified DNA (e.g., internal controls). The term internal controls shall be understood to include all of the aforementioned control types and variations.

In some embodiments, a specific mutation can be identified, or duplex selected, with at least 10 times (e.g., 10, 10², 10³, 10⁴, 10⁵, 10⁶) fewer sequencing reads as compared with conventional duplex sequencing methods using the methods of the disclosure. In some embodiments, a specific mutation can be identified, or duplex selected, with at least 50 times fewer sequencing reads as compared with conventional duplex sequencing methods using the methods of the disclosure. In some embodiments, a specific mutation can be identified, or duplex selected, with at least 100 times fewer sequencing reads as compared with conventional duplex sequencing methods using the methods of the disclosure. In some embodiments, a specific mutation can be identified, or duplex selected, with at least 500 times fewer sequencing reads as compared with conventional duplex sequencing methods using the methods of the disclosure. In some embodiments, a specific mutation can be identified, or duplex selected, with at least 1,000 times fewer sequencing reads as compared with conventional duplex sequencing methods using the methods of the disclosure. In some embodiments, a specific mutation can be identified, or duplex selected, with at least 10,000 times fewer sequencing reads as compared with conventional duplex sequencing methods using the methods of the disclosure. In some embodiments, a specific mutation can be identified, or duplex selected, with at least 100,000 times fewer sequencing reads as compared with conventional duplex sequencing methods using the methods of the disclosure.

### MAESTRO Probes

Probes associated with the disclosure are helpful in identifying specific mutations (and/or low-abundance mutations) in DNA duplexes and/or enriched samples, such as samples derived from subjects.

In some embodiments, the probe of any of the methods of the disclosure is 10-60 nucleotides long (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides long). In some embodiments, the probe of any of the methods of the disclosure is about 15 to about 50 nucleotides long. In some embodiments, the probe of any of the methods of the disclosure is about 20 to about 40 nucleotides long. In some embodiments, the probe of any of the methods of the disclosure is about 12 to about 32 nucleotides long. In some embodiments, the probe of any of the methods of the disclosure is about 28 to about 32 nucleotides long. In some embodiments, the probe of any of the methods of the disclosure is 30 nucleotides long.

The probes of the disclosure can be of any configuration known in the art. For example, without limitation, the probes may comprise nucleotides of deoxyribose (e.g., DNA) and/or ribose (e.g., RNA). In some embodiments, a probe comprises DNA. In some embodiments, at least one nucleotide of the probe comprises a modification (e.g., an alteration or change to at least one component of the nucleotide (e.g., nucleobase, sugar, or phosphate group)). In some embodiments, a probe contains no modified nucleotides.

In some embodiments, the probes comprise an additional moiety. A moiety may be a marker or tag. A “marker” or “tag” as used herein, refers to a molecule (e.g., nucleic acid, protein, etc.) that can be used to identify the probe in vitro and/or in vivo. Markers or tags may be any composition or molecule (e.g., nucleic acid, amino acid, peptide (e.g., glycosylated proteins, oxine, fluorescent proteins (e.g., green and/or red fluorescent protein), structures (e.g., tetracysteine loops, epitopes), any of which may be natural or synthetic (e.g., synthetic nucleic acids, amino acids, peptides, etc.)) that may be detected in vivo, in vitro, ex vivo, visually, or by exploitation of a property of the tag (e.g., fluorescence, magnetism, radioactivity, size, affinity, enzyme activity, etc.). A moiety may further be used to recover or isolate the probe, and by extension, any molecules bound thereto. In some embodiments, a moiety is a recovery moiety, wherein the moiety has a property that can be isolated and/or manipulated to separate the probe based on such property. For example, without limitation, the moiety may comprise a magnetic, chemical, physical, or affinity property that may be useful in separating the probe from extraneous material not possessing this property. Examples of such moieties are well-known in the art and any such moieties suitable may be used herein. For example, without limitation, a recovery moiety may comprise biotin. In some embodiments, an additional moiety is attached to the probe through the 5′ nucleotide. In some embodiments, a recovery moiety is attached to the probe through the 5′ nucleotide. In some embodiments, attachment is via a covalent bond.

In some embodiments, a probe comprises a nucleic acid sequence that is specific to (e.g., targets for binding) a target sequence. In some embodiments, a target sequence is representative of a specific mutation (e.g., a sequence of nucleotides equivalent to a reference sequence, but for comprising a mutation). In other words, the probe is designed to target a complementary sequence, wherein that complementary sequence comprises a specific mutation as compared to a reference sequence. In some embodiments, a specific mutation is associated or related to a disorder. Accordingly, if the probe binds this target sequence (e.g., comprising the specific mutation) it is indicative of the presence of the nucleic acid data associated with the disorder.

In some embodiments, the sequence portion of the probe that binds the specific mutation, target sequence, or SNP is located within the middle 50% of nucleotides comprising the probe, or in other words, the portion of the probe comprising the nucleotides not in the first quarter of nucleotides of the probe (e.g., the quarter comprising the 5′ end), or last quarter of nucleotides of the probe (e.g., the quarter comprising the 3′ end). In some embodiments, the sequence portion of the probe that binds the specific mutation, target sequence, or SNP is located within the middle third of nucleotides comprising the probe, or in other words, the portion of the probe comprising the nucleotides not in the first third of nucleotides of the probe (e.g., the third comprising the 5′ end), or last third of nucleotides of the probe (e.g., the third comprising the 3′ end).

In some embodiments, the nucleotide of the probe that binds the specific mutation or SNP is located within the middle 50% of nucleotides comprising the probe, or in other words, the portion of the probe comprising the nucleotides not in the first quarter of nucleotides of the probe (e.g., the quarter comprising the 5′ end) or last quarter of nucleotides of the probe (e.g., the quarter comprising the 3′ end). In some embodiments, the nucleotide of the probe that binds the specific mutation or SNP is located within the middle third of nucleotides comprising the probe, or in other words, the portion of the probe comprising the nucleotides not in the first third of nucleotides of the probe (e.g., the third comprising the 5′ end) or last third of nucleotides of the probe (e.g., the third comprising the 3′ end). In some embodiments, the nucleotide of the probe that binds the specific mutation or SNP is located within the middle 6% of nucleotides comprising the probe, or in other words, the portion of the probe comprising the nucleotides not in the first 47% of nucleotides of the probe (e.g., the 47% comprising the 5′ end) or last 47% of nucleotides of the probe (e.g., the 47% comprising the 3′ end).

In some embodiments, an allele-specific probe is evaluated and modified to increase/decrease the Gibbs free energy (AG) of the allele-specific probe annealing to its complementary sequence. By controlling and/or modifying this property of the probe, the specificity and ability for the probe to more precisely discriminate sequences and single-stranded amplified DNA can be modulated (e.g., increased, decreased). Further, by controlling this property, the stability of bound probes can also be modulated (e.g., increase, decreased). In some embodiments, the Gibbs free energy (ΔG) of an allele-specific probe annealing to its complementary sequence is at least −25 kcal/mol at Temp=50 °C, but no more than −5 kcal/mol at Temp=50 °C (e.g., −25, −24, −23, −22, −21, −20, −19, −18, −17, −16, −15, −14, −13, −12, −11, −10, −9, −8, −7, −6, −5, or increment therein). In some embodiments, the Gibbs free energy (ΔG) of an allele-specific probe annealing to its complementary sequence is at least −23 kcal/mol at Temp=50 °C, but no more than −7 kcal/mol at Temp=50 °C. In some embodiments, the Gibbs free energy (ΔG) of an allele-specific probe annealing to its complementary sequence is at least-21 kcal/mol at Temp=50 °C, but no more than −9 kcal/mol at Temp=50 °C. In some embodiments, the Gibbs free energy (ΔG) of an allele-specific probe annealing to its complementary sequence is at least −20 kcal/mol at Temp=50 °C, but no more than −12 kcal/mol at Temp=50 °C. In some embodiments, the Gibbs free energy (ΔG) of an allele-specific probe annealing to its complementary sequence is at least −19 kcal/mol at Temp=50 °C, but no more than −13 kcal/mol at Temp=50 °C. In some embodiments, the Gibbs free energy (ΔG) of an allele-specific probe annealing to its complementary sequence is at least −18 kcal/mol at Temp=50 °C, but no more than-14 kcal/mol at Temp=50 °C. In some embodiments, the Gibbs free energy (ΔG) of an allele-specific probe annealing to its complementary sequence is at least −17 kcal/mol at Temp=50 °C, but no more than −15 kcal/mol at Temp=50 °C. In some embodiments, the Gibbs free energy (ΔG) is modified by adjusting the length of the sequence of the probe that will bind a target sequence (e.g., comprising a specific mutation). In some embodiments, length is increased. In some embodiments, length is decreased. In some embodiments, the length is adjusted iteratively until the Gibbs free energy (ΔG) is within the ranges preferred. In some embodiments, the length is adjusted iteratively until the Gibbs free energy (ΔG) is within the ranges as described herein.

A further evaluation and design consideration given to constructing a probe according to the present disclosure comprises evaluating the likely ability of the probe to bind other portions of a nucleic acid (e.g., other areas, portions, fragments, of a genome). Accordingly, once a probe sequence is developed, it may be evaluated to see if it is homologous with any other areas of a genome of a subject from which the DNA duplexes and/or enriched sample were taken. There are a multitude of well-known methods, tools, and software programs publicly, and freely available to perform such searches (e.g., BLAST, etc.). In some embodiments, a target sequence of the allele-specific probe is homologous with less than 20 sequences of a reference genome of the subject. In some embodiments, a target sequence of the allele-specific probe is homologous with less than 15 sequences of a reference genome of the subject. In some embodiments, a target sequence of the allele-specific probe is homologous with less than 10 sequences of a reference genome of the subject. In some embodiments, a target sequence of the allele-specific probe is homologous with less than 5 sequences of a reference genome of the subject. In some embodiments, a target sequence of the allele-specific probe is 100% homologous with less than 20 sequences of a reference genome of the subject. In some embodiments, a target sequence of the allele-specific probe is 100% homologous with less than 15 sequences of a reference genome of the subject. In some embodiments, a target sequence of the allele-specific probe is 100% homologous with less than 10 sequences of a reference genome of the subject. In some embodiments, a target sequence of the allele-specific probe is 100% homologous with less than 5 sequences of a reference genome of the subject. If there are an excess number of sites that are homologous with the target sequence of the probe (e.g., the sequence it will bind comprising a specific mutation), a probe may be modified (e.g., altered). For example, without limitation, the sequence targeted may be frameshifted in one direction or the other relative to the position of the nucleotide(s) of the specific mutation. This modification may be performed in either direction. Further, this modification may include altering the length of the probe as well (while keeping the Gibbs free energy in an appropriate range), or the length of the probe may remain constant during this shift. In some embodiments, a sequence targeted by an allele-specific probe is moved 5 nucleotides, or less (e.g., 1, 2, 3, 4, or 5) in the 5′ direction. In some embodiments, a sequence targeted by an allele-specific probe is moved 10 nucleotides, or less (e.g., 1, 2, 3, 4, 5, 6, 7, 8, 9, or 10) in the 5′ direction. In some embodiments, a sequence targeted by an allele-specific probe is moved 5 nucleotides, or less (e.g., 1, 2, 3, 4, or 5) in the 3′ direction. In some embodiments, a sequence targeted by an allele-specific probe is moved 10 nucleotides, or less (e.g., 1, 2, 3, 4, 5, 6, 7, 8, 9, or 10) in the 3′ direction.

In some embodiments, a probe is designed and/or selected for use according to one or more methods of the present disclosure, due at least in part to its annealing temperature. For example, without limitation, in some embodiments, an allele-specific probe has an annealing temperature of at least 44 degrees Celsius (°C), but no more than 56 °C. In some embodiments, an allele-specific probe has an annealing temperature of at least 45 degrees Celsius (°C), but no more than 55 °C. In some embodiments, an allele-specific probe has an annealing temperature of at least 47 degrees Celsius (°C), but no more than 54 °C. In some embodiments, an allele-specific probe has an annealing temperature of at least 48 degrees Celsius (°C), but no more than 52 °C. In some embodiments, an allele-specific probe has an annealing temperature of at least 49 degrees Celsius (°C), but no more than 51 °C. In some embodiments, an allele-specific probe has an annealing temperature of at least 50 degrees Celsius (° C). In still other embodiments, the allele-specific probe has an annealing temperature of at least 40 °C, of at least 41 °C, of at least 42 °C, of at least 43 °C, of at least 44 °C, of at least 45 °C, of at least 46 °C, of at least 47 °C, of at least 48 °C, of at least 49 °C, of at least 50 °C, of at least 51 °C, of at least 52 °C, of at least 53 °C, of at least 54 °C, of at least 55 °C, of at least 56 °C, of at least 57 °C, of at least 58 °C, of at least 59 °C, of at least 60 °C, of at least 61 °C, of at least 62 °C, of at least 63 °C, of at least 64 °C, of at least 65 °C, of at least 66 °C, of at least 67 °C, of at least 68 °C, of at least 69 °C, of at least 70 °C, of at least 71 °C, of at least 72 °C, of at least 73 °C, or of at least 74 °C but not more than 75 °C, or but not more than 50 °C, but not more than 51 °C, but not more than 52 °C, but not more than 53 °C, but not more than 54 °C, but not more than 55 °C, but not more than 56 °C, but not more than 57 °C, but not more than 58 °C, but not more than 59 °C, but not more than 60 °C, but not more than 61 °C, but not more than 62 °C, but not more than 63 °C, but not more than 64 °C, but not more than 65 °C, but not more than 66 °C, but not more than 67 °C, but not more than 68 °C, but not more than 69 °C, or not more than 70 °C.

In some embodiments, a recovery moiety is attached to the 5′ end of an allele-specific probe. In some embodiments, a minor groove binder (MGB) is attached to the 3′ end of an allele-specific probe. In some embodiments, a recovery moiety is biotin. However, it should be noted that any suitable appropriate tag or moiety providing a means or property by which the probe (and any single-stranded amplified DNA bound thereto) may be separated and/or recovered may be used. Appropriate such tags and/or moieties are well-known in the art and will be readily discernable by the skilled artisan. In some embodiments, an allele-specific probe comprises biotin. In some embodiments, biotin is recovered (e.g., captured) by exploiting its ability to preferentially bind avidin. In some embodiments, biotin is recovered (e.g., captured) by exploiting its ability to preferentially bind streptavidin. In some embodiments, biotin is recovered (e.g., captured) by exploiting its ability to preferentially bind neutravidin.

In some embodiments, the disclosure relates to an allele-specific probe, further comprising a minor groove binder (MGB). MGBs are molecules, typically crescent-shaped molecules, which selectively bind minor grooves of nucleic acids. MGBs typically bind with specific sequences and may bind non-covalently by a combination of directed hydrogen bonding to base pair edges. The MGB ODNs (+MGB) are shown to have a greater free energy difference (ΔΔG) in the MGB region as compared to the ODN absent the MGB (−MGB). In certain embodiments, the probes may be modified by any known means to increase the ΔΔG between match and mismatch, such as by using a locked nucleic acid; a peptide nucleic acid; a Super G, C, T, A (e.g., available or obtainable commercially); XNA nucleotides; and so forth.

Additionally, the MGBs are still effective at discriminating and binding target sequences at dilutions that are increasingly small (e.g., 1 copy). Finally, MGBs are shown to increase the melting temperature (Tₘ) of bound ODN in various configurations, Mismatches±, MGB±, wherein ODNs with no mismatches and MGBs show an elevated Tₘ. Thus, the addition of MGBs to the probes of the disclosure will improve affinity and specificity, further improving the resolution and sensitivity of the methods herein. In some embodiments, an allele-specific probe comprises an MGB.

In some embodiments, the disclosure includes methods of making allele-specific probes, the method comprising: for each target sequence (e.g., sequence comprising a specific mutation), a 30-nucleotide probe is created with the altered base (e.g., nucleotide targeting the specific mutation, e.g., the nucleotide complementary to the specific mutation) at its center. The probe may be designed against the plus strand or the minus strand depending on the base change. The length is adjusted until the estimated delta G of the probe sequence is within an acceptable range (yielding probe candidates between 20 and 40 nucleotides in length). This same strategy is used while shifting the probe's center up to 5 bp in either direction to create multiple candidates for each target. A BLAST search is performed and the candidate with the highest specificity for the target is selected. A given target may be removed from the design if its probe characteristics (delta G, length, % GC, melting temperature, number of BLAST hits) do not meet pre-specified criteria.

In some aspects, the disclosure includes methods of making an allele-specific probe, the method comprising: (a) identifying a specific mutation in a nucleic acid sequence of a genome; (b) generating a complementary nucleic acid (CNA) including a complementary base to the specific mutation; and (c) attaching a recovery moiety to the 5′ nucleotide of the allele-specific probe; wherein the complementary base is in the middle 50% of nucleotides of the CNA; wherein the CNA comprises at least 12, but no more than 60 nucleotides; wherein the Gibbs free energy of the CNA and the nucleic acid comprising the specific mutation is at least −20, but no more than-12; wherein the annealing temperature of the allele-specific probe is at least 48 degrees Celsius (° C), but no more than 52 °C; and wherein the CNA is 100% homologous with less than 10 sequences within the genome.

### Treatment of Cancer

Aspects of the disclosure relate to treatment of cancer patients. In some embodiments, Dynamic Minimal Residual Disease Detection is used to monitor treatment efficacy in a patient who has cancer. Many types of cancer are able to evade therapy by undergoing molecular changes, such as mutations or pathway alterations. Dynamic Minimal Residual Disease Detection can be used to detect, with high sensitivity and high specificity, whether cancer cells in a patient are responding to cancer therapy. In some embodiments, Dynamic Minimal Residual Disease Detection is used to determine whether a patient who is suspected of having cancer actually has cancer. Dynamic Minimal Residual Disease Detection provides for early detection of a small number of cancer cells that otherwise would not be detected until a tumor forms. In some embodiments, Dynamic Minimal Residual Disease Detection is used to determine whether a patient who is at risk of having cancer actually has cancer. An individual who may have genetic or other risk factors for cancer may develop low levels of cancer cells prior to development of a tumor. Dynamic Minimal Residual Disease Detection provides for detection of a small number of cancer cells in a patient, earlier than other diagnostic procedures. In some embodiments, Dynamic Minimal Residual Disease Detection is used to monitor a patient who had cancer for tumor recurrence. Many cancer therapies do not completely abolish cancer in a subject and a small population of cancer cells can remain after a tumor has been removed from a patient. This small population of cancer cells can rapidly divide and form a new tumor after a patient's treatment plan has ended. Dynamic Minimal Residual Disease Detection provides for monitoring for tumor recurrence in a patient. Dynamic Minimal Residual Disease Detection can be used in many aspects of cancer patient care. In some embodiments, the results of Dynamic Minimal Residual Disease Detection inform a physician whether to initiate cancer treatment, intensify cancer treatment, de-escalate cancer treatment, change cancer treatment, and/or halt or end cancer treatment. Dynamic Minimal Residual Disease Detection can be used to initiate a patient's cancer treatment. In some embodiments, if a patient who is not being treated for cancer has a sample determined to be MRD-positive, then the patient can subsequently undergo treatment for cancer, thus providing early diagnosis. In some embodiments, if a patient who is being treated for cancer has a sample determined to have an elevated MRD value, then the treatment can be subsequently intensified or changed. In some embodiments, if a patient who is being treated for cancer has a sample determined to be MRD-negative, then the treatment can be subsequently de-escalated or halted. In some embodiments, if a patient who is being treated for cancer by administration of one or more chemotherapeutics has a sample determined to have an elevated MRD value, then the patient can be subsequently treated with one or more different chemotherapeutics. In some embodiments, one or more chemotherapeutics comprise dabrafenib, trametinib, ipilimumab, and/or nivolumab.

In some embodiments, a patient is treated according to an initial treatment plan and then when a sample from the patient is found to be positive for MRD, the initial treatment plan is altered to create an updated treatment plan. In some embodiments, the initial treatment plan comprises administering a first drug at a first dosage to the patient; altering the initial treatment plan comprises changing (increasing or decreasing) the first dosage to a second dosage; and an updated treatment plan comprises administering the first drug at the second dosage to the patient. In some embodiments, the initial treatment plan comprises administering a first drug to the patient; altering the initial treatment plan comprises changing the first drug to a second drug or providing the first drug in combination with the second drug; and an updated treatment plan comprises administering the first drug, the second drug, or a combination thereof to the patient.

In some embodiments, a patient is treated according to an initial treatment plan and if a sample from the patient is found to be negative for MRD, the initial treatment plan is altered to create an updated treatment plan. In some embodiments, the initial treatment plan comprises administering a first drug at a first dosage to the patient; altering the initial treatment plan comprises increasing or decreasing the first dosage to a second dosage; and the updated treatment plan comprises administering the first drug at the second dosage to the patient. In some embodiments, the initial treatment plan comprises administering a first drug to the patient; altering the initial treatment plan comprises eliminating the first drug from the initial treatment plan; and an updated treatment plan comprises treating the patient without administering the first drug to the patient or stopping treatment of the patient.

Dynamic Minimal Residual Disease Detection can be used to determine whether a patient sample is MRD-positive or MRD-negative regardless of the type of cancer a patient has. In some embodiments, a cancer is blood or lymphoid cancer, a bone or soft tissue cancer, a brain or central nervous system cancer, breast cancer, a childhood cancer, a digestive system cancer, an eye cancer, a head or neck cancer, lung cancer, a pelvic area cancer, a skin cancer, or a urinary cancer. In some embodiments, a cancer is a glioblastoma.

### Subjects

The term “subject,” as used herein, refers to any organism in need of treatment or diagnosis using the subject matter herein. For example, without limitation, subjects may include mammals and non-mammals. In some embodiments, a subject is mammalian. In some embodiments, a subject is non-mammalian. As used herein, a “mammal,” refers to any animal constituting the class Mammalia (e.g., a human, mouse, rat, cat, dog, sheep, rabbit, horse, cow, goat, pig, guinea pig, hamster, chicken, turkey, or a non-human primate (e.g., Marmoset, Macaque)). In some embodiments, a mammal is a human. In some embodiments, a subject is under the care and/or direction of a medical professional (e.g., a patient). In some embodiments, a subject is a patient. In some embodiments, a subject has, is at risk of having, has had previously, or is suspected of having cancer. In some embodiments, a subject is a subject that has a tumor, a subject that had a tumor in the past, a subject at risk of having a tumor, or a subject that is suspected of having a tumor. In some embodiments, a tumor is cancerous.

### Kits

In an aspect, the disclosure relates to kits for performing one or more of the methods of the disclosure (e.g., identification of specific mutations and/or low-abundance mutations) in a sample of DNA duplexes and/or enriched sample and/or determining whether a patient sample is MRD positive.

In some embodiments, a kit comprises materials and/or reagents to carry out one or more of the methods of the disclosure. For example, without limitation, the kit may comprise the components and/or reagents to perform the entire method, and/or any portion thereof. In some embodiments, materials and devices are provided in the kits that provide for the acquisition and/or procurement of a sample of DNA duplexes. In some embodiments, a kit comprises devices and/or housings (e.g., containers) to hold any of the liquid stages or materials of one or more methods of the disclosure.

In some embodiments, a kit comprises any of the probes as described herein useful for one or more of the methods of the disclosure.

In some embodiments, a kit comprises materials and/or reagents to carry out the method of making an allele-specific probe according to the instant disclosure. In some embodiments, a kit comprises a probe as produced by the methods of the disclosure.

In some embodiments, a kit comprises materials, devices, and/or reagents to carry out a liquid biopsy to detect one or more mutations.

Instructions for performing one or more of the methods of the disclosure may also be included in the kits described herein.

The kit may contain packaging or a container with components as described herein.

Other suitable components to include in such kits will be readily apparent to one of skill in the art, taking into consideration the desired application and use of one or more of the methods of the disclosure.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Unless otherwise defined herein, scientific and technical terms used in connection with the present disclosure shall have the meanings that are commonly understood by those of ordinary skill in the art (e.g., the skilled artisan). The meaning and scope of the terms are clear; however, in the event of any latent ambiguity, definitions provided herein take precedence over any dictionary or extrinsic definition. Further, unless otherwise indicated by context, singular terms shall include pluralities and plural terms shall include the singular. In this disclosure, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present disclosure unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of subjects.

The terms “approximately” or “about,” as may be used interchangeably herein, and as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction of (i.e., percentage greater than or percentage less than) the stated reference value unless otherwise stated or otherwise evident from the context (for example, when such number would exceed 100% of a possible value).

### EXAMPLES

### Example 1. MAESTRO-Pool Enables Highly Parallel and Specific Mutation-Enrichment Sequencing for Minimal Residual Disease Detection in Cohort Studies

Tracing patient-specific tumor mutations in cell-free DNA (cfDNA) for minimal residual disease (MRD) detection is promising but challenging. Assaying more mutations and cfDNA stands to improve MRD detection but requires highly accurate, efficient sequencing methods and proper calibration to prevent false detection with bespoke tests.

MAESTRO (Minor Allele Enriched Sequencing Through Recognition Oligonucleotides) uses mutation-specific oligonucleotide probes to enrich cfDNA libraries for tumor mutations and enable their accurate detection with minimal sequencing. A new approach, MAESTRO-Pool, which entails pooling MAESTRO probes for all patients and applying these to all samples from all patients, was used to screen for 22,333 tumor mutations from 9 melanoma patients in 98 plasma samples. This enabled quantification of MRD detection in patient-matched samples and false detection in unmatched samples from other patients. To detect MRD, a new dynamic MRD caller was used that computes a probability for MRD detection based on the number of mutations and cfDNA molecules sequenced, thereby calibrating for variations in each bespoke test.

MAESTRO-Pool enabled sensitive detection of MRD down to 0.78 parts per million (ppm), reflecting a 10- to 100-fold improvement over existing tests. Of the 8 MRD positive samples with ultra-low tumor fractions<10 ppm, 7 were either in upward-trend preceding recurrence or downward-trend aligning with response. Of 784 patient-unmatched tests, only one was found as MRD positive (tumor fraction=2.7 ppm), suggesting high specificity.

MAESTRO-Pool enables massively parallel, tumor-informed MRD testing with concurrent benchmarking of bespoke MRD tests. Meanwhile, the new MRD caller enables more mutations and cfDNA molecules to be tested without compromising specificity. These improve the ability for detecting traces of MRD from blood.

### Introduction

Using circulating tumor DNA (ctDNA) to detect minimal residual disease (MRD) from blood stands to inform better care for cancer patients. It could enable early intensification or switching of therapy long before clinical relapse, or de-escalation of therapy when no longer needed, thereby sparing the side effects of therapy. Multiple studies have shown that ctDNA detection is highly predictive for tumor recurrence. However, the sensitivity of detecting MRD at key clinical time points such as within a few months following surgery is low, often below 25%, especially in cancer types considered to be “low ctDNA shedders.” Given this, there is an unmet need to improve sensitivity to the maximum possible levels. Being able to detect MRD in more patients and with longer lead time to recurrence could enable ctDNA testing to inform therapy escalation and de-escalation for cancer patients.

Higher sensitivity for detecting MRD is attainable by tracking more tumor mutations per patient or sampling more cell-free DNA (cfDNA) molecules from blood. Given the genetic diversity of most patients' tumors, tracking more tumor mutations involves “tumor-informed” assays: sequencing a patient's tumor, identifying a unique tumor mutation fingerprint, and assaying for this fingerprint in each patient's own plasma DNA. Yet, this becomes challenging due to the massive excess of normal cfDNA in blood and the extreme amounts of sequencing required to accurately resolve low-abundance mutations. Further, as bespoke assays are developed to detect increasingly lower tumor fractions in cfDNA, it becomes important to mitigate the potential for false detection. This has been largely underexplored as bespoke assays are generally not benchmarked in many samples on an individualized basis. It was reasoned that plasma samples from other patients could be used as controls for one another, coupled with improved methods for MRD detection, to characterize and safeguard against false detection.

Here, MAESTRO-Pool is introduced, a modification of the recently developed MAESTRO (Minor Allele Enriched Sequencing Through Recognition Oligonucleotides) method for mutation enrichment sequencing of rare mutations, which has been used to improve MRD detection from liquid biopsies. With MAESTRO-Pool, MAESTRO probes from a cohort of patients are synthesized, pooled, and subjected to all samples from all patients, thereby enabling the detection of MRD in patient-matched samples and the quantification of false detection in patient-unmatched samples. A new “dynamic” MRD caller that provides a probabilistic score for MRD detection is also introduced, allowing more mutations and cfDNA molecules to be sequenced while limiting false detection.

### Materials and Methods

### Patients and Samples

All patients provided written, informed consent to allow the collection of blood and tumor tissue and analysis of clinical and genetic data for research purposes. Patients with high-risk melanoma were prospectively identified for enrollment into an Institutional Review Board-approved tissue analysis and banking cohort of which 9 were selected for analysis. DNA was extracted and made into sequencing libraries as previously described.

### Designing Tumor Fingerprints

For MAESTRO tumor fingerprints, patient-specific fingerprint design followed the method previously described. Tumor DNA was extracted from fresh frozen tissue and submitted for 30× whole-genome sequencing (WGS) via the Illumina® NovaSeq™ S4 while paired normal DNA was extracted from the buffy coat and submitted for 15×WGS. Using the paired tumor-normal WGS data, somatic variants were called with the GATK4 Best Practices Mutect 2 workflow and used as input into the MAESTRO Probe Design Tool to generate a candidate list for each patient's tumor fingerprint. These lists were further filtered by removing targeted loci overlapping low complexity regions or common germline variants to curtail against false positives due to poor alignment or contamination, respectively. Then, to create the MAESTRO-Pool assay, all the individual fingerprints were combined and subjected to the following filters: (1) if two or more patients have probes targeting the same locus but different alleles or (2) if two or more patients have probes targeting loci within 200 bp of each other, keep the probe from the patient with the smaller fingerprint. However, probes targeting the same locus and allele in multiple patients were allowed to be included. The resulting MAESTRO-Pool probe set was ordered as an o-pool product from IDT.

Additionally, the optimized Parsons et al. method was used to design personalized MRD Tracker assays that do not utilize MAESTRO enrichment. Because of the substantial sequencing requirements compared to MAESTRO, MRD Tracker fingerprints were capped at 1000 probes per patient, which were prioritized via WGS variant allele frequency. Then, each patient's probe set was ordered as an xGen™ Custom Hyb Panel product from IDT.

### Sample Processing

cfDNA was extracted from 4-7 ml plasma using the QIAsymphony® Circulating DNA kit and quantified using the Quant-iT™ PicoGreen™ assay on a Hamilton® STAR-line liquid handling system. cfDNA and gDNA libraries were constructed using the Kapa™ Hyper Prep kit with custom dual index duplex UMI adapters (IDT), as previously described. The prepared libraries were then quantified using the Quant-iT™ PicoGreen™ assay on a Hamilton® STAR-line liquid handling system.

### Hybrid Capture and Sequencing

MAESTRO-Pool hybrid capture was performed by following the previously published method. In brief, hybrid capture was performed using xGen™ Hybridization and Wash Kit with xGen™ Universal Blockers (IDT). Each hybrid capture contained a maximum of 12 samples, with a library mass equivalent to 50 times DNA mass into library construction for each sample and used 4 μmol of the MAESTRO-Pool panel. The hybridization program began at 95 °C for 30 seconds, followed by a stepwise decrease in temperature from 65 °C to 50 °C, dropping 1 °C every 48 minutes. Finally, the plate was held at 50 °C for at least four hours. Heated wash steps were performed at 50 °C. After the first round of hybrid capture, 16 cycles of PCR were applied. The product was subject to a second round of hybrid capture using 2 μmol of the MAESTRO-Pool panel. This was followed by another 16 cycles of PCR. Final captured product was quantified and pooled for sequencing on an Illumina® NovaSeq™ S4 (151 bp paired end reads) with a target raw depth of 50 million reads per sample.

MRD Tracker hybrid capture was performed by following the previously published method with some notable differences from MAESTRO-Pool above, including: (1) A library mass equivalent to 25 times DNA mass into library construction was used for hybrid capture; (2) The hybridization program began at 95 °C for 30 seconds, followed by staying at 65 °C for at least four hours; (3) Heated wash steps were performed at 65 °C. Final captured product was sequenced on an Illumina® NovaSeq™ S4 with a target raw depth of 40,000× per site per 20 ng DNA mass into library construction.

### Data Processing and Filtering

Sequencing and consensus calling followed the same protocol previously described. The resulting consensus data for each MAESTRO-Pool sample was then used as input into Miredas, a suite of custom MRD calling scripts, which applied additional fragment-level filtering and quantified the number of consensus molecules per site. After generating these site-specific counts for every sample, each patient's validated tumor fingerprint was determined to ensure that robust, tumor-specific mutations were being tracked. Sites that did not meet all the following criteria were excluded from all downstream analyses: (1) 0 ALT duplexes in the matched normal gDNA, (2) >0 ALT duplexes in the matched tumor gDNA, and (3) >0.15 for the ratio of ALT duplexes to ALT single strand consensus molecules (i.e., DSC/SSC ratio) in the matched tumor gDNA. With each patient's validated tumor fingerprint defined, matched and unmatched samples were distinguished for the final site-level filtering step. For matched samples, the following criteria were used for each site to be considered for MRD detection: (1) passed tumor validation and (2) >0.15 for the DSC/SSC ratio if >0 ALT duplexes. However, a different set of filters was applied for unmatched samples since these samples were used for estimating false detection rather than true tumor signal. Therefore, the following criteria were used for each site to ensure detection was not impacted by any germline or somatic factors: (1) target site passed tumor validation for fingerprint's patient, (2) target site not in sample's matched fingerprint, (3) 0 ALT duplexes in sample's matched normal gDNA, (4) 0 ALT duplexes in sample's matched tumor gDNA, and (5) >0.15 for the DSC/SSC ratio if >0 ALT duplexes in cfDNA sample.

MRD Tracker samples underwent similar processing as the MAESTRO-Pool matched samples. They were processed using the same consensus calling workflow and Miredas steps. They also went through tumor validation filtering but without the DSC/SSC ratio filter, which is specific to MAESTRO data.

Lastly, MAESTRO and MRD Tracker samples were flagged for semi-automated review if there were between 2-10 mutations detected. In these cases, each detected mutation was reviewed to determine if the mutation was likely false (e.g., alignment artifact), in which case it was discarded from the validated tumor fingerprint.

### Tumor Fraction Estimation

Tumor fraction estimation for MAESTRO-Pool and MRD Tracker samples relied on methods described previously. However, only sites that passed all filtering were considered for tumor fraction estimation. MAESTRO samples assumed a consistent duplex depth across all pass filter sites, which were generated from the 10 least enriched sites, as described in previous work.

### Dynamic MRD Calling

The goal for dynamic MRD calling was to devise a method that would weigh sample-specific attributes, such as observed tumor fraction, validated fingerprint size, and cfDNA mass, and quantify the probability of the observed data being the result of true tumor signal rather than false, spontaneous errors. Additionally, the output is intended to be easily interpretable. As a result, the following model was designed to be used in addition to the previous criterion of ≥2 mutated duplexes across ≥2 sites:

$$P ( {M}^{+} \vert D ) = \frac{P ( D \vert {M}^{+} ) P ( {M}^{+} )}{{\sum }_{i ∈ \{ + , - \}} P ( D \vert {M}^{i} ) P ( {M}^{i} )} \qquad ( 1 )$$

$$P ( D \vert {M}^{+} ) = β ( \# ALT duplexes , \# total duplexes , t ) \qquad ( 2 )$$

$$where t = max ( \frac{\# ALT}{\# total} , error rate )$$

$$P ( D \vert {M}^{-} ) = β ( \# ALT duplexes , \# total duplexes , error rate ) \qquad ( 3 )$$

$$\underline{Assumptions}$$

$$P ( {M}^{+} ) = P ( {M}^{-} ) = 0.5$$

$$error rate = 1 \times {10}^{- 7}$$

The output of the model, seen in Equation 1, was the probability of the sample being MRD positive given the observed data, which lent itself to being easily calculated via Bayes' Theorem, interpretable and tunable. The sample-specific inputs to the model included the total mutated duplexes and the total assumed duplexes across sites passing all filters. The total assumed duplexes were estimated using the 10 least enriched sites as described in the Tumor Fraction Estimation section. Notably, this variable indirectly integrated attributes like fingerprint size and cfDNA mass, enabling the model to adapt its confidence easily to the particular sample. To estimate the likelihoods of observing the data given that the sample was MRD positive or negative, it was assumed that sampling mutated duplexes from cfDNA could be modeled as a binomial distribution. With Equation 2, the likelihood of the mutated duplexes being exclusively tumor derived was calculated. For this, a detection floor equal to the background mutation frequency was established, ensuring that the model did not weight detection rates below the background mutation frequency as tumor derived. Similarly, the likelihood of the mutated duplexes being spontaneous errors was calculated (Equation 3). A default error rate was chosen to be 1×10⁻⁷, which was derived from error rates in previous MAESTRO datasets (ranging from 1×10⁻⁸-1×10⁻⁷). Lastly, uninformative priors were assumed for samples being MRD positive or negative, reasoning that plasma samples taken in the MRD monitoring setting have a fair chance of being either positive or negative.

### Limit of Detection Estimation

The limit of detection represented the tumor fraction that was 90% powered to be detected given the number of duplexes observed per site. However, previous estimations assumed the fixed MRD calling strategy of detecting ≥2 mutated duplexes across ≥2 sites. This framework was adjusted to find the tumor fraction at 90% power when using dynamic MRD calling. This consisted of first finding the minimum number of mutated duplexes for a particular sample to have P≥0.95. Then, the same logic as previously described was used to calculate which tumor fraction had 90% power to detect at least the minimum number of mutated duplexes across ≥2 sites. In conjunction with other analyses, the duplex depth for MAESTRO samples was estimated as described in the Tumor Fraction Estimation section.

### Example Environment

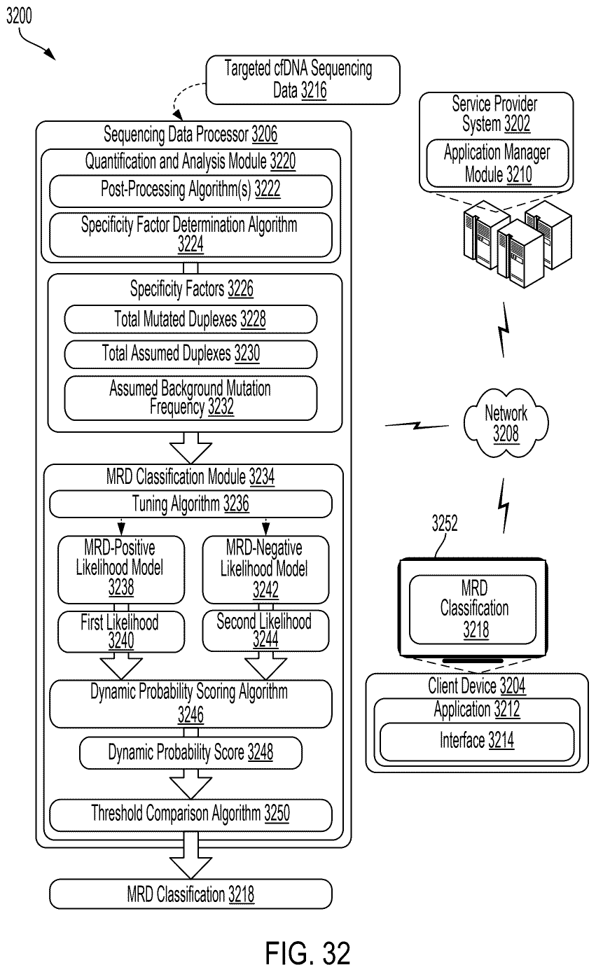

FIG. 32 is an illustration of an environment 3200 in an example implementation that is operable to employ dynamic MRD classification as described herein. The illustrated environment 3200 includes a service provider system 3202, a client device 3204, and a sequencing data processor 3206 that are communicatively coupled, one to another, via a network 3208. Although the sequencing data processor 3206 is illustrated as separate from the service provider system 3202 and the client device 3204, this functionality may be incorporated as part of the service provider system 3202 and/or the client device 3204, further divided among other entities, and so forth. By way of example, an entirety of or portions of the functionality of the sequencing data processor 3206 may be incorporated as part of the service provider system 3202 and/or the client device 3204. Additionally, or alternatively, an entirety of or portions of the client device 3204 may be incorporated as part of the service provider system 3202.

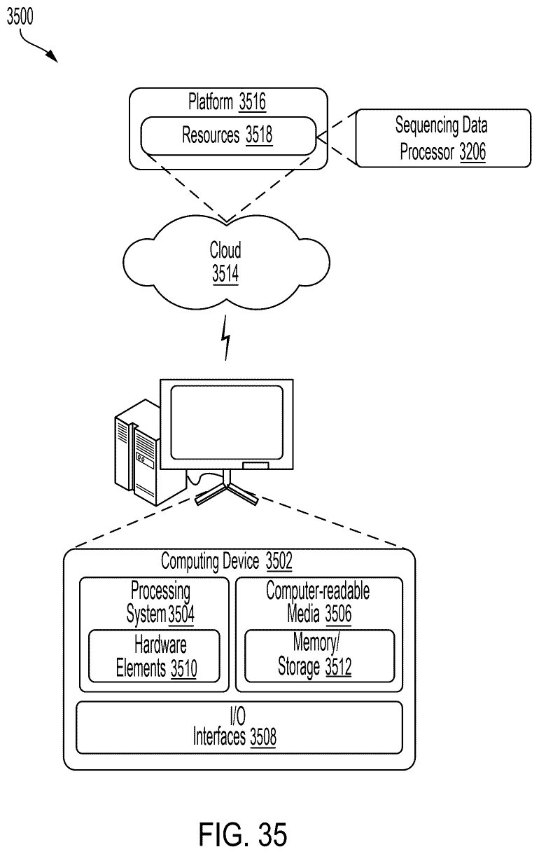

Computing devices that are usable to implement the service provider system 3202, the client device 3204, and the sequencing data processor 3206 may be configured in a variety of ways. A computing device, for instance, may be configured as a desktop computer, a laptop computer, a mobile device (e.g., assuming a handheld configuration such as a tablet or mobile phone), and so forth. Thus, the computing device may range from full resource devices with substantial memory and processor resources (e.g., personal computers, game consoles) to a low-resource device with limited memory and/or processing resources (e.g., mobile devices). Additionally, a computing device may be representative of a plurality of different devices, such as multiple servers utilized to perform operations “over the cloud,” as further described in relation to FIG. 35.

The service provider system 3202 is illustrated as including an application manager module 3210 that is representative of functionality to provide access to the sequencing data processor 3206 to a user of the client device 3204 via the network 3208. The application manager module 3210, for instance, may expose content or functionality of the sequencing data processor 3206 that is accessible via the network 3208 by an application 3212 of the client device 3204. The application 3212 may be configured as a network-enabled application, a browser, a native application, and so on, that exchanges data with the service provider system 3202 via the network 3208. The data can be employed by the application 3212 to enable the user of the client device 3204 to communicate with the service provider system 3202, such as to receive application updates and features when the service provider system 3202 provides functionality to manage the application 3212.

In the context of the described techniques, the application 3212 includes functionality to analyze data generated by at least one sequencing event. In the illustrated example, the application 3212 includes an interface 3214 that is implemented at least partially in hardware of the client device 3204 for facilitating communication between the client device 3204 and the sequencing data processor 3206. By way of example, the interface 3214 includes functionality to receive inputs to the sequencing data processor 3206 from the client device 3204 (e.g., from a user of the client device 3204) and output information, data, and so forth from the sequencing data processor 3206 to the client device 3204, as will be further elaborated herein.

The sequencing event includes determining an order of nucleotides (e.g., adenine, thymine or uracil, cytosine, and guanine) in one or more samples of nucleic acids, such as derived from one or more biological samples. The order of nucleotides is referred to herein as a “sequence.” The nucleotides are also referred to as “bases.” The sequencing event will be described herein with respect to deoxyribonucleic acid (DNA) sequencing, and particularly with respect to cell-free DNA (cfDNA), such as generated by using the MAESTRO or MAESTRO-Pool techniques described herein. See, for example, the Designing Tumor Fingerprints, Sample Processing, and Hybrid Capture and Sequencing sections described above. Such techniques produce targeted cfDNA sequencing data 3216 that is analyzed by the sequencing data processor 3206 to determine an MRD status of the corresponding sample. For instance, the corresponding sample is classified as MRD positive (e.g., circulating tumor DNA is present) or MRD negative (e.g., circulating tumor DNA is not present) by the sequencing data processor 3206. The MRD status may be output as an MRD classification 3218. In at least one implementation, the targeted cfDNA sequencing data 3216 comprise a text-based file format, such as FASTQ files that store both nucleotide sequence information and quality scores for the bases in a sequencing read. In variations, the targeted cfDNA sequencing data 3216 comprise another type of file format.

In at least one variation, the corresponding sample is classified as being positive for circulating tumor DNA or negative for circulating tumor DNA rather than the MRD classification described above. As such, it is to be appreciated that the described techniques are applicable to detecting circulating tumor DNA outside of the context of MRD.

In at least one implementation, the sequencing data processor 3206 receives the targeted cfDNA sequencing data 3216 and performs data processing and filtering via a quantification and analysis module 3220. The quantification and analysis module 3220 is representative of functionality for identifying single nucleotide variants (SNVs) in the targeted cfDNA sequencing data 3216 to use for determining the MRD classification 3218. Accordingly, in at least one implementation, the quantification and analysis module 3220 includes one or more post-processing algorithms 3222 to identify the sites (e.g., the SNVs) for MRD detection (see, for example, the Data Processing and Filtering section described above). By way of example, the targeted cfDNA sequencing data 3216 may be processed by the one or more post-processing algorithms 3222 to identify reads that match the targeted variants of the MAESTRO probes while background noise and sequencing errors are filtered out to enhance the detection of true variants.

The quantification and analysis module 3220 may further include a specificity factor determination algorithm 3224. The specificity factor determination algorithm 3224 may quantify specificity factors 3226 that are used in determining the MRD classification 3218, as further described herein. In the example of the environment 3200, the specificity factors 3226 include total mutated duplexes 3228 and total assumed duplexes 3230. The total mutated duplexes 3228 refers to the count of DNA duplexes in the targeted cfDNA sequencing data 3216 of a sample that contain the specific variants of interest across sites passing all filters. The total mutated duplexes 3228 are also referred to as alternative (“ALT”) duplexes herein. The total assumed duplexes 3230 refers to the total count of DNA duplexes, including both mutant and wild-type (non-mutant) duplexes, that are assayed for mutations in the sample. In at least one implementation, the total assumed duplexes 3230 is estimated using a pre-determined number (e.g., 10) of the least enriched sites, as described in the Tumor Fraction Estimation section. For example, the total assumed duplexes 3230 may be estimated using a subset of MAESTRO probes that poorly enrich the mutations, such that the probes show little bias for mutated versus wild-type alleles. As such, these sites may be used to estimate the number of DNA molecules per locus, which, when multiplied by the number of mutations assayed, may give the total molecules assayed. Additionally, or alternatively, the total assumed duplexes 3230 may be estimated by including a subset of control probes that are designed to not have bias for mutant versus wild-type DNA and may thus be used to estimate the number of DNA molecules per locus, which, when multiplied by the number of mutations assayed, may give the total molecules assayed. Together, the total mutated duplexes 3228 and the total assumed duplexes 3230 may be used to determine a tumor fraction, e.g., the proportion of reads that contain the mutation (e.g., the total mutated duplexes 3228) out of the total number of reads covering the mutation site (e.g., the total assumed duplexes 3230). The total mutated duplexes 3228 and the total assumed duplexes 3230 comprise sample-specific specificity factors, for instance.

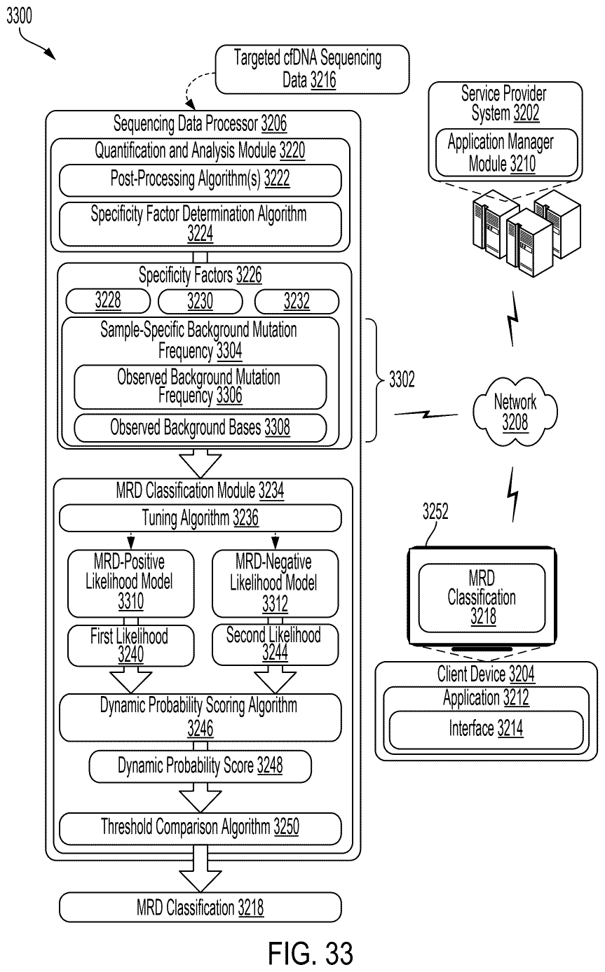

In the environment 3200, the specificity factors 3226 further comprise an assumed background mutation frequency 3232. As described herein, the assumed background mutation frequency 3232 may be a fixed value, a context-specific value, or a sample-specific value (e.g., an empirically measured value). The assumed background mutation frequency 3232 may refer to a background SNV frequency, for example, and may serve as an error rate. For instance, the assumed background mutation frequency 3232 may be used as the error rate mentioned above in the Dynamic MRD Calling section. As a non-limiting example of a fixed value, the assumed background mutation frequency 3232 may be 1 out of 10 million (e.g., 1×10⁻⁷), although other values may be used. It is to be appreciated that the specificity factors 3226 may include other factors in addition to or as an alternative to those listed above, including additional context-specific factors that will be described below with respect to FIG. 33.

In accordance with the techniques described herein, the sequencing data processor 3206 includes an MRD classification module 3234. The MRD classification module 3234 is representative of the functionality to determine whether a given sample is MRD positive or MRD negative (e.g., the MRD classification 3218) based on the targeted cfDNA sequencing data 3216 and the specificity factors 3226. In at least one variation, the MRD classification module 3234 is configured to determine whether the given sample is positive for circulating tumor DNA or negative for circulating tumor DNA. In one or more implementations, the MRD classification module 3234 includes a tuning algorithm 3236 that takes into account the specificity factors 3226 to tune parameters of an MRD-positive likelihood model 3238 (e.g., a first likelihood model) configured to output a first likelihood 3240 and an MRD-negative likelihood model 3242 (e.g., a second likelihood model) configured to output a second likelihood 3244.

The MRD-positive likelihood model 3238, for instance, determines a likelihood of the observed data given that the sample is MRD positive, such as according to Equation 2 given above. The MRD-positive likelihood model 3238 may use a binomial distribution to model the total mutated duplexes 3228 and the total assumed duplexes 3230 given a probability parameter t, where t ensures that the estimated mutation frequency does not fall below the assumed background mutation frequency 3232. The first likelihood 3240 may be a likelihood that the total mutated duplexes 3228 among the total assumed duplexes 3230 are exclusively derived from tumors.

The MRD-negative likelihood model 3242, for instance, determines a likelihood of the observed data given that the sample is MRD negative, such as according to Equation 3 given above. The MRD-negative likelihood model 3242 may use a binomial distribution to model the total mutated duplexes 3228 and the total assumed duplexes 3230 given the assumed background mutation frequency 3232. The second likelihood 3244 may be a likelihood that the total mutated duplexes 3228 among the total assumed duplexes 3230 are spontaneous errors.

The total mutated duplexes 3228 and the total assumed duplexes 3230 are used by the MRD-positive likelihood model 3238 and the MRD-negative likelihood model 3242 as sample-specific inputs, thus enabling the MRD classification module 3234 to adapt its confidence to the particular sample being evaluated.

The first likelihood 3240 and the second likelihood 3244 are input to a dynamic probability scoring algorithm 3246, which outputs a dynamic probability score 3248. By way of example, the dynamic probability scoring algorithm 3246 may use a posterior probability calculation, such as Equation 1 provided above, where P (M⁺|D) is the dynamic probability score 3248 that the sample is MRD positive given the observed data (D), P (D|M⁺) is the first likelihood 3240, and P (D|M⁻) is the second likelihood 3244. The dynamic probability scoring algorithm 3246 may further use priors, e.g., P (M⁺) and P (M⁻) of Equation 1, which may be fixed values (e.g., 0.5 as a non-limiting example).

The MRD classification module 3234 may further include a threshold comparison algorithm 3250 that is configured to output the MRD classification 3218 based on the dynamic probability score 3248 relative to a threshold. By way of example, the MRD classification 3218 may classify the sample as MRD positive in response to the dynamic probability score 3248 being greater than or equal to the threshold, whereas the MRD classification 3218 may classify the sample as MRD negative in response to the dynamic probability score 3248 being less than the threshold. As a non-limiting example, the threshold is 0.95. In at least one implementation, the threshold is adjustable, e.g., via user input.

The client device 3204 is shown displaying, via a display device 3252, the MRD classification 3218. By way of example, the display device 3252 may display an output indicating that the sample is MRD positive or MRD negative. It is to be appreciated that the MRD classification 3218 is also stored in memory, in a single data file or across multiple data files, for subsequent access.

In this way, the MRD classification module 3234 generates the MRD classification 3218 for increased circulating tumor DNA detection sensitivity and a decreased probability of false detection.

### MAESTRO Enrichment and Sequencing Requirements

MAESTRO enrichment and sequencing requirements followed a similar strategy as previous work. Duplex variant allele frequency (VAF), the ratio of mutated duplexes to total duplexes at a particular locus, was used to estimate mutation enrichment between MAESTRO and MRD Tracker. For this, sites were restricted to those that (1) were in both the MAESTRO and MRD Tracker fingerprints, (2) passed all filters with MAESTRO and MRD Tracker (described in Data Processing and Filtering) and (3) had ≥1 mutated duplex detected with both MAESTRO and MRD Tracker. This enabled direct comparisons between MAESTRO and MRD Tracker per site and MAESTRO's VAF fold enrichment to be easily calculated (MAESTRO VAF/MRD Tracker VAF). Similarly, for comparing sequencing requirements, sites were restricted to those that (1) were in both the MAESTRO and MRD Tracker fingerprints and (2) passed all filters with MAESTRO and MRD Tracker. Using these sites, each duplex family was downsampled in steps down to 0.01% to form saturation curves between total read pairs and mutated duplexes. The read pairs were compared to reach mutated duplex saturation by (1) restricting to samples that had ≥2 mutated duplexes detected with MAESTRO and MRD Tracker at sites in both fingerprints, (2) taking the minimum saturated mutated duplex count (saturation point was defined as the minimum mutated duplex count that was >90% of the total mutated duplex count or just the total mutated duplex count if the sample did not saturate) between MAESTRO and MRD Tracker as the comparison point, (3) finding the minimum number of read pairs for the mutated duplex count to reach the comparison point with MAESTRO and MRD Tracker, and (4) calculating the ratio of the read pairs with MAESTRO and MRD Tracker.

### Results

### Maestro-Pool and an Improved MRD Caller

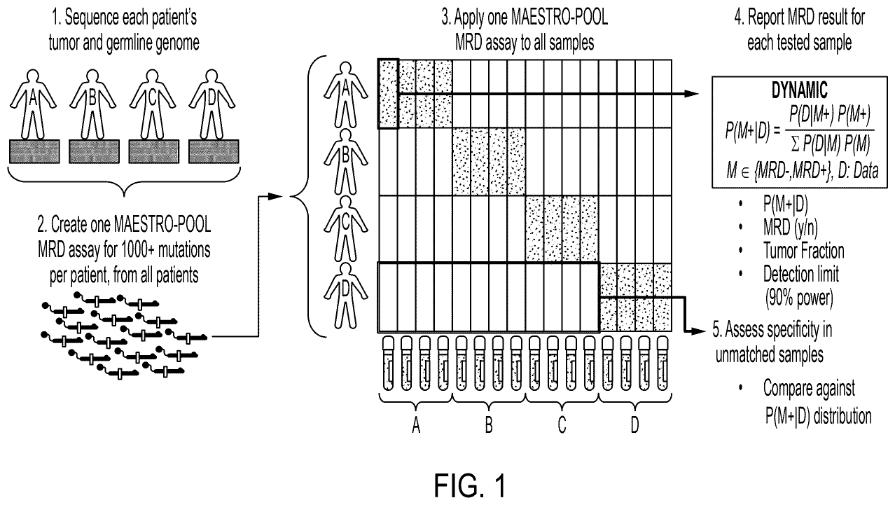

MAESTRO-Pool enables massively parallel, tumor-informed MRD testing for patient cohorts. It involves performing whole-genome sequencing (WGS) of each patient's tumor, designing MAESTRO probes that target patient-specific tumor mutations, and combining these into a single assay that is applied to all samples from all patients (FIG. 1). Mutations are verified in each patient's tumor DNA and excluded if found in their own germline DNA as previously described. MRD detection is performed for each patient's own plasma samples (i.e., “matched samples”) as well as plasma samples from other patients (i.e., “unmatched samples”) to assess specificity of each bespoke assay. Mutations shared in tumor biopsies from multiple patients are excluded when computing the specificity of each MRD test in unmatched samples.

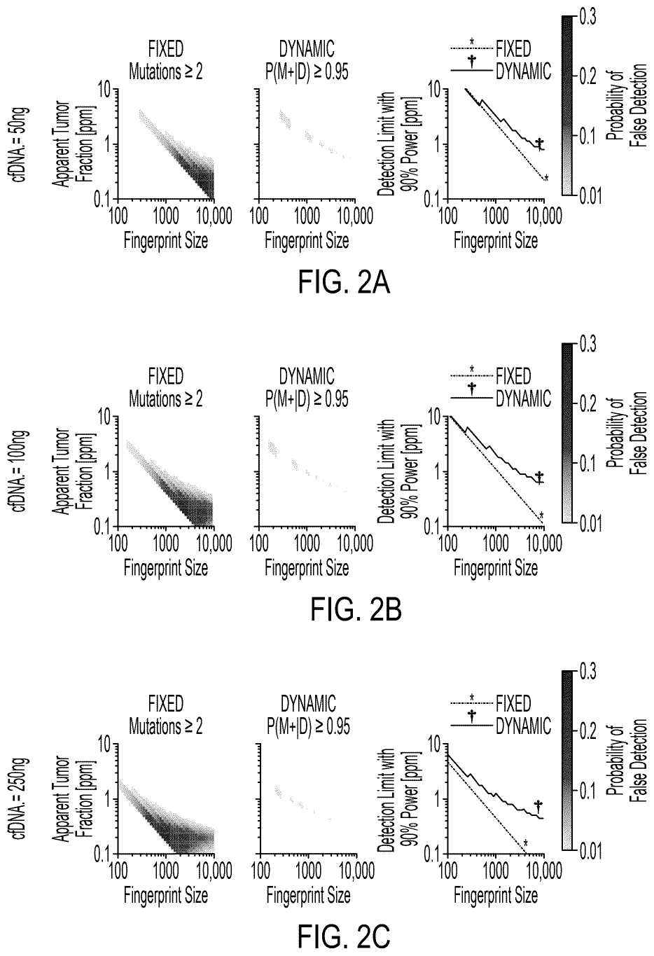

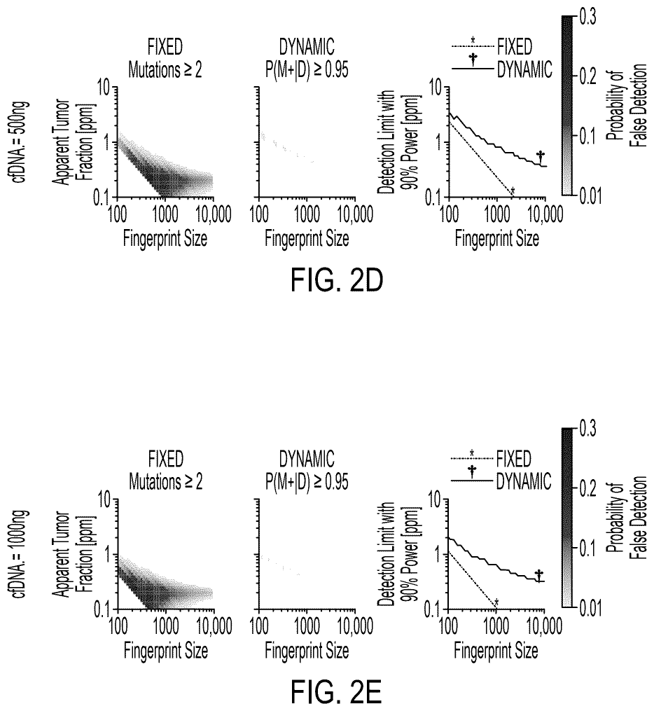

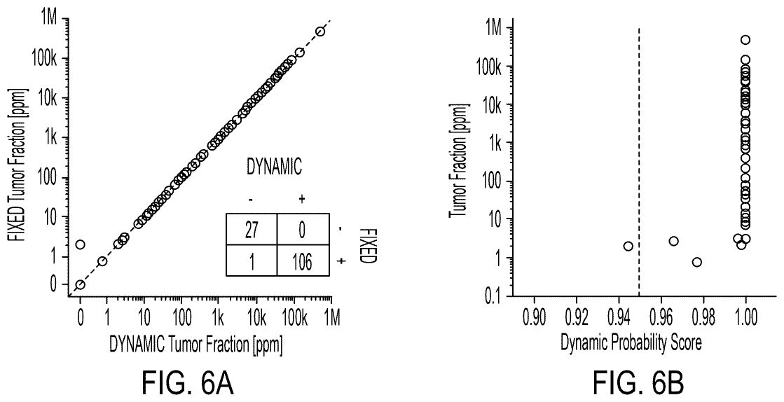

To this point, a “fixed” threshold has been used for MRD detection with MAESTRO of ≥2 mutated duplexes when assaying approximately 1000 genome-wide mutations from standard blood volumes (e.g., 1 to 3× 10 cc tubes). This has shown high specificity in benchmarking experiments and cancer patients, but it was reasoned that it may be insufficient to track even more mutations and cfDNA molecules per patient (FIGS. 2A-2E). Therefore, a new “dynamic” MRD caller was developed that computes a probability for MRD detection based on sample-specific attributes such as observed tumor fraction, fingerprint size, and cfDNA molecules, against an assumed error rate of 1/10 M (see Materials and Methods). A threshold of P≥0.95 was set—a tunable parameter—for a sample to be considered MRD positive. At P≥0.95, the model suggests this will limit false detection while maintaining high sensitivity down to low parts per million (ppm) levels of tumor-derived cfDNA (FIGS. 2A-2E). It was first confirmed that the dynamic caller had negligible impact on prior results (n=133/134 concordant MRD test results) when up to 1000 mutations per patient were assayed in up to 148 ng cfDNA with MAESTRO (FIGS. 6A-6B). Notably, the one sample with a discordant MRD call was borderline negative with dynamic MRD calling with a probability score of 0.94.

### Maestro-Pool Testing of Melanoma Patients

Next, it was sought to push the bounds on the number of mutations assayed, and used MAESTRO-Pool was used to experimentally validate the new MRD caller in matched vs unmatched samples. 9 patients treated with curative intent for Stage III melanoma were identified with a median of 10 (range 5 to 22) plasma samples each (98 total) collected over a median of 2.5 years (range 0.5 to 4.0) of clinical follow-up. Eight of the patients experienced recurrence, 7 of which had subsequent samples collected, while one patient remained disease-free but passed away due to unrelated circumstances. Whole genome sequencing (WGS) of tumor and normal DNA from each patient was performed and a median of 40,348 mutations (range 5932 to 160,598) per patient was identified. One MAESTRO-Pool assay for all patients was created (median 1856 mutations per patient, range 447 to 5571) and applied to each patient's tumor and normal DNA, as well as the 98 plasma DNA samples from all patients. For comparison, 9 personalized MRD Tracker assays were created, optimized from a previously validated method, comprising a median 1000 (range 334 to 1000) mutations per patient. These were capped at 1000 mutations per patient and only applied to each patient's own samples given the substantially greater sequencing requirements without MAESTRO enrichment.

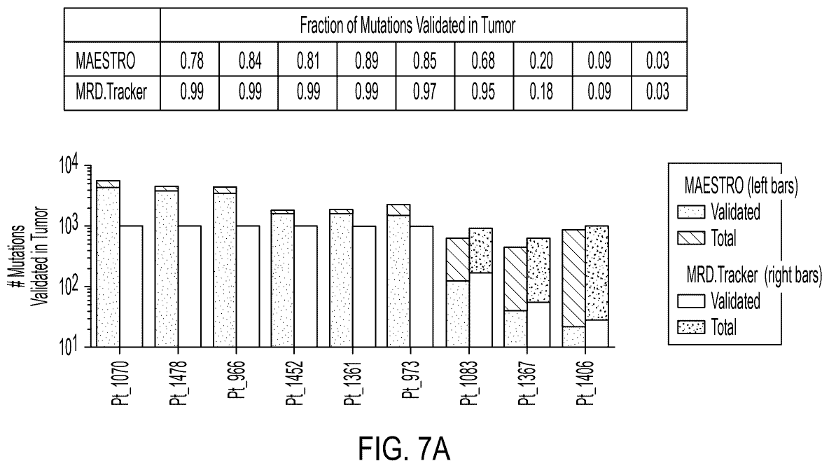

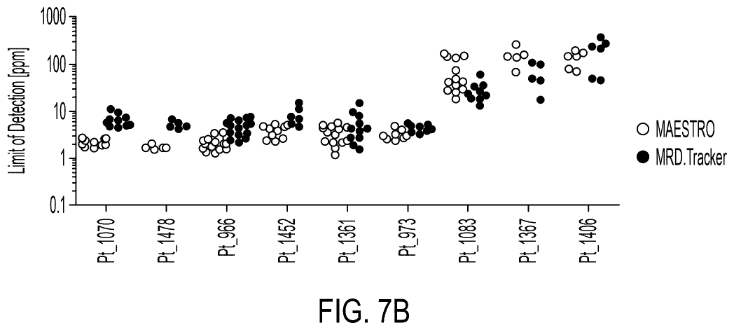

By applying MRD assays to each patient's tumor and normal DNA, each patient's tumor mutation fingerprint was first validated. With vs without MAESTRO enrichment, a median of 78% (range 3% to 89%) and 97% (range 3% to 99%) of mutations were confirmed, respectively (FIG. 7A). Expectedly, the specimens with the lowest validation rates had the lowest median variant allele frequency (VAF) in WGS, which is likely indicative of low tumor purity. Only the mutations that validated as present in the tumor biopsy and absent from the normal DNA were used for MRD detection from plasma (see Materials and Methods). Further, the estimated detection limits were computed at 90% power for each sample based on the duplex depths and validated fingerprint sizes (see Materials and Methods). Most MAESTRO tests were found to be powered to detect low ppm, except for the 3 patients with validated fingerprint sizes well below 1000 mutations (FIG. 7B).

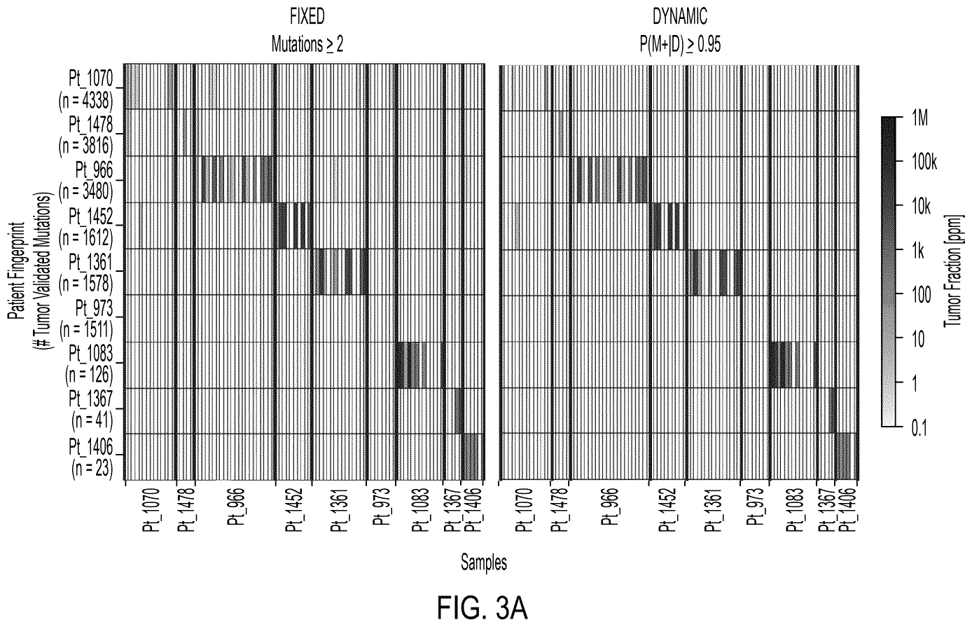

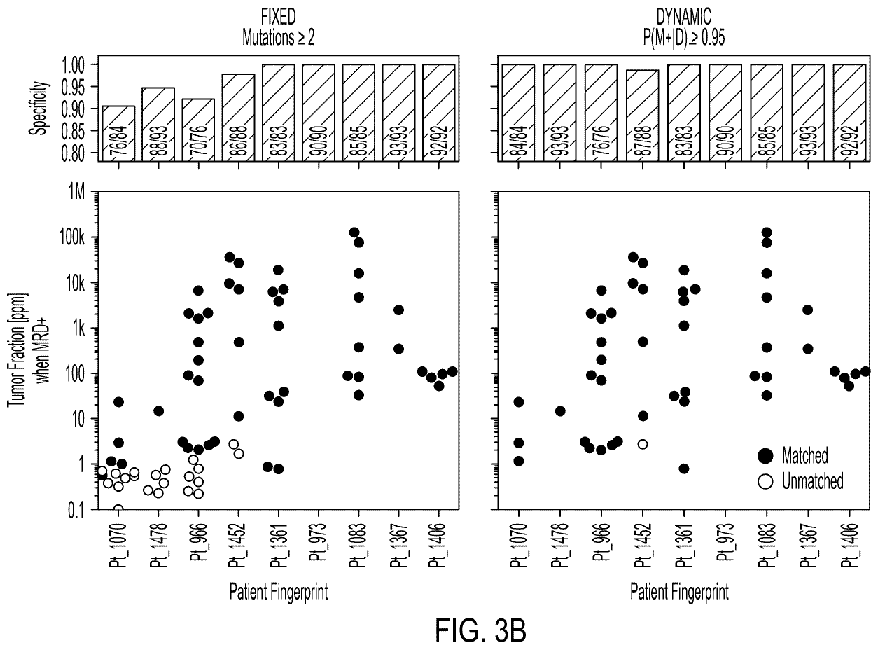

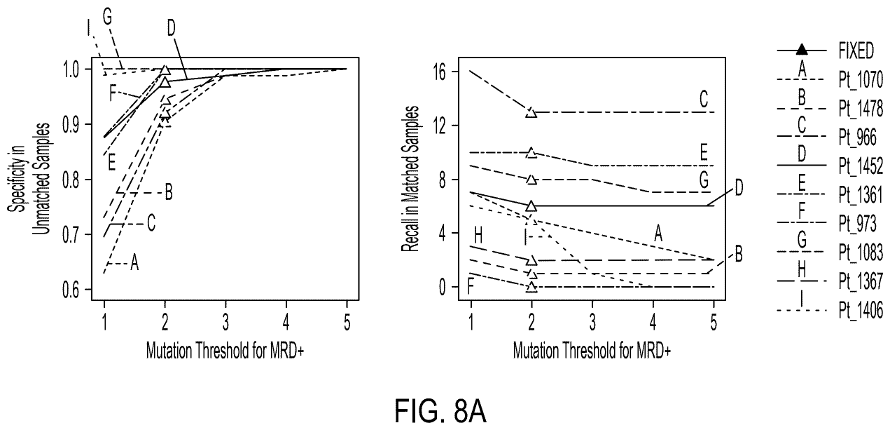

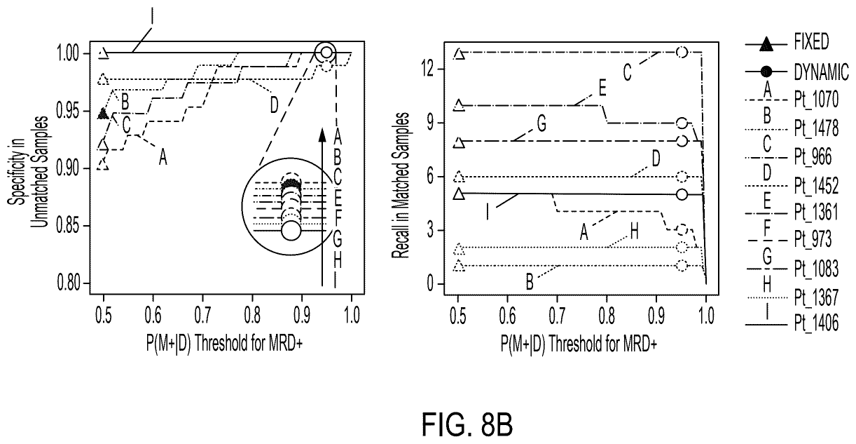

Applying the dynamic MRD caller with P≥0.95, 47 of 98 matched samples were found to be MRD positive (median tumor fraction 1.1×10⁻⁴, range 7.8×10⁻⁷ to 0.13) and only 1 of 784 unmatched samples to be MRD positive (tumor fraction 2.7×10⁻⁶, FIGS. 3A-3B). This resulted in median experimental specificities of 100% (range 100% to 100%) and 100% (range 98% to 100%) for detecting ≥10 ppm and <10 ppm ctDNA, respectively, in a median of 88 (range 76 to 93) unmatched patient samples. Of note, had a fixed threshold of ≥2 mutations been used, an additional 3 matched and 20 unmatched samples would have been found MRD positive with most false detection occurring in the largest panels of 4337, 3816, and 3479 mutations, as expected (FIGS. 3A-3B). While a stricter threshold of ≥3 or ≥4 mutations achieved specificities comparable to dynamic MRD calling, it also reduced the number of MRD positive calls in matched samples (FIG. 8A). These results highlight the importance of dynamic MRD calling as more tumor mutations and cfDNA molecules are sequenced per patient.

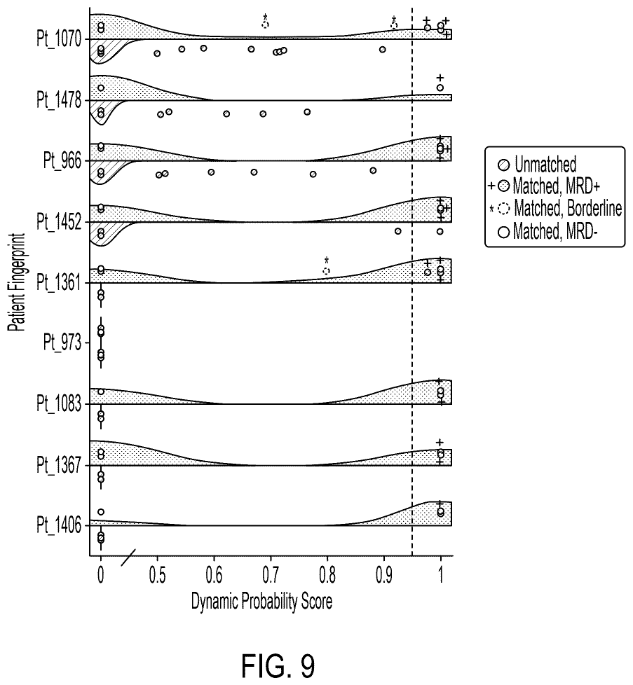

Interestingly, of the 3 samples that were MRD positive with fixed MRD calling but negative with dynamic calling, all had 2 or 3 mutations detected and tumor fractions below 1 ppm (range 5.7×10⁻⁷ to 9.9×10⁻⁷) suggesting that these were borderline MRD calls initially. This is further reflected in their dynamic probability scores ranging between 0.69 and 0.92. Although dynamic MRD calling successfully reduced the false detection rates, the P≥0.95 threshold was quite conservative (e.g., median specificity of 1). Tuning of this parameter was explored. It was observed that the predicted specificity underestimated the measured specificity and, therefore, lower probability thresholds, such as P≥0.80, still yielded higher specificity and comparable recall to fixed MRD calling (FIG. 8B). Additionally, the dynamic probability score enabled the comparison of MRD signal between samples while correcting for sample-specific attributes. This can be leveraged to quantify a confidence score by comparing the probability score of matched samples to the probability score distribution in unmatched samples. For example, matched samples with borderline-negative MRD signals (i.e., dynamic scores of 0.50≤P<0.95) can be assigned a confidence score by quantifying the fraction of unmatched samples with a probability score greater than or equal to their own (FIG. 9). This could further inform whether there may be traces of MRD in borderline-negative samples.

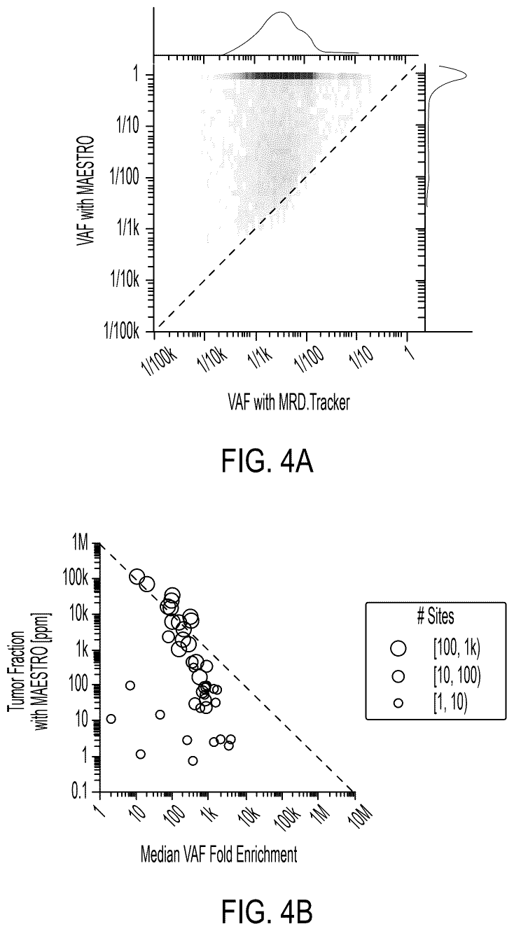

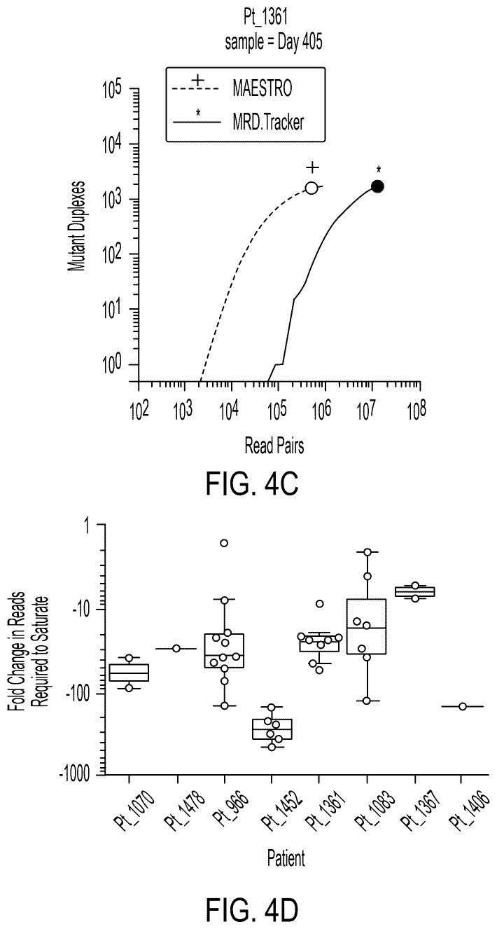

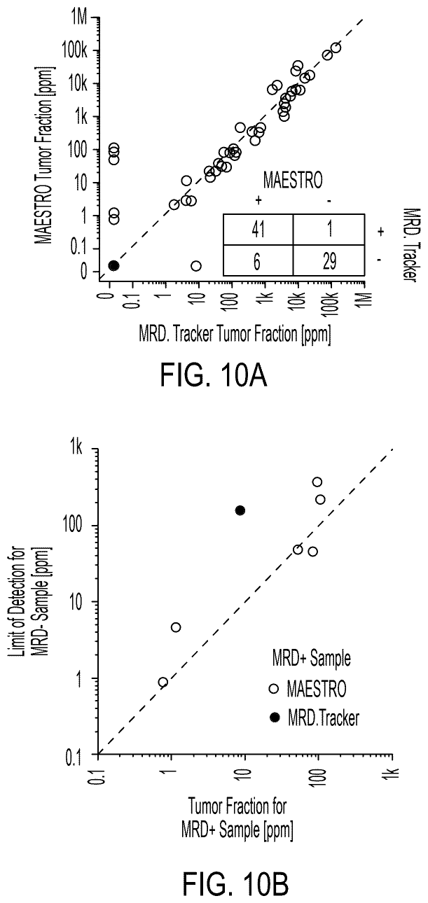

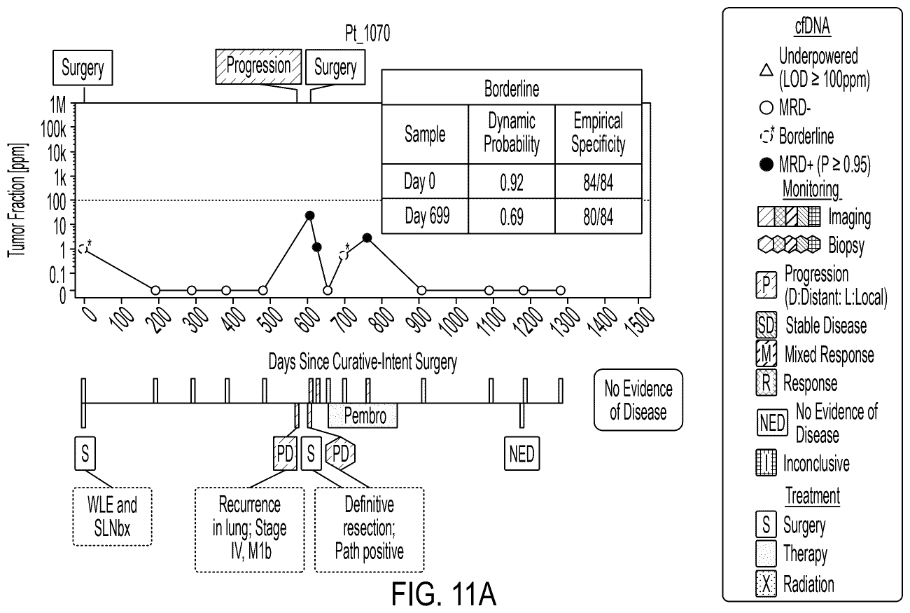

As further validation, MAESTRO-Pool results were compared using the dynamic caller for matched patient samples against the MRD results with MRD Tracker. For MRD Tracker, a standard cutoff of ≥2 mutations detected was used. High concordance in MRD detection was observed—70 of 77 samples with concordant MRD calls—and corresponding tumor fractions (r²=0.97) in cfDNA between MAESTRO-Pool and MRD Tracker (FIG. 10A). Of the 7 samples with discordant MRD calls, 5/7 of the MRD positive samples had observed tumor fractions below the detection limit of the MRD negative samples (FIG. 10B) and 4/7 of the samples were from patient 1406, which only had 22 and 28 tumor-validated mutations with MAESTRO-Pool and MRD Tracker, respectively. This suggested that the discordant samples were caused by one of the samples being underpowered. Reassuringly, of the 30 samples that were negative for MRD by MAESTRO, 29 were also negative for MRD with MRD Tracker.

Lastly, the effect of MAESTRO enrichment on sequencing efficiency was explored. For mutations tracked with MAESTRO-Pool and MRD Tracker, a median 115-fold enrichment was found (range 0.44 to 12 056) in VAF when using MAESTRO (FIG. 4A). Expectedly, the fold-enrichment was most substantial among samples with low tumor fractions, e.g., median 777 (range 1.3 to 8631) for samples with <100 ppm tumor DNA (FIG. 4B). Next, the reads for uncovering mutated DNA duplexes with MAESTRO-Pool vs MRD Tracker was examined on a sample-by-sample basis (FIGS. 4C-4D). A median 33-fold reduction in reads (range 1.5 to 439) was found to uncover 90% of the mutated DNA duplexes in each sample using MAESTRO (FIG. 4D). The results using MAESTRO-Pool are consistent with prior observations that MAESTRO enables highly sensitive MRD detection using substantially less sequencing.

### Case Studies of Individual Melanoma Patients

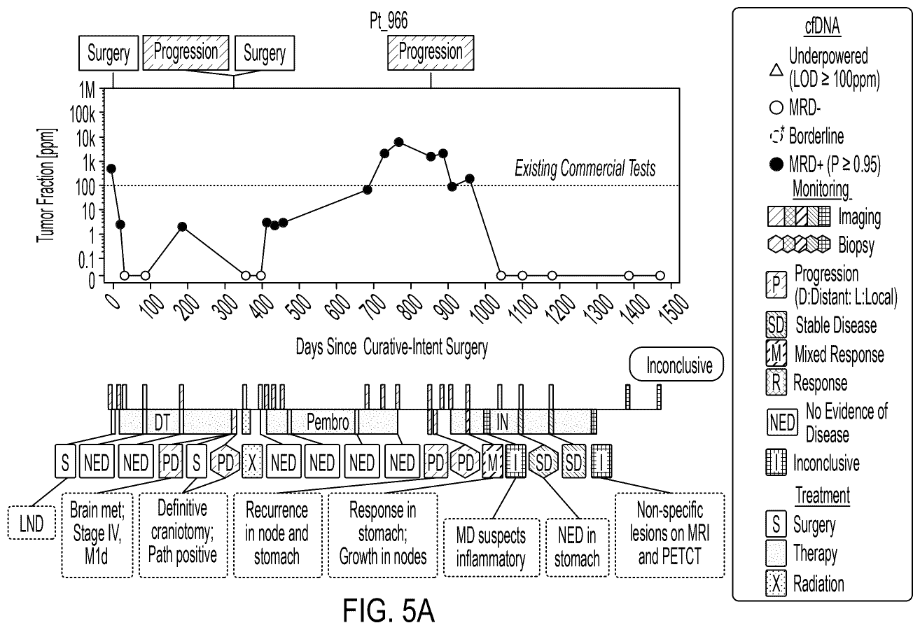

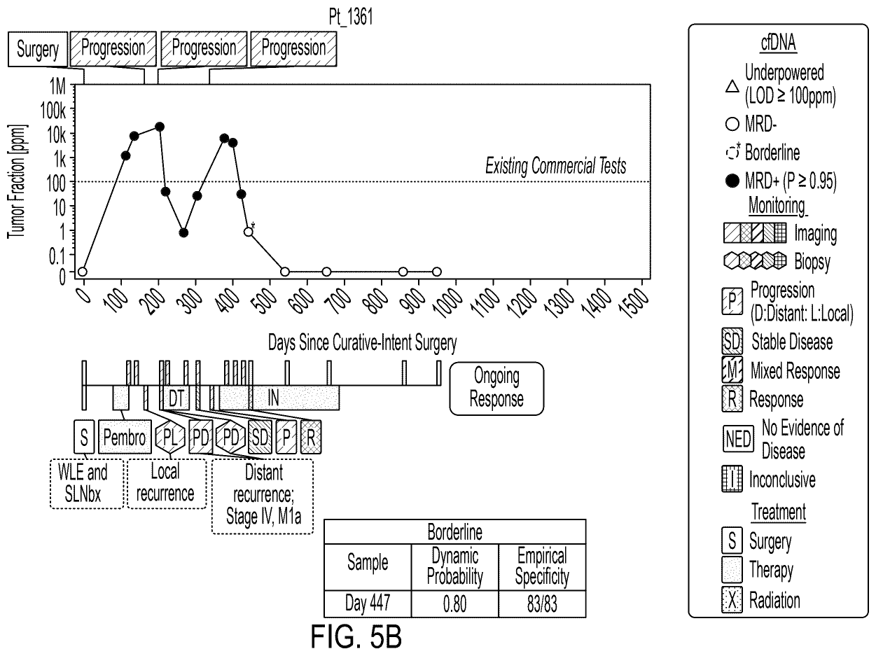

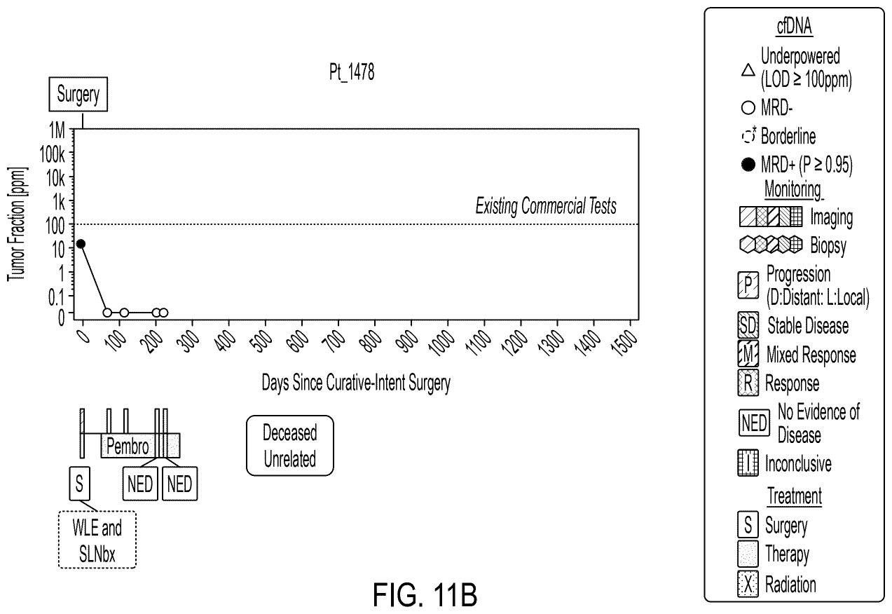

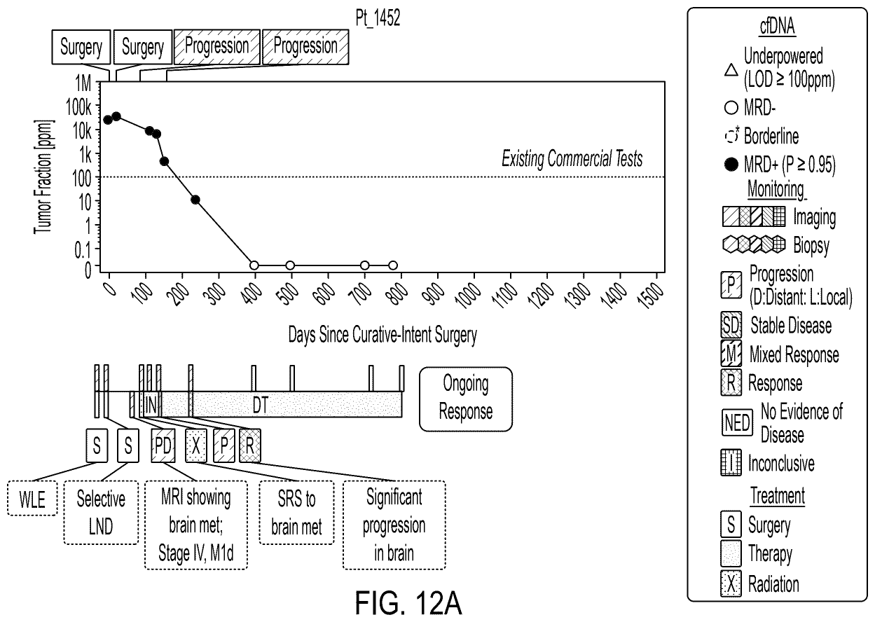

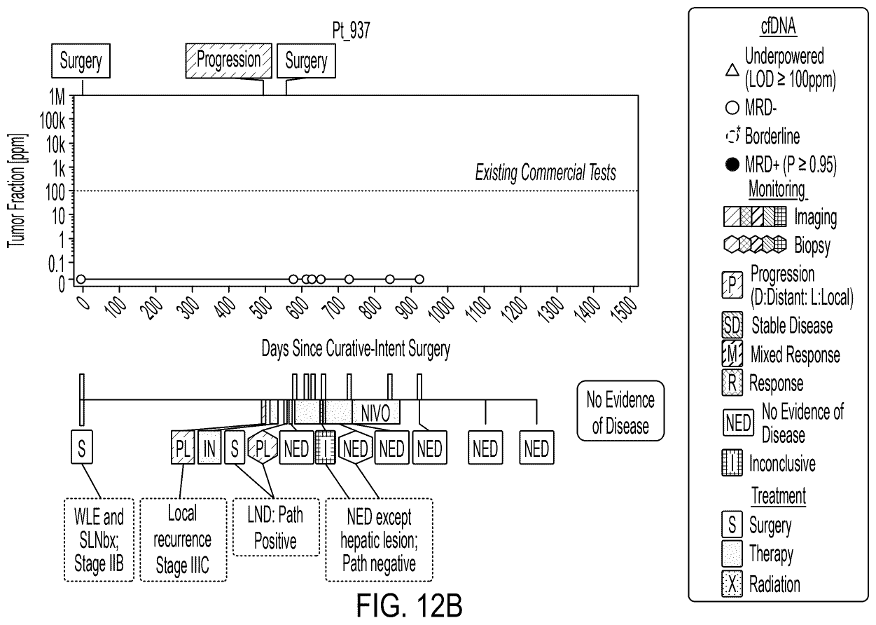

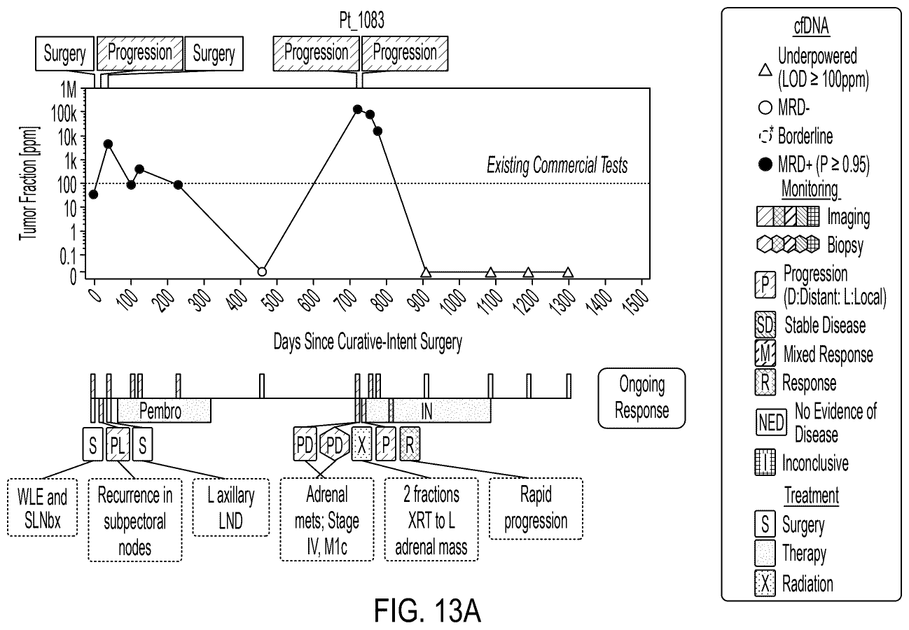

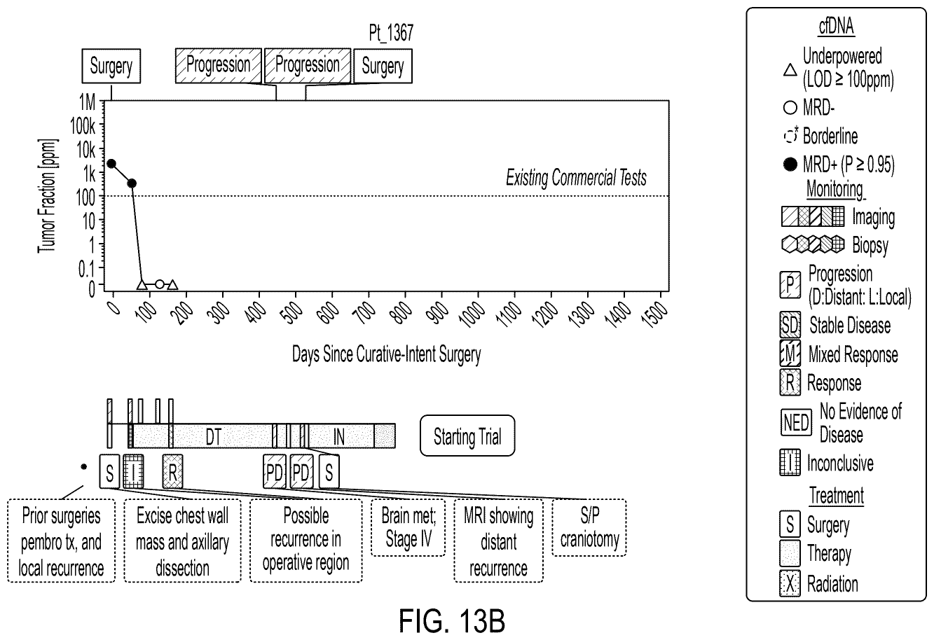

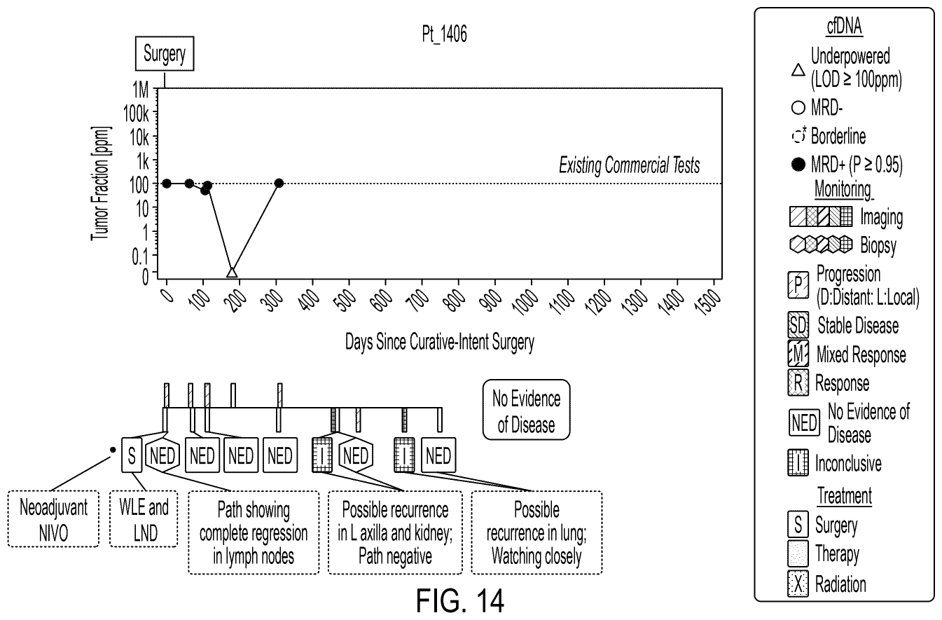

Finally, how the MRD testing results related to the treatments and outcomes of individual patients was examined. As this was a cohort study and not a clinical trial, there was wide variation in treatment and follow-up for individual patients, lending each to be examined individually (FIGS. 5A-5B, FIGS. 11A-11B, FIGS. 12A-12B, FIGS. 13A-13B, and FIG. 14). In one exemplary patient, MRD was detected following surgery at 2.6 ppm immediately prior to adjuvant dabrafenib and trametinib therapy (FIG. 5A). MRD then became undetectable at 2 subsequent time points before rising to 2.1 ppm 138 days before recurrence was detected in the brain. Following definitive craniotomy and radiation treatment, MRD was undetectable at two time points. Then, immediately before pembrolizumab, MRD was detected at 3.1 ppm. At two time points thereafter, it remained at similar levels. The patient continued therapy for another 355 days until MRD levels rose to 6612 ppm and a gastric metastasis was detected 442 days after initial detection. Then, the patient was given ipilimumab and nivolumab, and MRD became undetectable while imaging scans were inconclusive.

In a second exemplary patient, there were no samples available shortly after surgery, but MRD was detected 48 and 84 days preceding both local and distant recurrence, respectively (FIG. 5B). MRD levels subsequently declined down to 0.8 ppm before rising again. Of note, an increase in MRD from 0.8 to 24 ppm was detected when imaging scans indicated stable disease and 35 days before scans showed progression. The patient was then given ipilimumab and nivolumab and showed a stark decline in MRD levels at least down to 0.9 ppm preceding a long and durable response that was ongoing at last follow-up. These cases exemplify the deep levels of MRD that can be detected with MAESTRO and suggest the potential for using these measurements to guide care.

Interestingly, of the 8 MRD positive samples with ultra-low tumor fractions (<10 ppm) detected among all patients in the cohort, 7 were found either preceding recurrence (in an upward trend of increasing tumor fraction) or after a decline in tumor fraction leading to a period of therapy response. Notably, 4 of these samples occurred at time points for which patients had no evidence of disease via imaging scans. These observations, together with the experimental specificities, suggest that deep detection of MRD could fill meaningful voids in the monitoring of cancer treatment response.

### Discussion

Liquid biopsies hold tremendous promise for detecting MRD and informing more precise cancer care but would benefit from higher sensitivity. Assaying more mutations in more cfDNA could improve sensitivity using bespoke tests, but this creates opportunities for false detection if not properly accounted. Here, MAESTRO-Pool is introduced for massively parallel, tumor-informed MRD testing in cohort studies. By pooling multiple patients' MRD tests together, MAESTRO-Pool assays thousands of patient-specific tumor mutations while concurrently benchmarking each patient's bespoke MRD test using unmatched samples as controls. This has streamlined MRD testing and enabled the detection of low ppm levels of ctDNA in patients with melanoma, including when scans showed no evidence of disease. Additionally, the new dynamic MRD caller accounted for differences in the numbers of mutations and cfDNA molecules sequenced by computing a probability score for MRD in each sample. Using the dynamic MRD caller with MAESTRO-Pool, median experimental specificities of 100% (range 100% to 100%) and 100% (range 98% to 100%) were observed for detecting ≥10 ppm and <10 ppm ctDNA, respectively, when each patient's test was benchmarked in a median of 88 unmatched samples (range 76 to 93) from other patients. The ability to identify borderline-negative patients who may benefit from follow-up testing was demonstrated using the dynamic caller and its probability score distribution in pooled testing.

The massive excess of normal cfDNA in blood and the large number of sequencing reads used to overcome sequencing errors often prohibits the routine benchmarking of individualized MRD assays in control samples. The challenge is further compounded when more mutations and more cfDNA are assayed to enhance MRD detection at key clinical time points. MAESTRO-Pool addresses these challenges by leveraging mutation enrichment to reduce the amount of sequencing required per sample and allow multiple patients' MRD tests to be pooled and applied to many samples at once. A similar approach would be impractical using standard duplex sequencing approaches, as it would be significantly slower and financially inviable, both of which would ultimately burden healthcare systems and patients. Meanwhile, the dynamic caller further accounts for varied numbers of mutations and cfDNA molecules to limit false detection. Of note, the dynamic caller works with both MAESTRO and MAESTRO-Pool MRD testing.

In summary, MAESTRO-Pool and dynamic MRD calling enabled ctDNA detection down to 1 ppm while simultaneously benchmarking each patient's bespoke MRD test using unmatched patient samples as controls. These features streamlined MRD testing and enabled higher sensitivity and specificity as more mutations and cfDNA were analyzed. In turn, these approaches for enhanced MRD detection stand to enable more precise care for cancer patients.

### Example 2. Impact of Higher Cell-Free DNA Yields on Liquid Biopsy Testing in Glioblastoma

Minimally invasive molecular profiling using cell-free DNA (cfDNA) is increasingly important to the management of cancer patients; however, low sensitivity remains a major limitation, particularly for brain tumor patients. Transiently attenuating cfDNA clearance from the body—thereby, allowing more cfDNA to be sampled—has been proposed to improve the performance of liquid biopsy diagnostics. However, there is a paucity of clinical data on the effect of higher cfDNA recovery. Here, the impact of collecting greater quantities of cfDNA on circulating tumor DNA (ctDNA) sensitivity in the ‘low shedding’ cancer type glioblastoma was investigated by analyzing up to ~15-fold more plasma than routinely obtained clinically.

Seventy plasma samples (median 17.0 mL, range 2.5-66.5) from 8 IDH-wildtype glioblastoma patients were tested using an optimized version of the MAESTRO-Pool ctDNA assay. Results were compared with simulated single-blood-tube equivalents of cfDNA. ctDNA results were then compared with MRI and pathology assessments of true progression vs. pseudo-progression in glioblastoma patients.

Larger cfDNA yields exhibited a doubling in ctDNA-positivity while achieving a median specificity of 99% and more precise ctDNA quantification. In 8 glioblastoma patients, ctDNA was detected in 88%, including at multiple timepoints in 6/7. In the setting of indeterminate progression by MRI, the data suggested that MAESTRO-Pool with large plasma volumes can help distinguish true glioblastoma progression from pseudo-progression.

These findings provide a proof-of-principle that most glioblastomas shed ctDNA into plasma and that greater ctDNA yields could help improve liquid biopsies for “low shedding” cancer types such as glioblastoma.

### Importance of the Study

Liquid biopsies hold promise for improving cancer detection and monitoring, but are limited by the scarcity of circulating tumor DNA (ctDNA) in blood. IDH-wildtype glioblastoma is considered a “low shedder” for which ctDNA is infrequently detected by current technologies. The ultrasensitive MAESTRO liquid biopsy was optimized for large plasma volumes (up to 66.5 mL, ~15× the amount in a typical blood draw) from glioblastoma patients. Larger cfDNA yields exhibited a doubling in ctDNA-positivity compared to typical plasma volumes, while achieving a median specificity of 99%. ctDNA was detected in 7/8 of glioblastoma patients, including at multiple timepoints in 6/7. Compared to MRI and pathology, ctDNA from larger plasma volumes helped improve the sensitivity and specificity of glioblastoma progression vs. pseudo-progression determination. These findings provide a proof-of-principle that most glioblastomas shed ctDNA into plasma and that greater ctDNA yields could help improve liquid biopsies for “low shedding” cancers such as glioblastoma.

### Introduction

Advances in cell-free DNA (cfDNA) diagnostics have enabled the minimally invasive diagnosis of cancer, identification of therapeutic targets and resistance, and monitoring of minimal/measurable residual disease (MRD)—all from a simple blood draw. Yet, higher sensitivity is needed in many cancer contexts in which plasma circulating tumor DNA (ctDNA) levels are frequently less than 0.01-0.1% of total cfDNA. To enhance the sensitivity of ctDNA tests, efforts have primarily focused on tracking more somatic variants and integrating other features such as DNA methylation or fragmentation patterns. However, at such low levels of ctDNA, stochastic sampling of the tumor genome in a blood draw limits the ability of such assays to sensitively detect, monitor, and genotype cancer from ctDNA. To help overcome these challenges, transiently attenuating cfDNA clearance from the body was recently proposed as a way to recover more cfDNA from a patient's blood and thereby improve liquid biopsy testing. Specifically, two complementary methods were previously developed: a monoclonal antibody against DNA and liposomes that can transiently reduce cfDNA clearance. While both approaches improved cancer detection sensitivity in mice, there is a paucity of clinical data regarding the impact of higher cfDNA yields on liquid biopsy performance due to constraints on blood volumes typically collected in clinical settings.

To answer this pressing question, herein the bespoke (i.e., tumor-informed) MAESTRO-Pool MRD assay in Example 1 was further optimized to investigate the role that higher cfDNA yields from larger plasma volumes could have on tumor-informed ctDNA detection. The MAESTRO (Minor Allele Enriched Sequencing Through Recognition Oligonucleotides) method, as applied in Example 1 and this Example 2, is uniquely suited for analyzing large volumes of blood due to its ability to deplete the massive excess of normal cfDNA in blood and therefore, dramatically reduce sequencing costs. As described herein, with MAESTRO-Pool, multiple patients' MAESTRO assays can be pooled together into a single assay that is applied to all patients' samples, thereby enabling simultaneous detection of MRD and assessment of specificity using unmatched patient samples as controls.

As an especially informative setting for this study, a cohort of isocitrate dehydrogenase (IDH)-wildtype glioblastoma patients enrolled in an ongoing cancer vaccine trial (NCT02287428) was the focus given several of its unique characteristics. First, glioblastoma is an aggressive primary brain tumor that afflicts 17 people/million annually with a median survival of just 12-17 months. At the time of symptomatic presentation, glioblastoma can be found extensively infiltrating microscopically into the brain. As a consequence, even though surgical resection and chemo-radiotherapy may treat the tumor that is visible on imaging, the remaining neoplastic cells invariably lead to tumor progression. Second, the blood-brain barrier and distinct drainage mechanisms of the brain's interstitial fluid are thought to contribute to glioblastoma's classification as a ctDNA “low shedding” cancer type for which the sensitivity of existing plasma-based ctDNA assays has been relatively low. Third, the cohort was enrolled on a clinical trial that included serial large-volume peripheral blood collections with rapid banking of plasma, providing a wide range of cfDNA yields for analysis. Finally, contrast-enhanced magnetic resonance imaging (MRI)—the current gold standard for glioblastoma surveillance and response assessment—is often limited by low clinical sensitivity and specificity. Notably, although MRI is used for assessing glioblastoma tumor progression, it is complicated by the fact that treatment effect, inflammatory response, and radio-necrosis can all display similar radiographic features as tumor progression (i.e., pseudo-progression). Therefore, diagnostics that can distinguish radiographic pseudo-progression from bona fide tumor progression are urgently needed for glioblastoma patients. Surgical pathology can definitively diagnose tumor progression vs. pseudo-progression, but repeat surgery is often not feasible due to glioblastoma's refuge among delicate brain structures, underscoring the need for minimally invasive techniques to assess the tumor longitudinally. To improve the performance of the MAESTRO-Pool MRD assay in this setting, a new MRD detection algorithm was developed that incorporates sample- and context-specific background single nucleotide variant (SNV) frequencies into the dynamic MRD calling algorithm described in Example 1. This study shows that higher cfDNA yields could improve ctDNA testing in a low shedding cancer.

### Methods

### Patients and Samples

Patient tissue and blood samples were collected as part of a clinical trial (NCT02287428) while the ctDNA analyses were performed separate to the scope of the trial aims. A requirement for enrollment in this clinical trial was a gross total resection of tumor, which produced a uniform baseline across the eight patients where there was no gross evidence of tumor. Written informed consent was provided by all patients for the collection and analysis of tissue samples, blood samples, and clinical data using Dana-Farber/Harvard Cancer Center Institutional Review Board-approved protocols and in accordance with the Declaration of Helsinki.

Tumor samples were formalin-fixed and paraffin embedded (FFPE) following surgery. Peripheral blood samples that were used as a source of germline genetic information were frozen at the time of collection. Peripheral blood samples from which plasma was obtained were collected in either K₂ EDTA or BCT Streck® tubes and processed within 4 hrs. Plasma was carefully separated from peripheral blood by centrifugation at room temperature for 15 min at 1,500-1,800 g; followed by a second centrifugation at ≥2,500 g for 10 mins at room temperature. Plasma was stored at −80 °C until thawing for processing. For 1 of 8 patients (GBM_7), plasma samples were not available from their first 450 days of tumor treatment. For the remaining 7 patients, plasma samples were collected throughout their treatment course. As part of the clinical trial protocol, patients underwent MRIs at approximately 1-3 month intervals, with confirmation of tumor progression vs. pseudo-progression by surgical pathology as clinically indicated. Presence of tumor progression on MRI and pathology, as clinically determined by the patient's neuro-radiologists, neuro-oncologists, and neuropathologists, was abstracted with blinding to the MRD results using the criteria described in Table 1.

### Whole Genome Sequencing (WGS) of Tumor and Normal Samples

To identify tumor-specific mutations, tumor and normal genomic DNA was extracted from FFPE tissues and peripheral blood, respectively, at the Broad Institute's Genomic Platform, and WGS of tumor DNA (60× average coverage) and normal DNA (15× average coverage) was performed using NovaSeq™ S4. From the tumor and normal WGS data, somatic variants were called using the GATK Best Practices Mutect2 workflow. MAESTRO fingerprints and MRD Tracker fingerprints were designed as described herein.

### Plasma Sample Processing and Liquid Biopsy Assays

cfDNA was extracted from plasma using the QIAsymphony® Circulating DNA kit and quantified using the Quant-iT™ PicoGreen™ assay on a Hamilton® STAR-line liquid handling system. cfDNA libraries were constructed using the Kapa™ Hyper Prep kit with custom dual index duplex UMI adapters (IDT), as previously described. The prepared libraries were then quantified using the Quant-iT™ PicoGreen™ assay on a Hamilton® STAR-line liquid handling system. MAESTRO-Pool and MRD Tracker were performed following the methods described in Example 1. Details are provided in the Additional Methods for Example 2.

### MRD Data Analysis

The returned FASTQs were first aligned, deduplicated, and recalibrated by following the GATK Best Practices “Data pre-processing for variant discovery” workflow. Next, UMIs were extracted using fgbio, raw sequencing reads were converted into duplex consensus molecules, and duplex molecules were subjected to fragment-level and site-level filters as described herein. Notably, data were restricted to tumor validated sites that had ≥1 and 0 mutated duplexes in the patient-matched tumor and normal, respectively. One change to the MAESTRO-Pool processing workflow was the addition of a site-level outlier filter, which was added due to several low tumor fraction samples containing a site with an uncharacteristically high number of mutated duplexes. An outlier filter for low tumor fraction samples (≤10 ppm) was implemented because only 0 or 1 ctDNA molecules per site were expected in low tumor fraction regimes. To safeguard against possible false positives, each site in low tumor fraction samples was subjected to the following binomial test and with a threshold p-value≥0.01 (with multiple hypothesis correction):
Bin(ALTᵢ, Dᵢ, TFx_{LOO})≥0.01/n
   ALTᵢ: number of ALT duplexes at site i
   Dᵢ: estimated duplex depth at site i
   TFx_{LOO}: Sample's tumor fraction excluding site i (leave-one-out)
   n: number of tumor validated sites in fingerprint

In Example 2, only duplex molecules passing all applicable filters were considered for downstream analyses, including limit of detection estimation, MRD calling and tumor fraction estimation. These methods followed protocols described in Example 1 and Example 2. Details regarding the estimation of sample-specific SNV frequencies and the noise tuning for the Dynamic MRD caller are provided in the Additional Methods for Example 2.

Predicted MRD Results from Single-Blood-Tube Equivalents

In order to assess the impact of large plasma volume collection on MRD results, MAESTRO results were downsampled in silico, simulating the expected MRD results from typical plasma collection. 4 mL was selected to represent typical plasma collection assuming that a typical blood draw is about 10 mL with ~40% plasma yield. From this, each sample's normalizing factor was calculated based on its plasma volume, and the normalizing factor was used to perform random, binomial sampling of the observed duplexes per site. The downsampled duplexes were then used to call MRD and estimate LOD95 and tumor fractions. This process was repeated 50 times for each sample to obtain a distribution of random downsamplings. The predicted LOD95 was the median LOD95 from random downsamplings and the predicted tumor fraction (and error bars) was the median tumor fraction (and error bars) from the MRD-positive downsamplings. Lastly, the distribution was used to estimate the MRD likelihood by calculating the proportion of downsamplings that were called MRD-positive.

### Results

Greater cfDNA Yields from Larger Plasma Volumes Improve the Analytical Sensitivity of ctDNA MRD Testing

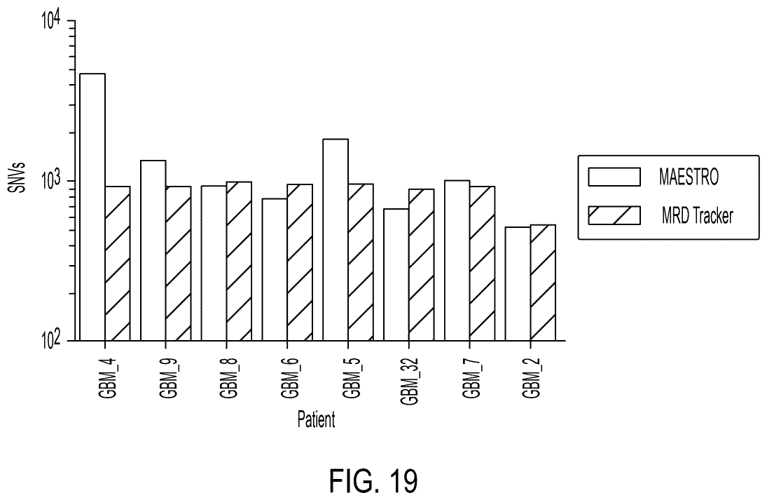

To investigate the role of larger plasma volumes, and thus higher cfDNA yields, on the analytical sensitivity of ctDNA MRD testing, 70 plasma samples collected from 8 glioblastoma patients (median 8 per patient, range 4-13) were evaluated. Plasma samples with known volumes were classified as either typical volume (i.e., <10 mL that would be obtained with 1-2 blood collection tubes; n=30, median 7.35 mL, range 2.5-9.7 mL) or large volume (i.e., ≥10 mL; n=32, median 34.0 mL, range 14.0-66.5 mL). Based on whole-genome sequencing of each patient's tumor and germline, tumor-specific SNVs were identified, from which bespoke mutation enrichment MAESTRO assays were designed. The MAESTRO assay targeted a median of 1,225 tumor-specific SNVs per patient (range 726-5,007; FIG. 19). Each of the eight patients' MAESTRO assays were then combined into one pooled assay called MAESTRO-Pool, as described herein in Example 1, targeting a total of 13,505 SNVs. MAESTRO-Pool was applied to all plasma samples from all patients. MAESTRO-Pool enables simultaneous MRD detection and specificity evaluation for bespoke assays by evaluating each sample with its patient-matched tumor target panel (i.e., fingerprint) or patient-unmatched tumor fingerprints, respectively. For orthogonal validation, bespoke MRD Tracker assays were used, which do not involve mutation enrichment sequencing (FIG. 19) and only applied to each patient's own plasma samples due to their high sequencing requirements.

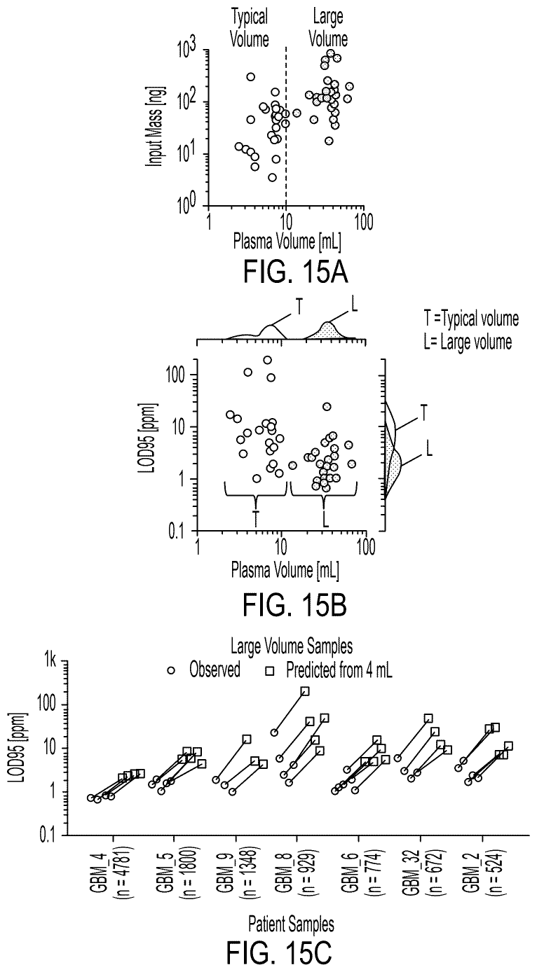

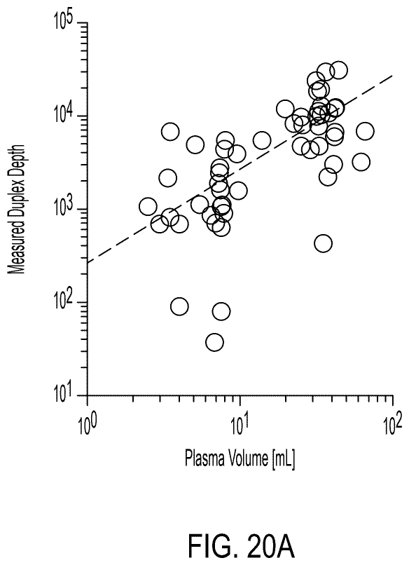

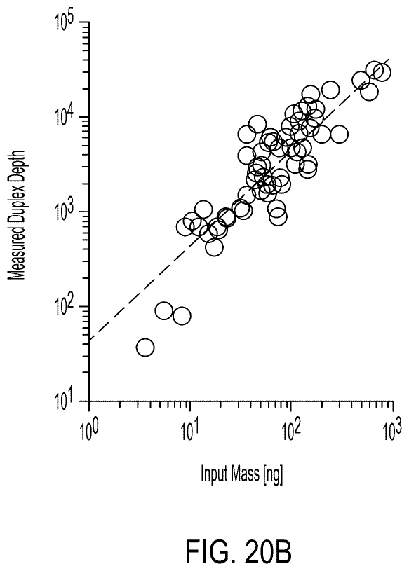

A correlation was observed between cfDNA yield and plasma volume (Pearson r=0.42, p=7.5×10⁻⁴, FIG. 15A). Furthermore, the measured duplex depth (i.e., the average number of DNA duplexes that could be recovered per locus) was also associated with plasma volume (Pearson r=0.54, p=5.6×10⁻⁶; FIG. 20A) and cfDNA input (Pearson r=0.88, p=2.3×10⁻²³; FIG. 20B). Based on the total duplexes that were evaluated per matched tumor fingerprint, the limit of detection (i.e., tumor fraction with 95% power [LOD95]) was estimated for all samples, and it was found that larger volume samples have lower LOD95s (median LOD95=1.9 parts-per-million (ppm), range 0.7-24.1 ppm) than typical volume samples (median LOD95=5.9 ppm, range 1.0-190.3 ppm; Mann-Whitney U p=2.3×10⁻⁶; FIG. 15B). By contrast, if only single-blood-tube equivalents (i.e., 4 mL of plasma; Methods) were collected, LOD95s would have been a median 5.0-fold (range 2.4-11.9) higher (FIG. 15C).

### Sample- and Context-Specific Noise Tuning Increases the Specificity of MRD Calling in MAESTRO Assays

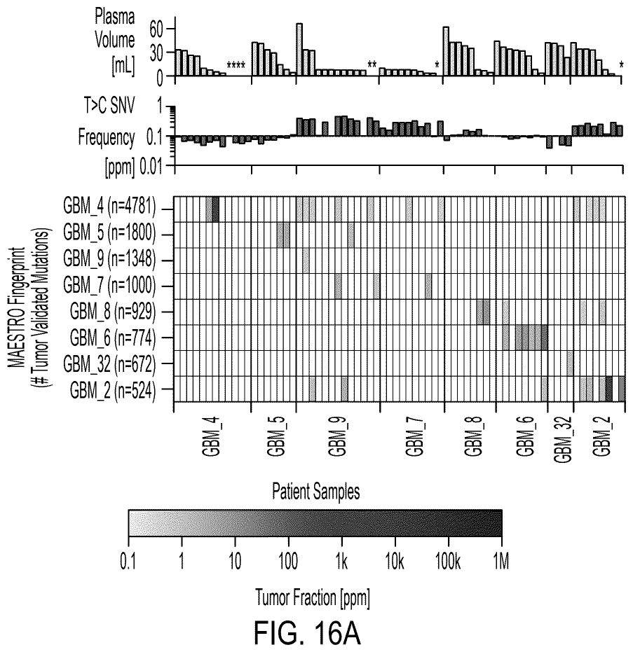

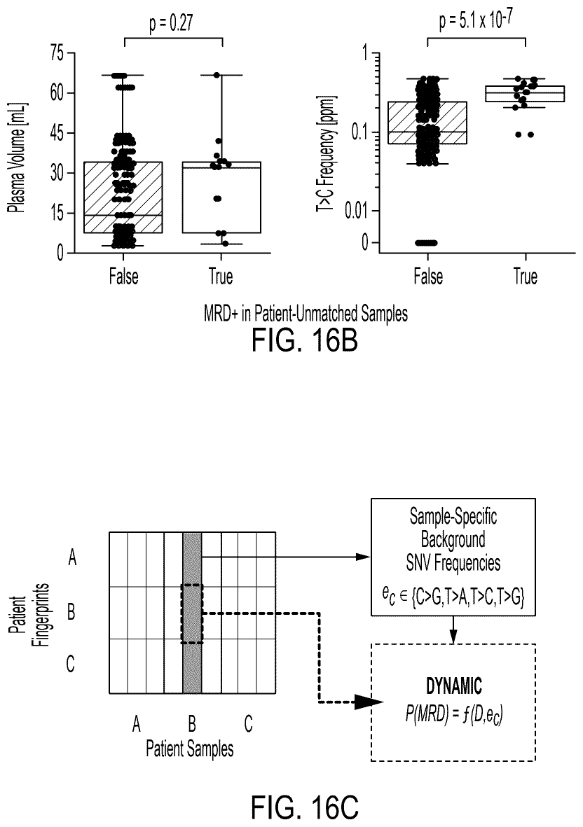

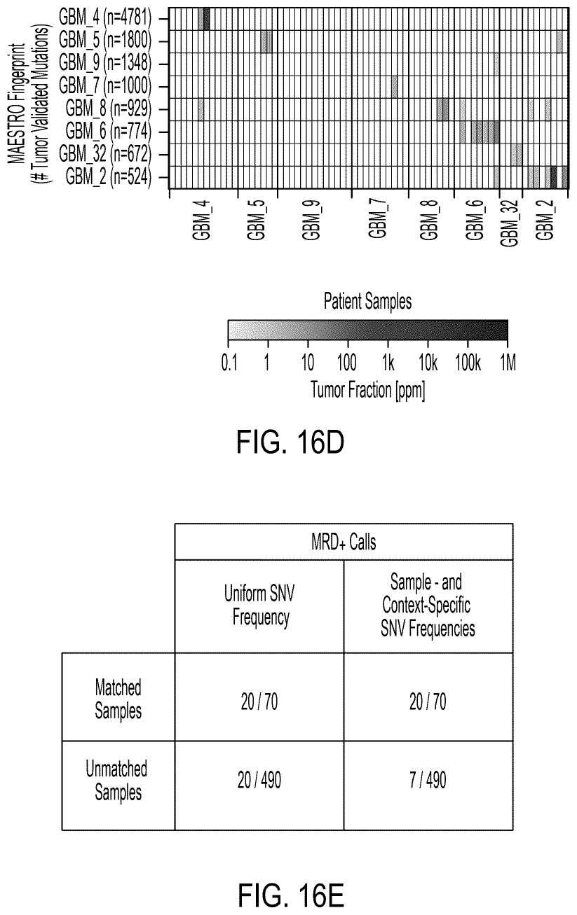

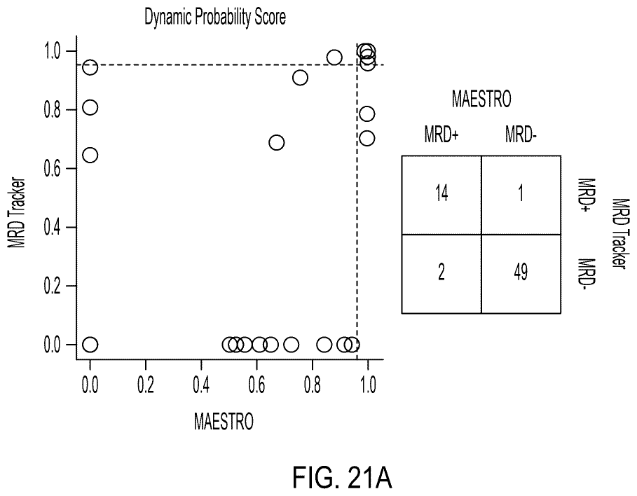

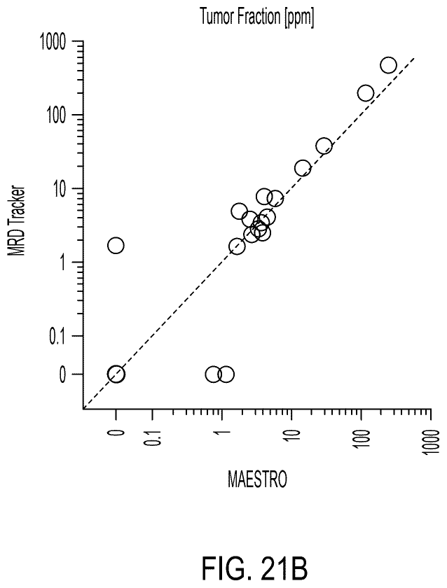

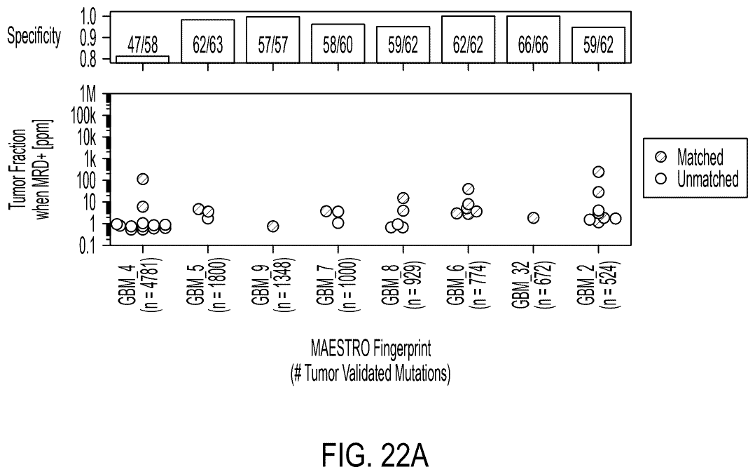

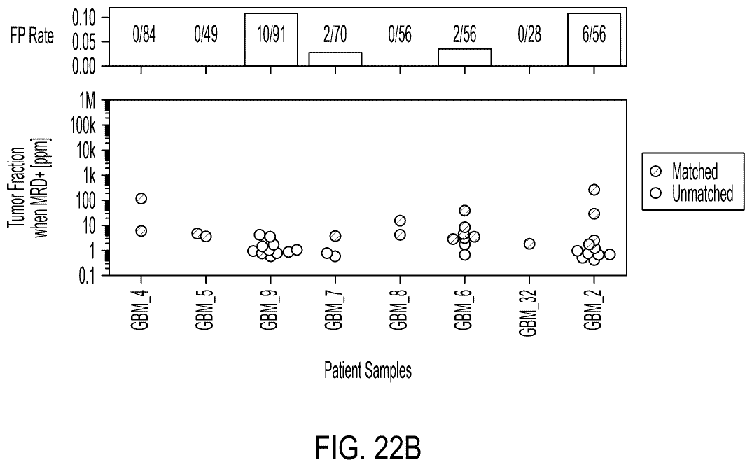

To detect MRD with MAESTRO-Pool, the validated dynamic MRD caller, described herein in Example 1, was first applied, which uses sample-specific attributes (such as observed tumor fraction, total duplexes evaluated, and fingerprint size) to estimate the probability that the observed MRD signal was true tumor signal as opposed to artifact from the background SNV frequency observed in cfDNA. As in Example 1, a uniform background SNV frequency of 0.1 ppm was assumed for all samples. In patient-matched samples, this resulted in MRD-positive calls for 20 of 70 samples with a median tumor fraction of 3.9 ppm (range 0.8-254.1 ppm; FIG. 16A and FIG. 16E). Of the 66 samples that were also tested with MRD Tracker, 63 samples had concordant MRD calls and tumor fractions (FIGS. 21A-21B). In patient-unmatched samples, however, 20 of 490 samples (4.1%) were falsely called MRD-positive with a median tumor fraction of 0.9 ppm (range 0.5-4.0 ppm; FIG. 16A, FIG. 16E, and FIGS. 22A-22B). Notably, the MAESTRO fingerprint for patient GBM_4, which was the largest fingerprint (n=4,781) due to Lynch syndrome-associated hypermutation, had the lowest specificity (specificity=0.81; FIG. 22A) and comprised a majority of the false positive MRD calls (n=11/20). Because the larger plasma volume collection was the major difference between Example 1 and Example 2, whether the false positives were due to larger plasma volumes was first investigated. Of the 20 MRD-positive unmatched samples, their plasma volumes were not significantly larger than their MRD-negative counterparts (Mann-Whitney U p=0.27; FIG. 16B), demonstrating that the false positives were not due to larger plasma volumes.

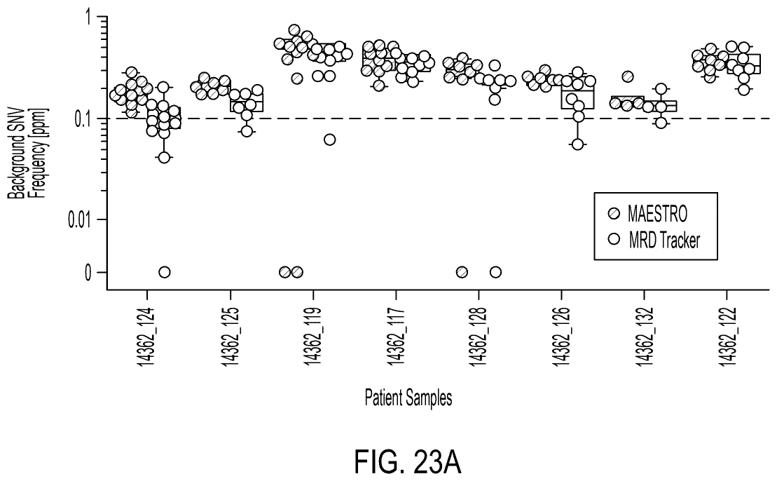

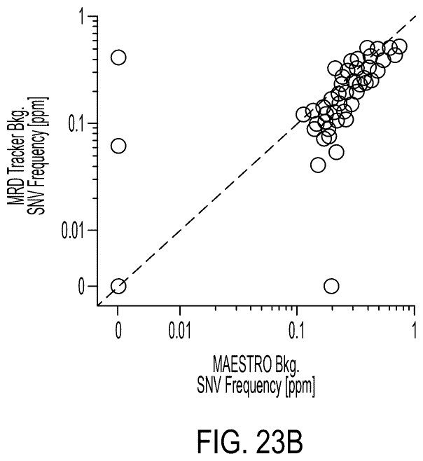

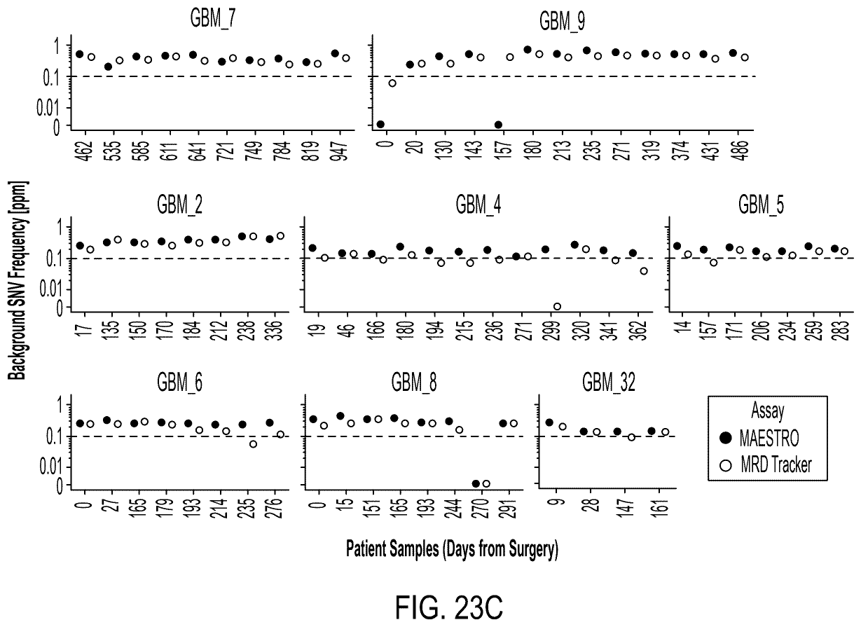

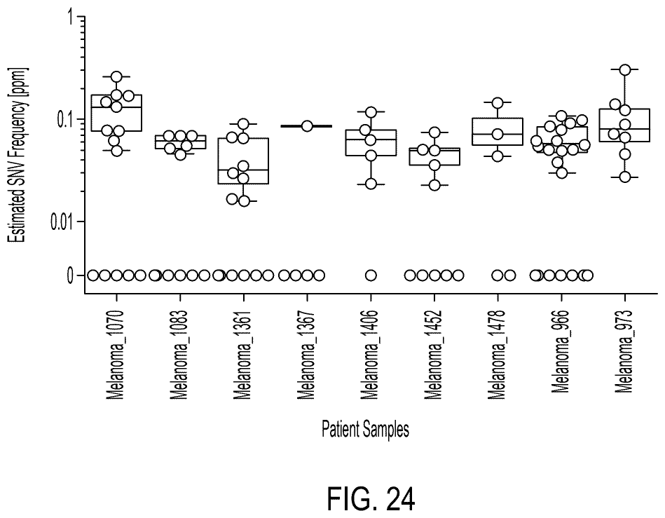

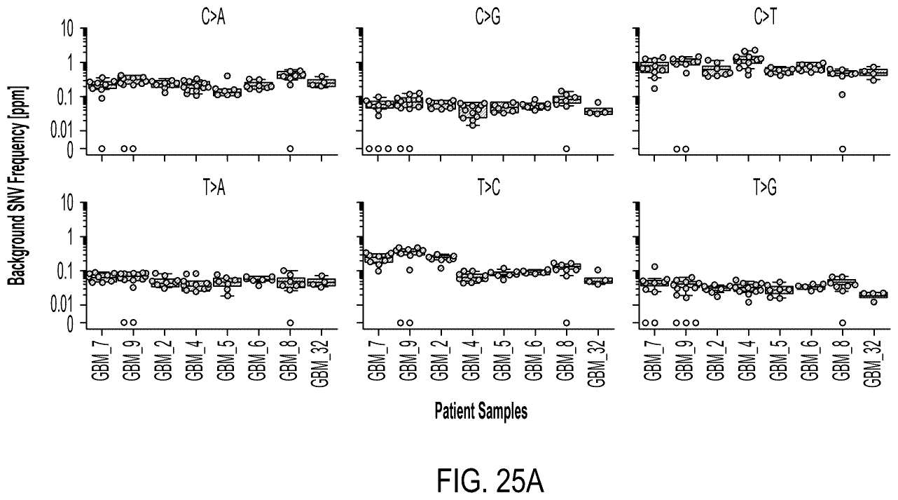

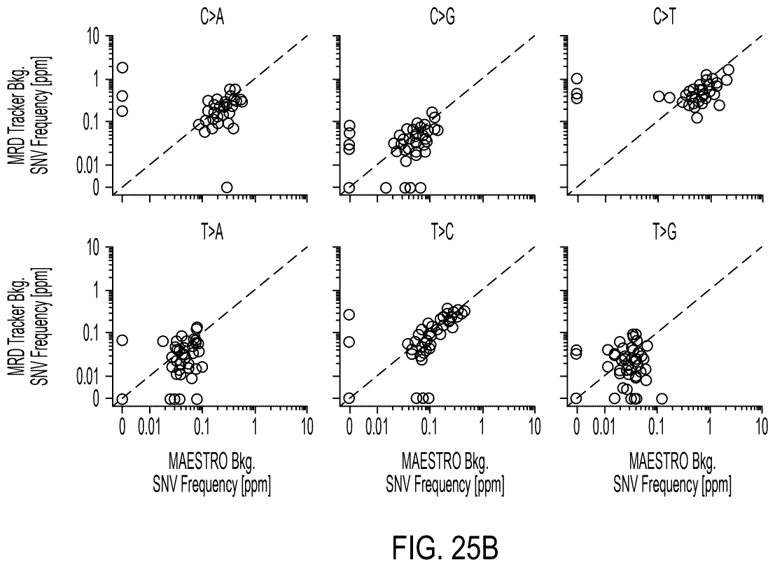

It was hypothesized that the cfDNA samples had higher background SNV frequencies than the assumption of 0.1 ppm, causing the dynamic caller to overestimate the probability of MRD. To investigate this, the sample-specific SNV frequencies from the MAESTRO-Pool data were quantified (FIGS. 23A-23C; Methods) and it was confirmed that multiple samples had background SNV frequencies higher than the 0.1 ppm assumption (FIG. 23A). This finding was rather unexpected, as 87 of 98 samples (89%) from Example 1 showed background SNV frequencies of consistently ~0.1 ppm or lower (FIG. 24). Notably, the patients with the highest median SNV frequencies (GBM_9=0.5 ppm, GBM_7=0.4 ppm, GBM_2=0.4 ppm) accounted for 18 of the 20 false positive samples (FIG. 22B). To understand the source of noise, the SNV frequencies were deconstructed by their mutation context (i.e., C>G, T>A, T>C, T>G). This further revealed that the elevated SNV frequencies for patients GBM 2, GBM 7, and GBM 9 were particularly due to their higher T>C frequencies (FIG. 16A and FIGS. 25A-25B), with MRD-positive unmatched samples exhibiting significantly higher T>C frequencies than the MRD-negative unmatched samples (Mann-Whitney U p=5.1×10⁻⁷; FIG. 16B). As orthogonal validation, the background SNV frequencies were estimated from MRD Tracker applied to the same sequencing libraries. The estimated SNV frequencies from MRD Tracker were highly concordant with those from MAESTRO-Pool, corroborating the elevated T>C frequencies in many samples (FIGS. 23A-23C and FIG. 25B).

Of note, examination of their clinical features revealed that these were the only glioblastomas in the cohort that had MGMT promoter methylation and received temozolomide alkylating chemotherapy. In MGMT promoter-methylated glioblastomas, temozolomide can induce an acquired mismatch repair deficiency. Given that mismatch repair deficiency is associated with T>C mutational patterns, these findings may suggest prior temozolomide as a source of the observed T>C error rate, although further investigation is warranted. Although temozolomide induces a C>T mutation signature, the estimated background C>T frequencies from patients GBM_7, GBM_9, and GBM_2 were not disproportionately elevated (FIGS. 25A-25B). However, C>T mutations intrinsically have higher background SNV frequencies than other mutation contexts (~1 ppm vs 0.1 ppm), so differences may be harder to resolve.

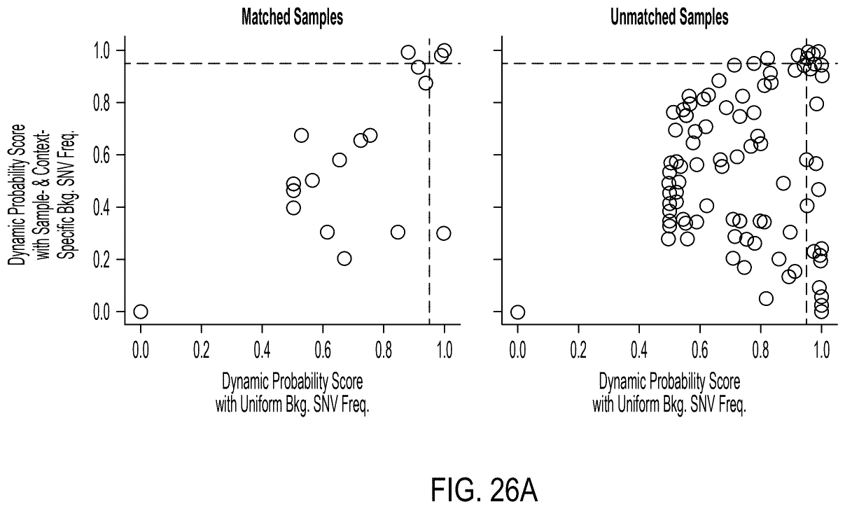

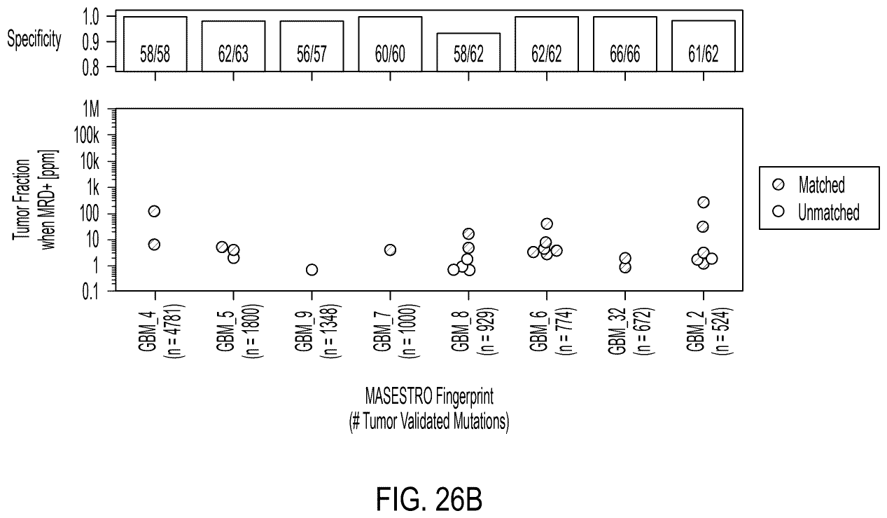

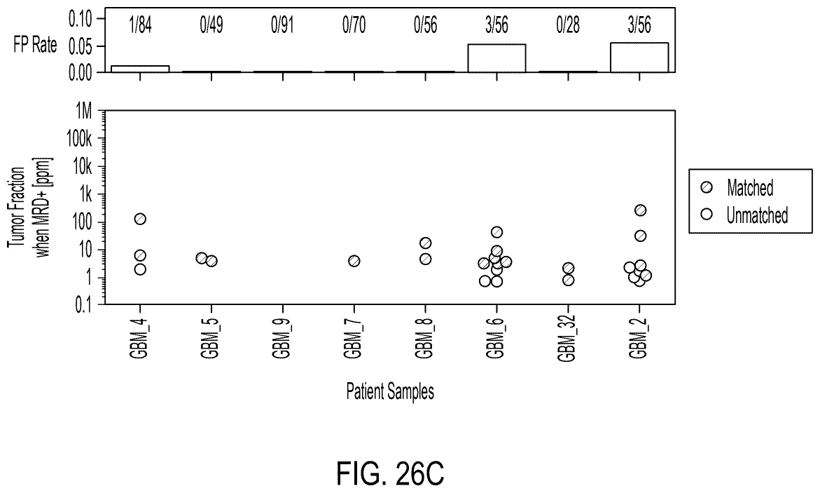

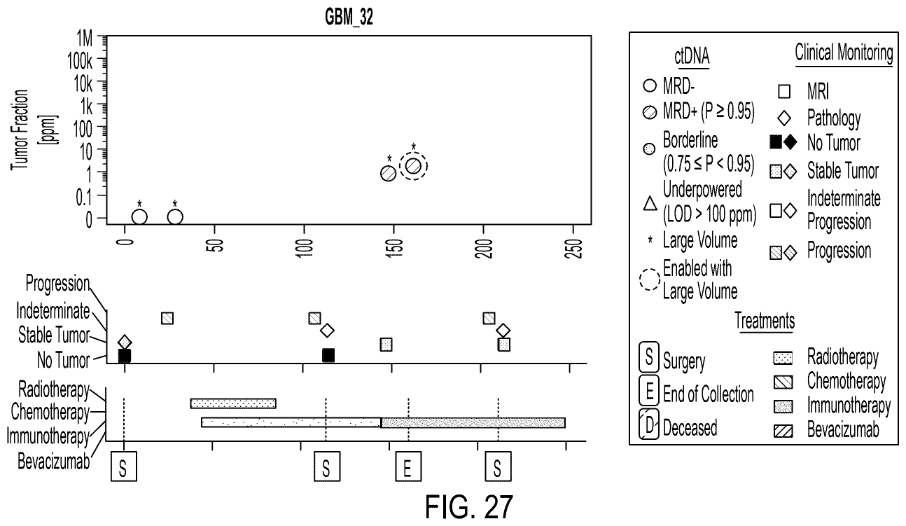

Based on the above observations of SNV-specific background, a new MRD calling strategy was developed by using the sample- and context-specific SNV frequencies to tune the assumed SNV frequency of the dynamic caller (FIG. 16C). More specifically, the dynamic caller was adapted to allow each mutation context to be tuned and weighed independently (Methods). This approach indeed suppressed the majority of false positive MRD calls among patient-unmatched samples (16 of 20, FIGS. 16D-16E) and improved the specificity of most MAESTRO panels to near 100% (median specificity=99%, range 94-100%; FIGS. 26A-26C). Notably, there were 3 false positives introduced with tuning, due to their measured SNV frequencies being lower than 0.1 ppm, which caused the dynamic caller to estimate dynamic probability scores higher than 0.95. Furthermore, tuning the dynamic caller had minimal impact on the MRD calls of patient-matched samples. Of the 20 MRD-positive calls without tuning, 19 remained after tuning, while one additional MRD-positive call was also made during tuning (FIGS. 16D-16E and FIGS. 26A-26C). Notably, the sample that became MRD-positive with tuning (day 147 from GBM_32; FIG. 27) precedes another MRD-positive sample 14 days later with similar tumor fraction, further supporting the presence of MRD and highlighting the impact of sample- and context-specific tuning.

### Larger Plasma Volume Collection Improves the Sensitivity of MRD Detection and Accuracy of Tumor Fraction Estimation

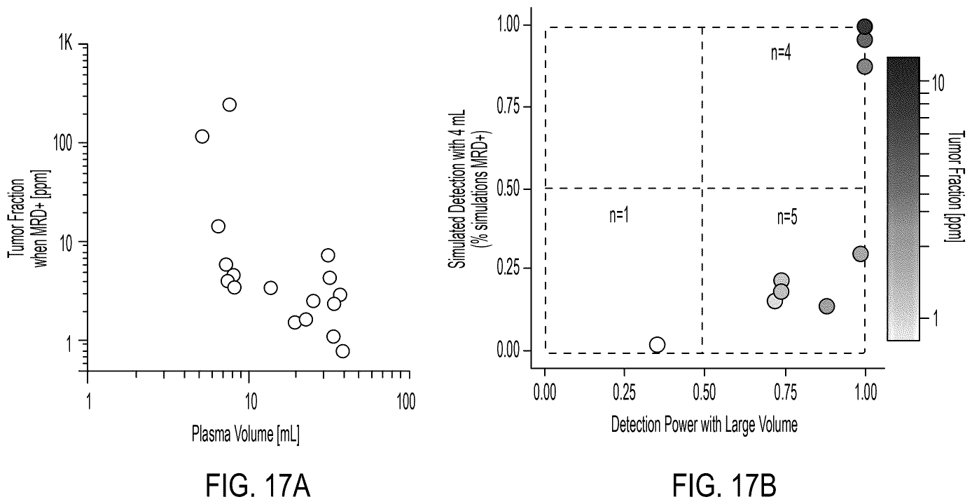

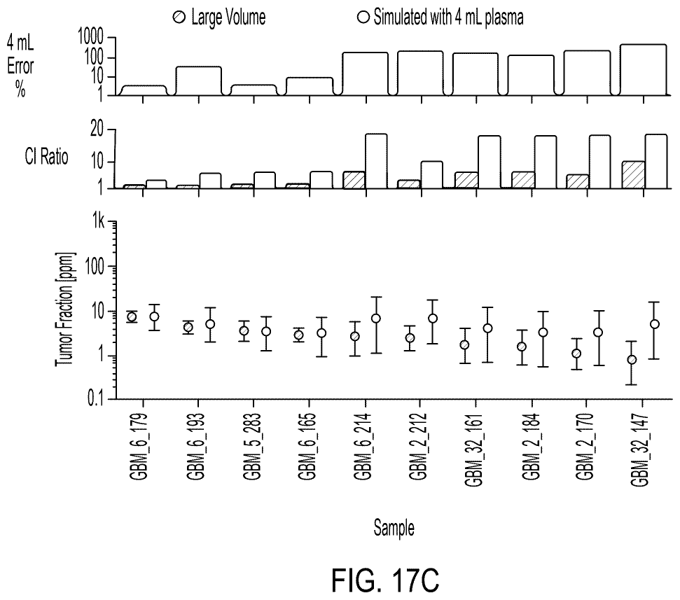

Among all MRD-positive samples, larger plasma volume collection detected lower tumor fractions (median tumor fraction=2.7 ppm, range 0.8-7.6 ppm; Mann-Whitney U p=3.6×10⁻³) than typical volume collection (median tumor fraction=5.9 ppm, range 3.5-254.1 ppm; FIG. 17A). To further assess the impact of larger plasma volume collection on MRD detection, data were simulated for single-blood-tube equivalents (i.e., 4 mL of plasma) by down-sampling the DNA duplexes recovered from MRD-positive samples with >10 mL plasma. This process was simulated 50 times for each sample, allowing for the estimation of the likelihood of retaining the MRD-positive status. Of the 10 MRD-positive samples with >10 mL plasma, n=3, n=1, and n=6 samples were called MRD-positive in ≥95%, 50%-95%, and <50% of simulations with single-blood-tube equivalents, respectively (FIG. 17B). For the 6 samples that were called MRD-positive in <50% of simulations, 5 samples had ≥50% power to detect the observed tumor fraction with the full plasma volume, doubling ctDNA-positivity and referred to as ‘enabled with large volume collection’ (FIG. 17B). These tended to have larger plasma volumes collected (median 24.2 mL, range 7.5-34.0 mL), greater cfDNA yields (median 118.0 ng, range 33.8-243.9 ng), and lower tumor fractions (median 2.2 ppm, range 1.2-4.1 ppm), suggesting that higher cfDNA yield (i.e., by larger volume collection) improves the ability for detecting low ppm levels of ctDNA. The impact of plasma volume on tumor fraction estimation was also examined. Expectedly, differences were found in tumor fraction estimates between large volume samples and their single-blood-tube equivalents (median error for single-blood-tube equivalents=114.5%, range 2.0-521.3%; FIG. 17C) with the largest differences observed for the lowest tumor fractions. Furthermore, the simulated single-blood-tube equivalents exhibited larger confidence interval ratios (median 3.0-fold increase in confidence interval ratio, range 1.8-3.8; FIG. 17C).

Association of MRD Detection with Clinical Responses in Glioblastoma Patients

Having ascertained the performance characteristics of MAESTRO-Pool MRD detection, it was next evaluated as a secondary analysis whether MAESTRO-Pool could be helpful in the clinical context of distinguishing radiographic pseudo-progression from bona fide tumor progression. Comparing the performance of the ctDNA-based assay to that of standard-of-care MRI and histopathology for glioblastoma was the focus (Table 1).

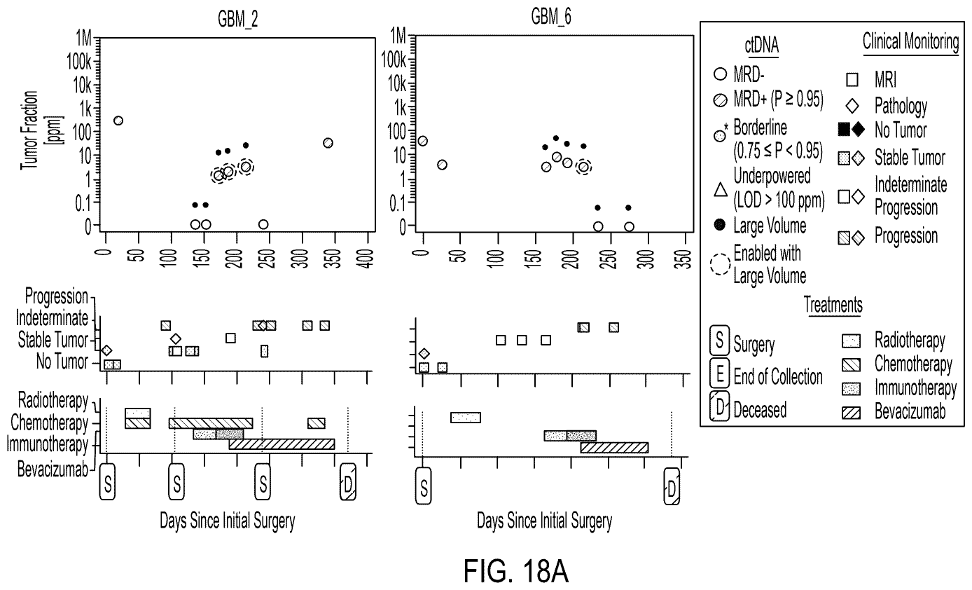

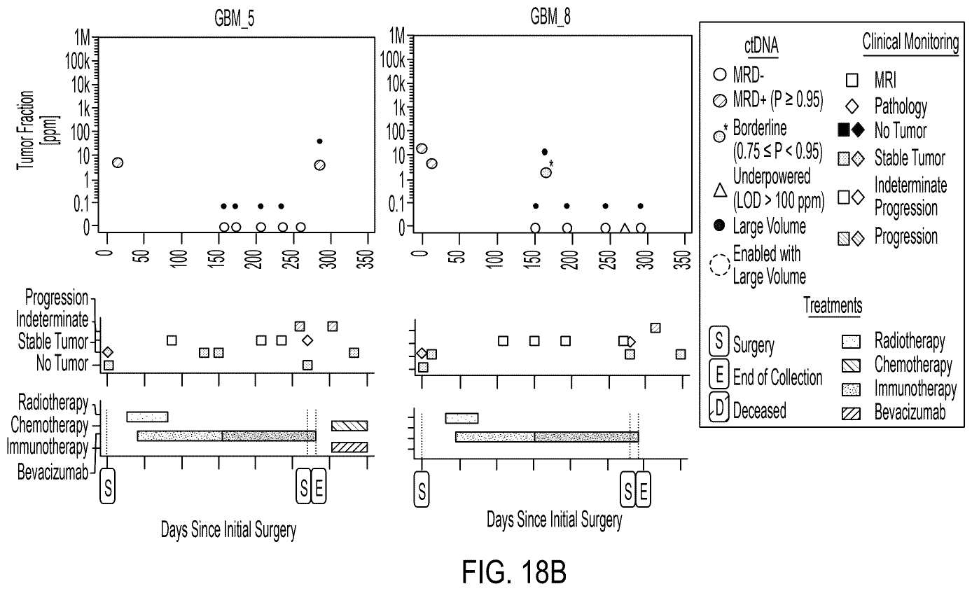

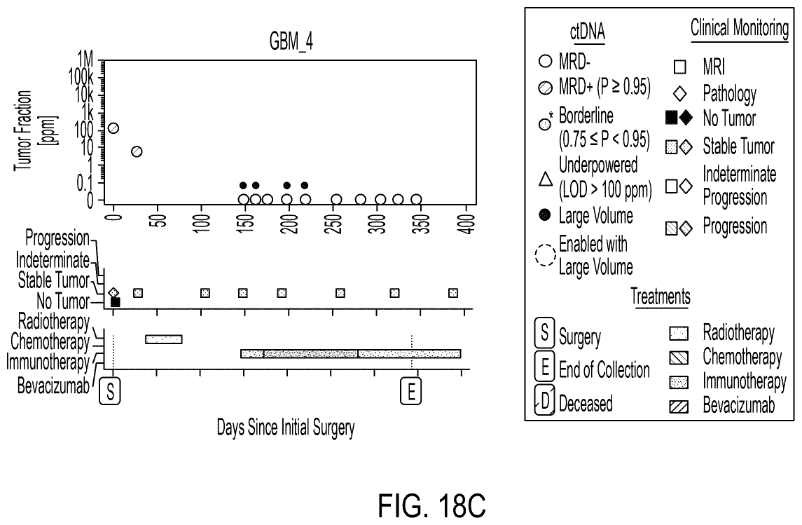

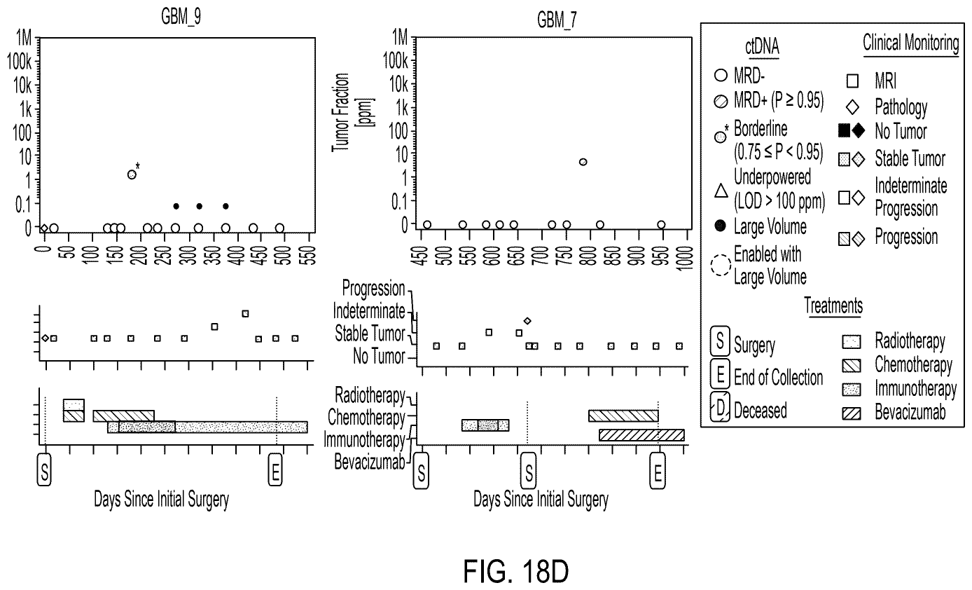

Overall, MRD was detected in at least 1 timepoint for 7 of 8 (87.5%) glioblastoma patients, with a borderline MRD call (0.75≤P≤0.95) detected in the eighth patient (FIGS. 18A-18D). Among the 7 patients with plasma collected within 3 weeks of initial surgery, 5 (71.4%) had plasma MRD detected. From 7 of 8 patients, 3 patterns of association were discerned between ctDNA assay results, radiographic assessment, and pathologic assessment, as described below. The eighth patient (GBM_32) had infrequent MRIs, complicating the comparison of radiographic findings with ctDNA assay results (FIG. 27).

In ‘Pattern 1,’ MRD positivity preceded histologic tumor progression, whereas the concurrent radiographic findings were indeterminate (i.e., true progression vs. pseudo-progression; GBM_2, GBM_6; FIG. 18A). In GBM_2, MRI (day 191) during immunotherapy and chemotherapy was reported as indeterminate progression (i.e., pseudo-progression vs. tumor progression); however, the three proximal plasma time-points (days 170, 184, 212) were all MRD-positive (tumor fractions 1.2-2.6 ppm; FIG. 18A-left). These findings were consistent with the subsequent MRI (day 233) and surgical resection (day 241), which both confirmed tumor progression, suggesting that MRD positivity preceded the radiographic confirmation of tumor progression by 63 days. Based on the simulations using 4 mL plasma volume, detection of all three MRD-positive timepoints was only made possible by the larger plasma volumes. Conversely, a typical volume plasma sample was collected on day 238, three days prior to the surgery, but no MRD was detectable, further supporting the hypothesis that larger volumes can circumvent false negatives. During surgery, only debulking of the tumor was attained and subsequent imaging displayed persistent tumor progression (days 254-336), which coincided with MRD detection (day 336; final plasma timepoint before the patient passed away). Similarly, GBM_6 was reported to have indeterminate progression on post-radiotherapy imaging (days 104-165), followed by tumor progression reported on MRIs between days 214-256; however, MAESTRO-Pool detected MRD during days 165-214 (tumor fractions 2.7-7.6 ppm; FIG. 18A-right), preceding radiographic determination of tumor progression by 49 days. As in GBM_2, MRD detection at an earlier timepoint (day 165) was enabled by larger plasma volume. MRD was not detected for GBM 6 in the subsequent timepoints (days 235, 276), although an intervening MRI reported persistent tumor progression. These findings suggest that large-volume MAESTRO-Pool MRD testing may have a role (e.g., in conjunction with standard contrast-enhanced MRI) in the sensitive and earlier detection of true glioblastoma progression.

In ‘Pattern 2,’ MRD negativity based on the analysis of large plasma volumes was associated with a lack of tumor progression, despite multiple concurrent post-radiotherapy MRIs that were reported as indeterminate progression (GBM_5, GBM_8, FIG. 18B). Both patients subsequently underwent surgical resection for the indeterminate progression, with pathology reporting <50% viable tumor and no evidence of histologic progression, thus confirming that the MRI findings were most consistent with pseudo-progression. Furthermore, for GBM_5, an MRI 11 days before the pathology finding of pseudo-progression was reported as tumor progression (day 259), whereas the corresponding plasma timepoint (day 259) was MRD-negative. Altogether, these results suggest that large-volume MRD testing may also have a role (e.g., in conjunction with standard contrast-enhanced MRI) in the specific diagnosis of pseudo-progression, thereby potentially helping to spare patients from repeat surgery if they are symptomatically stable. A similar pattern was observed for GBM 4, in which persistent negative MRD followed surgery, concordant with a lack of evidence of tumor progression on imaging (FIG. 18C). However, this patient was uniquely characterized by germline mismatch repair deficiency and experienced durable response to immune checkpoint blockade, with a small nodular enhancement around the surgical cavity that slowly resolved over time.

Finally, in ‘Pattern 3,’ there was radiographic and/or pathologic evidence of tumor progression; however, MRD was not detected in the preceding timepoints (GBM_7, GBM_9; FIG. 18D). The negative timepoints may be in part due to their need for greater T>C noise tuning, potentially due to the effects of MGMT promoter methylation and/or temozolomide in these tumors. Additionally, MAESTRO-Pool was underpowered to detect MRD in the perioperative timepoint of GBM_9 with LOD95>100 ppm due to typical plasma volume with low cfDNA yield (~3 ng), and none of GBM_7's plasma samples were of large volumes.

### Discussion

Liquid biopsy holds promise for improving cancer detection and monitoring but remains constrained by the scarcity of ctDNA in the bloodstream. The transient attenuation of cfDNA clearance has been proposed as one solution to improve the recovery of ctDNA and enhance liquid biopsy performance. Indeed, the first intravenous priming agents that temporarily reduced the clearance of cfDNA and recovered up to 60-fold more ctDNA in mice were recently described. Priming agents could therefore enable greater ctDNA recovery without the need to draw excessive volumes of blood. However, priming agents are not yet available for testing in humans, and there are little clinical data on the assay performance characteristics associated with higher yields of cfDNA due to the limited blood volumes typically drawn in routine clinical practice (i.e., one or two 10 mL tubes). Herein, up to ~15-fold more blood volume than a single tube blood draw was analyzed, and some of the first insights into the impact of higher cfDNA yields are provided. MAESTRO-Pool was also optimized and applied to harness the higher inputs of cfDNA and safeguard against false detection due to sample-intrinsic variations.

The MAESTRO technology targets thousands of genome-wide single nucleotide variants (SNVs) per patient using probes that preferentially hybridize to mutant DNA molecules and enrich their abundance after hybrid capture, and therefore reduces Duplex Sequencing costs by up to a hundred-fold. Duplex sequencing, in which both strands of DNA fragments are barcoded and only mutations represented in both strands are considered, substantially improves sequencing accuracy. Consequently, MAESTRO is well-suited for tracking large numbers of mutations and harnessing all available cfDNA. To further improve the specificity of MAESTRO, a new dynamic MRD caller was developed that incorporates sample-specific background SNV frequencies. This helped to safeguard against false detection in “edge cases” with atypical background SNV frequencies, as it was found for plasma samples from three MGMT promoter methylated patients treated with temozolomide, for whom elevated T>C background SNV frequencies were posited, may be therapy-related. Additionally, having MAESTRO-Pool data from both patient-matched and unmatched fingerprints allowed for sampling of sufficient cfDNA duplexes and thus, accurately quantifying low level (<1 ppm) sample-specific background SNV frequencies.

IDH-wildtype glioblastoma was selected as an ideal study context given its common classification as a ‘low-shedding’ tumor type and its unmet need for minimally invasive diagnostics that can longitudinally and accurately monitor disease status. The distinct biology of the blood-brain barrier and interstitial fluid drainage of the brain has been postulated to limit the presence of glioblastoma ctDNA in the plasma. This theory is supported by high rates of glioblastoma ctDNA detected in studies of cerebrospinal fluid (CSF) but not in several studies of plasma. For example, in one study, the CSF of all 9 evaluated glioblastoma patients yielded detectable ctDNA by mutation-specific digital droplet PCR. In another instance, among 11 IDH-wildtype glioblastomas with positive CSF ctDNA, only 1 (11%) also had ctDNA detected in the plasma. Similarly, early pan-cancer studies that used typical 1-2 blood tube collections detected plasma ctDNA in less than 30% of glioblastoma patients. Finally, in another study of 20 high-grade glioma patients, although 55% had mutations detected in the pre-operative plasma using the non-personalized Guardant360 NGS-based ctDNA assay, none of the mutations were shared with the corresponding tumor tissue sequencing, suggesting that many may have represented false positives (e.g., from clonal hematopoiesis).

By contrast herein, tumor-specific ctDNA was detected in 71% (5/7) of glioblastoma patients within 3 weeks of their initial surgery and 88% (7/8) of glioblastoma patients overall, including at multiple timepoints for 75% (6/8) of patients. Taken together, these findings demonstrate that recovering more cfDNA than typically collected in routine clinical practice can help improve ctDNA detection. It was also found that accounting for variations in background SNV frequencies among samples reduced the number and fraction of patient-unmatched tests that were falsely positive without compromising sensitivity. The importance of such improved performance characteristics is emphasized, as efforts in the liquid biopsy field are increasingly evaluating higher cfDNA yields per sample and are seeking to more accurately detect ctDNA at parts-per-million levels.

As a secondary analysis, the utility of MAESTRO-Pool was evaluated in the assessment of bona fide tumor progression vs. pseudo-progression, which are challenging to distinguish using contemporary MRI techniques, in 8 glioblastoma patients. In the setting of indeterminate progression on contrast-enhanced MRI, the data suggest that MAESTRO-Pool with large plasma volumes may be able to improve the sensitivity of true progression detection (Pattern 1) and the specificity of pseudo-progression determination (Pattern 2). Earlier detection of tumor progression enables glioblastoma patients who are no longer responding to their current therapy and may benefit from enrollment on a new clinical trial to be identified, whereas the accurate diagnosis of pseudo-progression can help spare glioblastoma patients from unnecessary surgery if they are symptomatically stable.

Altogether, these findings show that increasing cfDNA recovery improves liquid biopsy testing in a low ctDNA shedding cancer such as glioblastoma. Efforts to recover more cfDNA from the body may help to make liquid biopsies more informative for more patients with a spectrum of cancer types.

TABLE 1
Radiographic and Pathologic Response Assessments.

| | Representative neuro- | |
|---|---|---|
| | radiologist/neuro-oncologist | Representative neuro- |
| Response assessments | terminology | pathologist terminology |
| Tumor | Likely progressive; Recurrence; | Viable tumor > 50% |
| Progression/Recurrence | Increase in size of ≥25% | |
| Pseudo-progression vs. | Pseudo-progression, Treatment effect, | Viable tumor 0 < % < 50; |
| Tumor progression | or Radio-necrosis vs. progressive | Evidence of histological |
| | disease (e.g., cannot rule out | progression: not present; |
| | glioma/progressive disease, | Treatment effect |
| | underlying tumor). | |
| Tumor (Stable/No definitive | Stable disease; Interval increase; | Glioblastoma; Glioma |
| evidence of | Interval decrease; No evidence of | |
| progression/Treatment | progression/recurrence; Treatment | |
| response) | response; Subtotal resection | |
| No definitive tumor | Consistent with gross total resection; | No evidence of tumor |
| | No residual tumor | |

### Additional Methods for Example 2

### MAESTRO-Pool and MRD Tracker Assays

In brief, for MAESTRO-Pool, hybrid capture was performed using xGen™ Hybridization and Wash Kit with xGen™ Universal Blockers (IDT). Each hybrid capture contained a maximum of 12 samples, with a library mass equivalent to 50 times DNA mass into library construction for each sample and used 4 μmol of the MAESTRO-Pool panel. The hybridization program began at 95 °C for 30 seconds, followed by a stepwise decrease in temperature from 65 °C to 50 °C, dropping 1 °C every 48 minutes. Finally, the plate was held at 50 °C for at least four hours. Heated wash steps were performed at 50 °C. After the first round of hybrid capture, 16 cycles of PCR were applied. The product was subject to a second round of hybrid capture using 2 μmol of the MAESTRO-Pool panel. This was followed by another 16 cycles of PCR. Final captured product was quantified and pooled for sequencing on an Illumina® NovaSeq™ S4 (151 bp paired-end reads) with a target raw depth of 10,000× per site per 20 ng DNA mass into library construction.

MRD Tracker hybrid capture was performed with some notable differences from MAESTRO-Pool above, including: (1) A library mass equivalent to 25 times DNA mass into library construction was used for hybrid capture; (2) The hybridization program began at 95 °C for 30 seconds, followed by staying at 65 °C for at least four hours; (3) Heated wash steps were performed at 65 °C. Final captured product was sequenced with a target raw depth of 10,000× per site per 20 ng DNA mass into library construction.

### Estimating Sample-Specific SNV Frequencies

To quantify the sample-specific SNV frequencies, the regions of captured cfDNA molecules flanking the probe binding region were leveraged. It was reasoned that captured and sequenced bases overlapping the probe would not represent the sample-specific SNV frequencies due to MAESTRO's mutation enrichment. However, MAESTRO probes are small (~30 bp) relative to the typical size of cfDNA (~167 bp), so most bases in each captured molecule do not interact with the MAESTRO probe and thus, should be representative of the sample-specific background SNV frequencies. Therefore, the MAESTRO data were first restricted to +250 bp of the probed regions. Then, the same fragment-level and site-level filters used for MRD detection were applied to remove effects of known technical artifacts (see MAESTRO-Pool above). This yielded counts for the number of reference bases observed and the number of SNVs observed per sample, which were used to calculate sample- and context-specific background SNV frequencies.

Dynamic MRD Caller with Noise Tuning

The dynamic MRD caller, which weighs sample-specific attributes (such as observed tumor fraction, validated fingerprint size, and cfDNA mass) and quantifies the probability of the observed data being the result of true tumor signal rather than false, spontaneous errors, is described in Example 1. From analyzing close to 100 negative control samples in previous studies, the background SNV frequency has consistently been observed to be ≤0.1 ppm. Therefore, this was assumed as the dynamic caller's default value for the background SNV frequency and this approach was applied to all MAESTRO-Pool and MRD Tracker samples in this Example 2. Additionally, a dynamic probability threshold of P≥0.95 was used to call MRD-positive samples as described in Example 1.

Here, the dynamic MRD caller was adapted to enable context-specific noise tuning. This was accomplished by assuming independence between mutation contexts as follows:

$$P ( {M}^{+} \vert D ) = \frac{P ( D \vert {M}^{+} ) P ( {M}^{+} )}{P ( D \vert {M}^{+} ) P ( {M}^{+} ) + P ( D \vert {M}^{-} ) P ( {M}^{-} )} \qquad Equation ( 4 )$$

$$D : Observed MAESTRO data$$

$${M}^{+} : MRD positive status$$

$${M}^{-} : MRD negative status$$

$$P ( D \vert {M}^{+} ) = {\prod }_{c} Bin ( ALT {duplexes}_{c} , total {duplexes}_{c} , \frac{{TF}_{x}}{2} ) \qquad Equation ( 5 )$$

$$for c ∈ \{ C > G , T > A , T > C , T > G \}$$

$$P ( D \vert {M}^{-} ) = {\prod }_{c} BetaBinom ( ALT {duplexes}_{c} , total {duplexes}_{c} , {α}_{c} , {β}_{c} ) \qquad Equation ( 6 )$$

$$for c ∈ \{ C > G , T > A , T > C , T > G \}$$

$${α}_{c} = {α}_{i} + [ SNVs {Observed}_{c} from SNV freq estimation ] \qquad Equation ( 7 )$$

$$where {α}_{i} = ( \frac{1 - {μ}_{i}}{{σ}_{i}^{2}} - \frac{1}{{μ}_{i}} ) {μ}_{i}^{2}$$

$$and {μ}_{i} = 0.05 ppm , {σ}_{i} = 0.05 ppm$$

$${β}_{c} = {β}_{i} + [ Bases {Sequenced}_{c} from SNV freq estimation ] - [ SNVs {Observed}_{c} from SNV freq estimation ] \qquad Equation ( 8 )$$

$$where {β}_{i} = {α}_{i} ( \frac{1}{{μ}_{i}} - 1 )$$

The two major changes occur in the likelihood functions where they become the product of each context-specific likelihood (Equations 5 and 6). Furthermore, the likelihood function for an MRD-negative status was changed to a beta binomial model, which allowed (1) a prior to be initialized for each context's background SNV frequency and (2) MAESTRO's measured sample-specific SNV frequency (see Estimating Sample-Specific SNV Frequencies) to tune each context's background SNV frequency. For (1), it was reasoned that establishing a prior would be useful since not every MAESTRO sample is powered to measure context-specific SNV frequencies down to 0.01 ppm. To account for this, a prior of Beta (mean=0.05 ppm, std=0.05 ppm) was initialized for each mutation context, translating to a conservative overall SNV frequency of 2 ppm (i.e., 0.5 ppm_{C>G}+0.5 ppm_{T>A}+0.5 ppm_{T>C}+0.5 ppm_{T>G}). For (2), MAESTRO's measured sample-specific SNV frequency, modeled as Binomial (#SNVs observed, #bases observed), could be combined with the prior to calculate the posterior, representing the tuned sample-specific SNV frequency for each mutation context. In summary, these changes enabled the dynamic caller to perform sample- and context-specific noise tuning when the background noise is known. It was used in conjunction with MAESTRO's measured SNV frequencies and applied to all MAESTRO-Pool samples.

### Example Environment

FIG. 33 is an illustration of an environment 3300 in another example implementation that is operable to employ dynamic MRD classification as described herein. The environment 3300 includes an adaptation of the environment 3200 of FIG. 32 that enables each mutation context to be tuned and weighted independently. Therefore, for brevity, the following discussion will focus on differences of the environment 3300 from the environment 3200 of FIG. 32, and components previously introduced in FIG. 32 are numbered the same and function as previously described.

In the environment 3300, the specificity factors 3226 further include context-specific factors 3302. The context-specific factors 3302 are determined for a plurality of mutation contexts, such as C to G mutations, T to A mutations, T to C mutations, and T to G mutations. Moreover, in at least one implementation, the specificity factors 3226 further include a sample-specific background mutation frequency 3304 that may be used in addition to or as an alternative to the assumed background mutation frequency 3232. The sample-specific background mutation frequency 3304 is an empirically derived background mutation frequency, e.g., as derived from the sample. By way of example, the sample-specific background mutation frequency 3304 may represent a sample-specific error rate. As such, in at least one implementation, a context-specific analysis enabled by the environment 3300 is performed with the assumed background mutation frequency 3232, while in at least one other implementation, the context-specific analysis enabled by the environment 3300 is performed using the sample-specific background mutation frequency 3304.

For instance, the specificity factor determination algorithm 3224 is adapted to quantify an observed background mutation frequency 3306, which may be a frequency at which SNVs are observed in the sample (see the Estimating Sample-Specific SNV Frequencies section). The specificity factor determination algorithm 3224 may be further adapted to determine a number of observed background bases 3308. The number of observed background bases 3308, for instance, refers to the number of sequenced bases analyzed. Together, the observed background mutation frequency 3306 and the number of observed background bases 3308 may be used to determine the sample-specific background mutation frequency 3304. In at least one implementation, the observed background mutation frequency 3306 and the number of observed background bases 3308 are determined for respective mutation contexts of the plurality of mutation contexts.

By way of example, the observed background mutation frequency 3306 and the number of observed background bases 3308 refer to the output from some implementations of the specificity factor determination algorithm 3224 that quantify the sample-specific background mutation frequency 3304. The sample-specific background mutation frequency 3304 is distinct from tumor signal and therefore should not analyze sites in the tumor fingerprint. For example, when analyzing MAESTRO data, the sample-specific background mutation frequency 3304 is quantified in regions surrounding and excluding the probed regions. Furthermore, the observed background mutation frequency 3306 refers to the count of mutated bases when assessing DNA duplexes in these regions, and the number of observed background bases 3308 refers to the total number of bases (wild type and mutated) when assessing DNA duplexes in these regions.

The context-specific factors 3302 are used by the tuning algorithm 3236 in addition to the total mutated duplexes 3228 and the total assumed duplexes 3230 to tune parameters of an MRD-positive likelihood model 3310 configured to output the first likelihood 3240 and an MRD-negative likelihood model 3312 configured to output the second likelihood 3244. As such, the MRD classification module 3234 may model the first likelihood 3240 and the second likelihood 3244 in different ways in the environment 3300 compared to the environment 3200 of FIG. 32.

The MRD-positive likelihood model 3310, for instance, determines a likelihood of the observed data given that the sample is MRD positive, such as according to Equation 5 given above. The MRD-positive likelihood model 3310 is different than the MRD-positive likelihood model 3238 of FIG. 32 in order to utilize the context-specific factors 3302. For example, the MRD-positive likelihood model 3310 may be a binomial probability calculation (e.g., a binomial model) that uses the total mutated duplexes 3228 and the total assumed duplexes 3230 for each mutation context c and may compute the first likelihood 3240 as the product of individual likelihoods for the respective mutation contexts of the plurality of mutation contexts. As such, the first likelihood 3240 corresponds to an overall likelihood taken over all of the mutation contexts.

The MRD-negative likelihood model 3312, for instance, determines a likelihood of the observed data given that the MRD status is negative, such as according to Equation 6 given above. The MRD-negative likelihood model 3312 may be a beta-binomial probability calculation (e.g., a beta-binomial model) that uses the total mutated duplexes 3228, the total assumed duplexes 3230, and parameters α_{c} and β_{c} for each mutation context c. The MRD-negative likelihood model 3312 may compute the second likelihood 3244 as the product of individual likelihoods for the respective mutation contexts of the plurality of mutation contexts. As such, the second likelihood 3244 corresponds to an overall likelihood taken over all of the mutation contexts.

In at least one implementation, the tuning algorithm 3236 may adjust a, according to Equation 7 above. For instance, αᵢ may be a baseline parameter calculated from Ali (e.g., the expected mean frequency of the SNVs in parts per million, which may be set to 0.05 ppm) and of (e.g., the expected standard deviation of the SNVs in parts per million, which may also be set to 0.05 ppm), which may be empirically derived constants reflecting expected variant frequencies and their variability. To determine ac, the observed background mutation frequency 3306 (e.g., the observed count of single nucleotide variants in the mutation context c, as determined from the SNV frequency estimation) is added to αᵢ (see Equation 7).

Similarly, in at least one implementation, the tuning algorithm 3236 may adjust β_{c} according to Equation 8 above. For instance, βᵢ may be a baseline expectation calculated based on αᵢ and μᵢ. To determine β_{c}, the number of observed background bases 3308 (e.g., the bases sequenced in the mutation context c) is added to βᵢ, and the observed background mutation frequency 3306 is subtracted (see Equation 8). As such, the observed background mutation frequency 3306 and the number of observed background bases 3308 may be used to tune the MRD-negative likelihood model 3312 for each mutation context.

The first likelihood 3240 and the second likelihood 3244 are used by the dynamic probability scoring algorithm 3246 to output the dynamic probability score 3248, such as described above with respect to FIG. 32. By way of example, the dynamic probability scoring algorithm 3246 may use a Bayesian probability calculation, such as Equation 4 provided above.

In at least one implementation, the dynamic probability scoring algorithm 3246 computes prior probabilities (also referred to as “priors”) using the initialized parameters (e.g., βᵢ and αᵢ), and then the sample-specific background mutation frequency 3304 (e.g., modeled as Binomial (observed background mutation frequency 3306, number of observed background bases 3308)) is combined with the prior probabilities to calculate the dynamic probability score 3248.

The dynamic probability score 3248 enables comparison of ctDNA signal between samples. For example, when using the dynamic model for MRD classification of MAESTRO-Pool data, the dynamic probability scores can be compared between patient-matched samples and patient-unmatched samples. One application is the identification of borderline positive samples, which are patient-matched samples that have a probability score below the threshold (e.g., P<0.95) but above most patient-unmatched samples.

In this way, the MRD classification module 3234 is adapted to perform sample- and context-specific noise tuning when the background noise is known.

### Example Procedure

This section describes an example procedure for performing dynamic MRD classification in one or more implementations. Aspects of the procedure may be implemented in hardware, firmware, or software, or a combination thereof. The procedure is shown as a set of blocks that specify operations performed by one or more devices and is not necessarily limited to the orders shown for performing the operations by the respective blocks. In at least some implementations, the procedure is performed by a suitably configured device, such as the sequencing data processor 3206 of FIGS. 32 and 33.

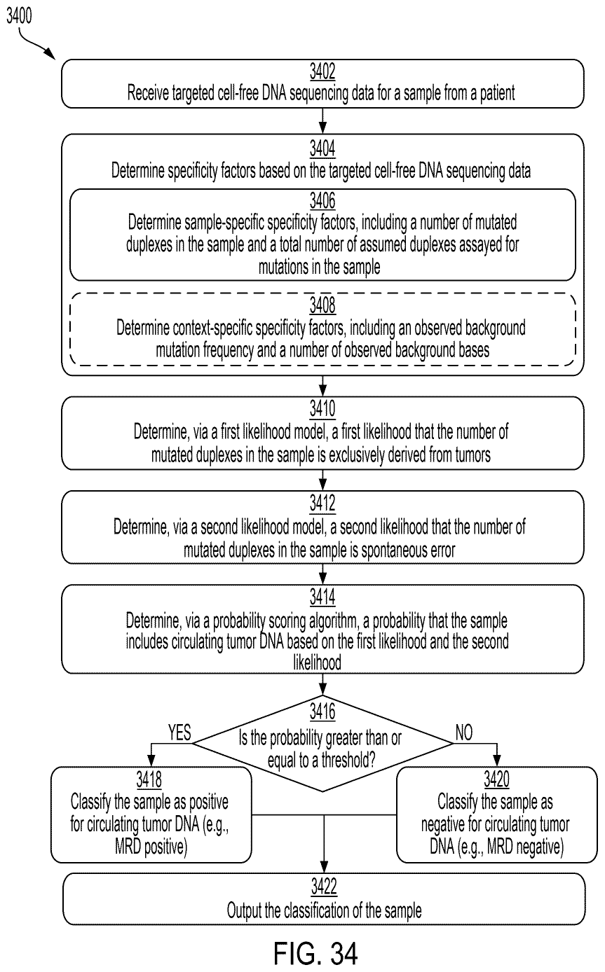

FIG. 34 depicts an example procedure 3400 for performing dynamic MRD classification.

Targeted cell-free DNA sequencing data for a sample from a patient is received (block 3402). For example, the targeted cell-free DNA sequencing data 3216 may be generated using MAESTRO or MAESTRO-Pool techniques as described herein. The targeted cell-free DNA sequencing data 3216 is enriched for DNA duplexes with mutation sites found in a tumor fingerprint associated with the patient. In some implementations, the targeted cell-free DNA sequencing data 3216 may be further enriched for DNA duplexes with additional mutation sites found in tumor fingerprints associated with other patients.

Specificity factors are determined based on the targeted cell-free DNA sequencing data (block 3404). This includes determining sample-specific specificity factors (block 3406), such as a number of mutated duplexes in the sample (e.g., total mutated duplexes 3228) and a total number of assumed duplexes assayed for mutations in the sample (e.g., total assumed duplexes 3230). Optionally, determining the specificity factors further includes determining context-specific specificity factors (block 3408), including an observed background mutation frequency and a number of observed background bases for respective mutation contexts of a plurality of mutation contexts.

A first likelihood that the number of mutated duplexes in the sample is exclusively derived from tumors is determined via a first likelihood model (block 3410). In at least one implementation, the first likelihood model may be the MRD-positive likelihood model 3238 of FIG. 32. Alternatively, the first likelihood model may be the MRD-positive likelihood model 3310 of FIG. 33. The first likelihood model may be a binomial model that uses the number of mutated duplexes and the total number of assumed duplexes assayed for the mutation sites in the sample along with an assumed background mutation frequency. In the example of FIG. 33, the first likelihood model may additionally or alternatively use a sample-specific background mutation frequency.

A second likelihood that the number of mutated duplexes in the sample is spontaneous error is determined via a second likelihood model (block 3412). In at least one implementation, the second likelihood model is the MRD-negative likelihood model 3242 of FIG. 32. In such implementations, the second likelihood model may be a binomial model that uses the number of mutated duplexes and the total number of assumed duplexes assayed for the mutation sites in the sample along with the assumed background mutation frequency. Alternatively, the second likelihood model may be the MRD-negative likelihood model 3312 of FIG. 33. In such implementations, the second likelihood model may be a beta-binomial model that uses the number of mutated duplexes and the total number of assumed duplexes assayed for the mutation sites in the sample as well as the observed background mutation frequency 3306 and the number of observed background bases 3308 for context-specific noise tuning.

A probability that the sample includes circulating tumor DNA is determined via a probability scoring algorithm based on the first likelihood and the second likelihood (block 3414). For example, the dynamic probability scoring algorithm 3246 may use a Bayesian probability calculation to output a dynamic probability score 3248.

It is determined if the probability is greater than or equal to a threshold (block 3416). In at least one implementation, the threshold is a pre-determined, adjustable value that is used to distinguish between samples that include circulating tumor DNA (e.g., MRD positive samples) from those that do not include circulating tumor DNA (e.g., MRD negative samples). As a non-limiting, illustrative example, the threshold is 0.95.

If the probability is greater than or equal to the threshold (YES branch of block 3416), the sample is classified as positive for circulating tumor DNA (e.g., MRD positive) (block 3418). If the probability is less than the threshold (NO branch of block 3416), the sample is classified as negative for circulating tumor DNA (e.g., MRD negative) (block 3420).

The classification of the sample (e.g., as MRD positive or MRD negative) is output (block 3422). For example, the MRD classification 3218 may be displayed via the display device 3252 of the client device 3204 or stored in memory for subsequent access.

In this way, the procedure 3400 generates an MRD classification for increased circulating tumor DNA detection sensitivity and a decreased probability of false detection, utilizing sample-specific and/or context-specific factors to adapt its confidence to the particular sample being evaluated.

### Example System and Device

FIG. 35 illustrates an example system generally at 3500 that includes an example computing device 3502 that is representative of one or more computing systems and/or devices that may implement the various techniques described herein. This is illustrated through inclusion of the sequencing data processor 3206. The computing device 3502 may be, for example, a server of a service provider, a device associated with a client (e.g., a client device), an on-chip system, and/or any other suitable computing device or computing system.

The example computing device 3502 as illustrated includes a processing system 3504, one or more computer-readable media 3506, and one or more I/O interfaces 3508 that are communicatively coupled, one to another. Although not shown, the computing device 3502 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 3504 is representative of functionality to perform one or more operations using hardware. Accordingly, the processing system 3504 is illustrated as including hardware elements 3510 that may be configured as processors, functional blocks, and so forth. This may include implementation in hardware as an application specific integrated circuit or other logic device formed using one or more semiconductors. The hardware elements 3510 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable storage media 3506 is illustrated as including memory/storage 3512. The memory/storage 3512 represents memory/storage capacity associated with one or more computer-readable media. The memory/storage 3512 may include volatile media (such as random-access memory (RAM)) and/or nonvolatile media (such as read only memory (ROM), Flash memory, optical disks, magnetic disks, and so forth). The memory/storage 3512 may include fixed media (e.g., RAM, ROM, a fixed hard drive, and so on) as well as removable media (e.g., flash memory, a removable hard drive, an optical disc, and so forth). The computer-readable media 3506 may be configured in a variety of other ways as further described below.

Input/output interface(s) 3508 are representative of functionality to allow a user to enter commands and information to computing device 3502 and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 3502 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hard-wired device that performs operations based on hard-wired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 3502. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 3502, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 3510 and computer-readable media 3506 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 3510. The computing device 3502 may be configured to implement particular instructions and/or functions corresponding to the software and/or hardware modules. Accordingly, implementation of a module that is executable by the computing device 3502 as software may be achieved at least partially in hardware, e.g., through use of computer-readable storage media and/or hardware elements 3510 of the processing system 3504. The instructions and/or functions may be executable/operable by one or more articles of manufacture (for example, one or more computing devices 3502 and/or processing systems 3504) to implement techniques, modules, and examples described herein.

The techniques described herein may be supported by various configurations of the computing device 3502 and are not limited to the specific examples of the techniques described herein. This functionality may also be implemented all or in part through use of a distributed system, such as over a “cloud” 3514 via a platform 3516 as described below.

The cloud 3514 includes and/or is representative of a platform 3516 for resources 3518, which are depicted including the sequencing data processor 3206. The platform 3516 abstracts underlying functionality of hardware (e.g., servers) and software resources of the cloud 3514. The resources 3518 may include applications and/or data that can be utilized while computer processing is executed on servers that are remote from the computing device 3502. Resources 3518 can also include services provided over the Internet and/or through a subscriber network, such as a cellular or Wi-Fi network.

The platform 3516 may abstract resources and functions to connect the computing device 3502 with other computing devices. The platform 3516 may also serve to abstract scaling of resources to provide a corresponding level of scale to encountered demand for the resources 3518 that are implemented via the platform 3516. Accordingly, in an interconnected device embodiment, implementation of functionality described herein may be distributed throughout the system 3500. For example, the functionality may be implemented in part on the computing device 3502 as well as via the platform 3516 that abstracts the functionality of the cloud 3514.

In addition to the embodiments expressly described herein, it is to be understood that all of the features disclosed in this disclosure may be combined in any combination (e.g., permutation, combination). Each element disclosed in the disclosure may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the characteristics of the present invention, and without departing from the spirit and scope thereof, and can make various changes and modifications of the invention to adapt it to various usages and conditions. Thus, other embodiments are also within the claims.

### EQUIVALENTS AND SCOPE

The articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that are dependent on the same base claim. Where elements are presented as lists, e.g., in Markush group format, each subgroup of the elements is also disclosed, and any element(s) can be removed from the group. It should be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permit the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description but rather is as set forth in the appended embodiments. Those of ordinary skill in the art will appreciate that various changes and modifications to this description may be made without departing from the spirit or scope of the present invention, as defined in the following embodiments.

### EMBODIMENTS

Features described above as well as those claimed below may be combined in various ways without departing from the scope hereof. The following examples illustrate some possible, non-limiting combinations:

The disclosure also provides support for a system comprising: a classification module implemented in a non-transitory computer-readable storage medium and configured to: classify a sample as positive or negative for circulating tumor DNA based on a dynamic probability score relative to a threshold, the dynamic probability score determined from targeted cell-free DNA sequencing data of the sample using specificity factors, the specificity factors including a number of mutated duplexes in the sample bearing mutations assayed in the sample and a total number of assumed duplexes assayed for mutations in the sample, the dynamic probability score further based on: a first likelihood that the number of mutated duplexes is exclusively derived from tumors, and a second likelihood that the number of mutated duplexes is spontaneous error or mutations that are not cancer derived, and output the classification of the sample. In a first example of the system, the targeted cell-free DNA sequencing data is enriched for DNA duplexes with mutation sites found in a tumor fingerprint. In a second example of the system, optionally including the first example, the tumor fingerprint is associated with a patient from which the sample is obtained. In a third example of the system, optionally including one or both of the first and second examples, the targeted cell-free DNA sequencing data is further enriched for mutated DNA duplexes with additional mutation sites found in tumor fingerprints associated with other patients. In a fourth example of the system, optionally including one or more or each of the first through third examples, the tumor fingerprint is associated with a patient other than the patient from which the sample is obtained. In a fifth example of the system, optionally including one or more or each of the first through fourth examples, the targeted cell-free DNA sequencing data is enriched for the DNA duplexes using MAESTRO enrichment. In a sixth example of the system, optionally including one or more or each of the first through fifth examples, the dynamic probability score is further based on a background mutation frequency. In a seventh example of the system, optionally including one or more or each of the first through sixth examples, the background mutation frequency is a fixed value, a context-specific value, or a sample-specific value. In an eighth example of the system, optionally including one or more or each of the first through seventh examples, the first likelihood and the second likelihood are binomial likelihoods. In a ninth example of the system, optionally including one or more or each of the first through eighth examples, the first likelihood is a binomial likelihood, and the second likelihood is a beta-binomial likelihood. In a tenth example of the system, optionally including one or more or each of the first through ninth examples, the system further comprises: a quantification and analysis module implemented in the non-transitory computer-readable storage medium and configured to: determine the number of mutated duplexes in the sample bearing the mutations assayed in the sample, and estimate the total number of assumed duplexes assayed for the mutations in the sample. In an eleventh example of the system, optionally including one or more or each of the first through tenth examples, the total number of assumed duplexes assayed for the mutations in the sample is estimated using a pre-determined number of least enriched sites of the mutations. In a twelfth example of the system, optionally including one or more or each of the first through eleventh examples, the total number of assumed duplexes assayed for the mutations in the sample is estimated using control probes designed without bias for mutated alleles versus wild-type alleles. In a thirteenth example of the system, optionally including one or more or each of the first through twelfth examples, the classification module is further configured to: output the first likelihood via a first likelihood model that uses the number of mutated duplexes in the sample and the total number of assumed duplexes assayed for the mutations in the sample, and output the second likelihood via a second likelihood model that uses the number of mutated duplexes in the sample and the total number of assumed duplexes assayed for the mutations in the sample. In a fourteenth example of the system, optionally including one or more or each of the first through thirteenth examples, the first likelihood model comprises a binomial distribution that models the number of mutated duplexes in the sample and the total number of assumed duplexes assayed for the mutations in the sample given a probability parameter based on an assumed background mutation frequency. In a fifteenth example of the system, optionally including one or more or each of the first through fourteenth examples, the second likelihood model comprises a binomial distribution that models the number of mutated duplexes in the sample and the total number of assumed duplexes assayed for the mutations in the sample given an assumed background mutation frequency. In a sixteenth example of the system, optionally including one or more or each of the first through fifteenth examples, the specificity factors further comprise mutation context-specific factors including a background mutation frequency that is adjusted for a plurality of mutation contexts. In a seventeenth example of the system, optionally including one or more or each of the first through sixteenth examples, the classification module is further configured to: output the first likelihood via a first likelihood model that uses the number of mutated duplexes in the sample for a given mutation context of the plurality of mutation contexts and the total number of assumed duplexes assayed for the mutations in the sample for the given mutation context, the first likelihood corresponding to a product of individual first likelihoods for the plurality of mutation contexts, and output the second likelihood via a second likelihood model that uses the number of mutated duplexes in the sample for the given mutation context of the plurality of mutation contexts, the total number of assumed duplexes assayed for the mutations in the sample for the given mutation context, an observed background mutation frequency in the sample for the given mutation context, and a number of observed background bases in the sample for the given mutation context, the second likelihood corresponding to a product of individual second likelihoods for the plurality of mutation contexts. In an eighteenth example of the system, optionally including one or more or each of the first through seventeenth examples, the first likelihood model is a binomial model. In a nineteenth example of the system, optionally including one or more or each of the first through eighteenth examples, the second likelihood model is a beta-binomial model. In a twentieth example of the system, optionally including one or more or each of the first through nineteenth examples, the classification module is further configured to: output the dynamic probability score via a Bayesian probability calculation that uses the first likelihood and the second likelihood. In a twenty-first example of the system, optionally including one or more or each of the first through twentieth examples, to classify the sample as positive or negative for the circulating tumor DNA based on the dynamic probability score relative to the threshold, the classification module is further configured to: classify the sample as positive for the circulating tumor DNA in response to the dynamic probability score being greater than or equal to the threshold, and classify the sample as negative for the circulating tumor DNA in response to the dynamic probability score being less than the threshold.

The disclosure also provides support for a method comprising: receiving targeted cell-free DNA sequencing data for a sample from a patient, the targeted cell-free DNA sequencing data enriched for DNA duplexes with mutation sites found in a tumor fingerprint, determining a dynamic probability score for a minimal residual disease (MRD) status of the sample based on specificity factors including a number of mutated duplexes in the sample and a total number of assumed duplexes assayed for the mutation sites in the sample by: outputting, via a first likelihood model that uses the number of mutated duplexes and the total number of assumed duplexes assayed for the mutation sites in the sample, a first likelihood that the number of mutated duplexes is exclusively derived from tumors, outputting, via a second likelihood model that uses the number of mutated duplexes and the total number of assumed duplexes assayed for the mutation sites in the sample, a second likelihood that the number of mutated duplexes is spontaneous error or mutations that are not cancer derived, and determining the dynamic probability score based on the first likelihood and the second likelihood, classifying the MRD status of the sample as MRD positive or MRD negative based on the dynamic probability score relative to a threshold, and outputting the classified MRD status. In a first example of the method, classifying the MRD status of the sample as MRD positive or MRD negative based on the dynamic probability score relative to the threshold comprises: classifying the MRD status of the sample as MRD positive in response to the dynamic probability score being greater than or equal to the threshold, or classifying the MRD status of the sample as MRD negative in response to the dynamic probability score being less than the threshold. In a second example of the method, optionally including the first example, the specificity factors further comprise mutation context-specific factors. In a third example of the method, optionally including one or both of the first and second examples, the mutation context-specific factors comprise an observed background mutation frequency of the sample and a number of observed background bases in the sample for respective mutation contexts of a plurality of mutation contexts for the mutation sites. In a fourth example of the method, optionally including one or more or each of the first through third examples, the method further comprises: adjusting parameters of the first likelihood model and the second likelihood model based on the mutation context-specific factors. In a fifth example of the method, optionally including one or more or each of the first through fourth examples, the first likelihood model and the second likelihood model further use a background mutation frequency. In a sixth example of the method, optionally including one or more or each of the first through fifth examples, the background mutation frequency is a fixed value, a context-specific value, or a sample-specific value. In a seventh example of the method, optionally including one or more or each of the first through sixth examples, the targeted cell-free DNA sequencing data is further enriched for additional DNA duplexes with additional mutation sites found in additional tumor fingerprints. In an eighth example of the method, optionally including one or more or each of the first through seventh examples, the first likelihood model and the second likelihood model are binomial models. In a ninth example of the method, optionally including one or more or each of the first through eighth examples, the first likelihood model is a binomial model, and the second likelihood model is a beta-binomial model. In a tenth example of the method, optionally including one or more or each of the first through ninth examples, determining the dynamic probability score based on the first likelihood and the second likelihood comprises using the first likelihood and the second likelihood in a Bayesian probability calculation.

(A1) A method for determining whether a patient sample obtained from a patient is positive for minimal residual disease (MRD) using a targeted sequencing assay for mutations in a set of patient-specific tumor mutations, the method comprising:
using at least one computer hardware processor to perform:
   obtaining sequencing data for the patient, the sequencing data having been previously obtained by sequencing the patient sample using probes of the targeted sequencing assay that are designed to enrich for DNA duplexes with mutations in the set of patient-specific tumor mutations;
   estimating, using the sequencing data,
      (i) a number of mutated DNA duplexes in the patient sample, and
      (ii) a total number of DNA duplexes assayed for mutations in the patient sample;
   determining a posterior probability that the patient sample is positive for MRD using the number of mutated DNA duplexes and the total number of DNA duplexes assayed for mutations in the patient sample, the determining comprising:
      determining, using a first statistical model, the number of mutated DNA duplexes and the total number of DNA duplexes, a first likelihood of the number of mutated DNA duplexes, from among the total number of DNA duplexes, being exclusively tumor derived;
      determining, using a second statistical model, the number of mutated DNA duplexes and the total number of DNA duplexes, a second likelihood of the number of mutated DNA duplexes, from among the total number of DNA duplexes, being spontaneous errors or mutations that are not cancer derived; and
      determining the posterior probability that the patient sample is positive for MRD using the first likelihood and the second likelihood;
   when the determined posterior probability that the patient sample is positive for MRD is below a threshold, determining that the patient sample is negative for MRD; and
   when the determined posterior probability that the patient sample is positive for MRD exceeds the threshold, determining that the patient sample is positive for MRD.

(A2) A method for determining whether a patient sample obtained from a patient contains circulating tumor DNA (ctDNA) using a targeted sequencing assay for mutations in a set of patient-specific tumor mutations, the method comprising:
using at least one computer hardware processor to perform:
   obtaining sequencing data for the patient, the sequencing data having been previously obtained by sequencing the patient sample using probes of the targeted sequencing assay that are designed to enrich for DNA duplexes with mutations in the set of patient-specific tumor mutations;
   estimating, using the sequencing data,
      (i) a number of mutated DNA duplexes in the patient sample, and
      (ii) a total number of DNA duplexes assayed for mutations in the patient sample;
   determining a posterior probability that the patient sample is positive for MRD using the number of mutated DNA duplexes and the total number of DNA duplexes assayed for mutations in the patient sample, the determining comprising:
      determining, using a first statistical model, the number of mutated DNA duplexes and the total number of DNA duplexes, a first likelihood of observing the number of mutated DNA duplexes, from among the total number of DNA duplexes, assuming the patient sample contains ctDNA;
      determining, using a second statistical model, the number of mutated DNA duplexes and the total number of DNA duplexes and estimated error profiles, a second likelihood of the number of mutant DNA duplexes, from among the total number of DNA duplexes, assuming the patient sample does not contain ctDNA; and
      determining the posterior probability that the patient sample contains ctDNA using the first likelihood and the second likelihood;
   determining that patient sample does not contain ctDNA when the posterior probability is below a threshold; and
   determining that patient sample contains ctDNA when the posterior probability exceeds the threshold.

(A3) For the method denoted as (A1) or (A2), wherein the sequencing is performed using the targeted sequencing assay and comprises:
(i) obtaining DNA duplexes;
(ii) attaching a unique molecular identifier (UMI) to 5′ and 3′ ends of the DNA duplexes to produce tagged duplexes, wherein each UMI is unique to each tagged duplex;
(iii) amplifying the tagged duplexes by polymerase chain reaction (PCR) to produce amplified duplexes;
(iv) denaturing the tagged duplexes to produce single-stranded amplified DNA;
(v) capturing single-stranded amplified DNA having one mutation in the set of patient-specific tumor mutations using an allele-specific probe that anneals to one mutation in the set of patient-specific tumor mutations to produce an enriched sample;
(vi) sequencing the enriched sample; and
(vii) identifying the presence of mutated DNA duplexes if one mutation in the set of patient-specific tumor mutations is observed in both strands of the tagged duplexes as identified by UMIs.

(A4) For the method denoted as (A3), wherein a plurality of allele-specific probes, each specific to different mutation sites, are used to anneal to one or more of mutation sites in the set of patient-specific tumor mutations to produce the enriched sample.

(A5) For the method denoted as (A4), wherein each of the plurality of allele-specific probes are specific to mutation sites found in sets of patient-specific tumor mutations derived from respective different patients, or wherein the plurality of allele-specific probes each derived from different patients are pooled and applied to a single patient sample.

(A6) For the method denoted as (A1) or (A2), wherein estimating the total number of DNA duplexes assayed for mutations in the patient sample is performed using probes that have limited selectivity for mutated versus non-mutated DNA.

(A7) For the method denoted as (A1) or (A2), wherein estimating the total number of DNA duplexes assayed for mutations in the patient sample is performed using a threshold number of least-enriched sites.

(A8) For the method denoted as (A7), wherein the least-enriched sites contain mutated and non-mutated DNA duplexes captured by the probes.

(A9) For the method denoted as (A1) or (A2), wherein estimating the total number of DNA duplexes assayed for mutations in the patient sample is performed using a subset of control probes that do not have specificity for the patient-specific tumor mutations and determining a value of mutated and wildtype DNA duplexes based on sequencing the subset of control probes.

(A10) For the method denoted as (A9), further comprising multiplying a number of the patient-specific tumor mutations assayed by a value indicative of an average or median value of mutated and non-mutated DNA duplexes at a subset of sites to estimate the total number of DNA duplexes assayed for mutations in the patient sample.

(A11) For the method denoted as any one of (A1)-(A10), wherein posterior probability that the patient sample is positive for MRD is determined according to:

$$P ( {M}^{+} \vert D ) = \frac{P ( D \vert {M}^{+} ) P ( {M}^{+} )}{{\sum }_{i ∈ \{ + , - \}} P ( D \vert {M}^{i} ) P ( {M}^{i} )}$$

wherein P(D|M⁺) is the first likelihood and P (D|M⁻) is the second likelihood,
wherein M+ indicates MRD positive status and P(M⁺) indicates its prior probability,
wherein M− indicates MRD negative status and P(M⁻) indicates its prior probability, and
wherein D indicates the sequencing data.

(A12) For the method denoted as any one of (A1)-(A10), wherein the first statistical model is a Binomial model.

(A13) For the method denoted as any one of (A1)-(A10), wherein the first likelihood is determined as:

P(D|M⁺)=Binomial(#ALT duplexes,#total duplexes,t)
wherein #ALT duplexes is the number of mutated DNA duplexes in the patient sample,
wherein #total duplexes is the total number of DNA duplexes assayed for mutations in the patient sample,
wherein t=max ((#ALT)/(#total), a background mutation frequency),
wherein M+ indicates MRD positive status and D indicates the sequencing data.

(A14) For the method denoted as (A13), further comprising setting the background mutation frequency to:
(a) a same value for all mutations;
(b) different background mutation frequencies for mutations in different mutation contexts; or
(c) a value empirically measured on a per sample basis or different values empirically measured for different mutation contexts.

(A15) For the method denoted as (A14), wherein when the background mutation frequency is set to a value empirically measured on a per sample basis or different values empirically measured for different mutation contexts, and the method further comprises using pooled probe testing to determine the value or values empirically or using targeted duplex sequencing on the patient sample to determine the value or values empirically.

(A16) For the method denoted as any one of (A1)-(A12), wherein the first likelihood is determined as a product of context-specific and sample specific likelihoods for a discrete set of mutation contexts.

(A17) For the method denoted as (A15), wherein the first likelihood is determined as:

$$P ( D \vert {M}^{+} ) = {{\prod }_{}}_{c} Binomial ( \# ALT {duplexes}_{c} , \# total {duplexes}_{c} , \frac{{TF}_{x}}{2} )$$

for c∈{C>G, T>A,T>C,T>G},
wherein {C>G,T>A,T>C,T>G} is the discrete set of mutation contexts,
wherein c represents a mutation context in the discrete set of mutation contexts,
wherein #ALT duplexes, is the number of mutated DNA duplexes in the patient sample in mutation context c,
wherein #total duplexes, is a total number of DNA duplexes assayed for mutations in mutation context c, and
wherein TFx is tumor fraction estimated for the patient sample.

(A18) For the method denoted as any one of (A1)-(A17), wherein the second statistical model is a Binomial model.

(A19) For the method denoted as (A18), wherein the second likelihood is determined as:

P(D|M⁻)=Binomial(#ALT duplexes,#total duplexes,error rate)
wherein #ALT duplexes is the number of mutated DNA duplexes in the patient sample,
wherein #total duplexes is the total number of DNA duplexes assayed for mutations in the patient sample,
wherein error rate is a background mutation frequency, and
wherein M⁻ indicates MRD negative status and D indicates the sequencing data.

(A20) For the method denoted as (A19), further comprising setting the background mutation frequency to:
(a) a same value for all mutations;
(b) different background mutation frequencies for mutations in different mutation contexts; or
(c) a value empirically measured on a per sample basis or different values empirically measured for different mutation contexts.

(A21) For the method denoted as (A20), wherein when the background mutation frequency is set to a value empirically measured on a per sample basis or different values empirically measured for different mutation contexts, and the method further comprises using pooled probe testing to determine the value or values empirically or using targeted duplex sequencing in the patient sample to determine the value or values empirically.

(A22) For the method denoted as any one of (A1)-(A12), wherein the second likelihood is determined as a product of context-specific likelihoods for a discrete set of mutation contexts to provide for context-specific noise tuning.

(A23) For the method denoted as (A1) or (A2), wherein the second statistical model is a Beta-Binomial model.

(A24) For the method denoted as (A22) or (A23), wherein the second likelihood is determined as a product of Beta-Binomial likelihoods, determined for respective contexts using Beta-Binomial models with context-specific priors set based on context-specific background mutation frequencies.

(A25) For the method denoted as (A24), wherein the context-specific background mutation frequencies are determined using sample-specific SNV frequency measured by the targeted sequencing assay.

(A26) For the method denoted as any one of (A22)-(A24), wherein the second likelihood is determined as:

$$P ( D \vert {M}^{-} ) = {{\prod }_{}}_{c} Beta Binomial ( ALT {duplexes}_{c} , total {duplexes}_{c} , {α}_{c} , {β}_{c} )$$

for c∈{C>G, T>A,T>C, T>G}
wherein BetaBinom represents Beta-Binomial model,
wherein {C>G,T>A,T>C,T>G} is the discrete set of mutation contexts,
wherein c represents a mutation context in the discrete set of mutation contexts,
wherein #ALT duplexes, is number of mutated DNA duplexes in the patient sample in mutation context c,
wherein #total duplexes_{c} is a total number of DNA duplexes assayed for mutations in mutation context c,
wherein α_{c}=αᵢ+ [SNVs Observed, from SNV frequency estimation],
wherein

$${α}_{i} = ( \frac{1 - {μ}_{i}}{{σ}_{i}^{2}} - \frac{1}{{μ}_{i}} ) {μ}_{i}^{2} ,$$

and, optionally, μᵢ=0.05 ppm, σᵢ=0.05 ppm,
wherein β_{c}=βᵢ+[Bases Sequenced_{c} from SNV frequency estimation]−[SNVs Observed_{c} from SNV frequency estimation], and
wherein

$${β}_{i} = {α}_{i} ( \frac{1}{μ} - 1 ) .$$

(A27) For the method denoted as any one of (A1)-(A26), wherein the set of patient-specific tumor mutations is determined by:
(i) sequencing tumor genomic DNA from a tumor derived from the patient to identify a plurality of mutation sites in the tumor genomic DNA; and
(ii) processing and filtering the plurality of mutation sites to identify at least one selected mutation site to associate with the patient.

(A28) For the method denoted as (A27), wherein processing and filtering the plurality of mutation sites comprises:
selecting at least one of the plurality of mutation sites;
analyzing the at least one selected mutation site in matched normal genomic DNA;
analyzing the at least one selected mutation site in matched tumor genomic DNA;
determining for the at least one selected mutation site, a first number of mutated duplexes in the matched normal genomic DNA;
determining for the at least one selected mutation site, a second number of mutated duplexes in the matched tumor genomic DNA;
determining a ratio of mutated duplexes to mutated single strand consensus molecules in the matched tumor genomic DNA; and
determining whether the first number of mutated duplexes in the matched normal genomic DNA is zero, the second number of mutated duplexes in the matched tumor genomic DNA is greater than zero, and the ratio of mutated duplexes to mutated single strand consensus molecules in the matched tumor genomic DNA is greater than 0.15.

(A29) For the method denoted as (A28), wherein if the first number of mutated duplexes in the matched normal genomic DNA is zero, the second number of mutated duplexes in the matched tumor genomic DNA is greater than zero, and the ratio of mutated duplexes to mutated single strand consensus molecules in the matched tumor genomic DNA is greater than 0.15, then identifying the at least one selected mutation site for consideration of minimal residual disease detection.

(A30) For the method denoted as (A28), wherein if the first number of mutated duplexes in the matched normal genomic DNA is not zero, the second number of mutated duplexes in the matched tumor genomic DNA is not greater than zero, and the ratio of mutated duplexes to mutated single strand consensus molecules in the matched tumor genomic DNA is not greater than 0.15, then not identifying the at least one selected mutation site for consideration of minimal residual disease detection, and then checking a plurality of criteria to determine whether detection is impacted by germline or somatic factors.

(A31) For the method denoted as (A30), wherein the plurality of criteria comprise one or more of passed tumor validation, the at least one selected mutation site is not in a matched set of patient-specific tumor mutations of the patient sample, the first number of mutated duplexes in the matched normal genomic DNA is zero, the second number of mutated duplexes in the matched tumor genomic DNA is zero, and the ratio of mutated duplexes to mutated single strand consensus molecules in the matched tumor genomic DNA is greater than 0.15.

(A32) For the method denoted as any one of (A1)-(A31), wherein the patient sample is a biological sample.

(A33) For the method denoted as (A32), wherein the biological sample is a blood sample.

(A34) For the method denoted as any one of (A1)-(A31), wherein the patient sample is derived from a liquid biopsy.

(A35) For the method denoted as any one of (A1)-(A34), wherein the patient has cancer or had cancer.

(A36) For the method denoted as (A35), wherein the cancer is blood or lymphoid cancer, a bone or soft tissue cancer, a brain or central nervous system cancer, breast cancer, a childhood cancer, a digestive system cancer, an eye cancer, a head or neck cancer, lung cancer, a pelvic area cancer, a skin cancer, or a urinary cancer.

(A37) For the method denoted as (A35) or (A36), wherein the cancer is glioblastoma.

(A38) For the method denoted as (A35) or (A36), wherein the cancer is melanoma.

(A39) For the method denoted as any one of (A1)-(A38), wherein the sequencing is next-generation sequencing (NGS).

(A40) For the method denoted as (A1) or (A2), further comprising:
prior to determining the posterior probability that the patient sample is positive for MRD, treating the patient according to an initial treatment plan;
in response to determining that the patient sample is positive for MRD, altering the initial treatment plan to create an updated treatment plan; and
treating the patient according to the updated treatment plan.

(A41) For the method denoted as (A40), wherein:
the initial treatment plan comprises administering a first drug at a first dosage to the patient,
altering the initial treatment plan comprises increasing the first dosage to a second dosage, and
the updated treatment plan comprises administering the first drug at the second dosage to the patient.

(A42) For the method denoted as (A40), wherein:
the initial treatment plan includes performing surgery on the patient;
altering the treatment plan comprises determining that further therapy is needed; and
the updated treatment plan comprises administering the further therapy to the patient.

(A43) For the method denoted as (A42), wherein the further therapy comprises adjuvant chemotherapy.

(A44) For the method denoted as (A40), wherein:
the initial treatment plan comprises administering a first drug to the patient and/or performing surgery on the patient,
altering the initial treatment plan comprises determining that a second drug is to be administered to the patient, and
the updated treatment plan comprises administering the second drug to the patient.

(A45) For the method denoted as (A1) or (A2), further comprising:
prior to determining the posterior probability that the patient sample is negative for MRD, treating the patient according to an initial treatment plan;
in response to determining that the patient sample is negative for MRD, altering the initial treatment plan to create an updated treatment plan; and
treating the patient according to the updated treatment plan.

(A46) For the method denoted as (A45), wherein:
the initial treatment plan comprises administering a first drug at a first dosage to the patient,
altering the initial treatment plan comprises decreasing the first dosage to a second dosage, and
the updated treatment plan comprises administering the first drug at the second dosage to the patient.

(A47) For the method denoted as (A45), wherein:
the initial treatment plan comprises administering a first drug to the patient,
altering the initial treatment plan comprises eliminating the first drug from the initial treatment plan, and
the updated treatment plan comprises treating the patient without administering the first drug to the patient or stopping treatment of the patient.

(A48) For the method denoted as any one of (A1)-(A47), wherein MRD is detected at between at least 0.10 and at least 10.0 parts per million (ppm) of tumor-derived cell-free DNA.

(A49) For the method denoted as (A48), wherein MRD is detected at at least 0.10, at least 0.15, at least 0.20, at least 0.25, at least 0.30, at least 0.35, at least 0.40, at least 0.45, at least 0.50, at least 0.55, at least 0.60, at least 0.65, at least 0.70, at least 0.75, at least 0.80, at least 0.85, at least 0.90, at least 0.95, or at least 1.00 ppm.

(A50) For the method denoted as any one of (A1)-(A49), wherein MRD is detected with at least 95%, at least 96%, at least 97%, at least 98%, at least 99%, or 100% specificity.

(B1) A system, comprising:
at least one computer hardware processor; and
at least one non-transitory computer-readable storage medium storing processor executable instructions that, when executed by the at least one computer hardware processor, cause the at least one computer hardware processor to perform the method denoted as any one of (A1)-(A39) or (A48)-(A50).

(C1) At least one non-transitory computer-readable storage medium storing processor executable instructions that, when executed by at least one computer hardware processor, cause the at least one computer hardware processor to perform the method denoted as any one of (A1)-(A39) or (A48)-(A50).

1. A system comprising:
a classification module implemented in a non-transitory computer-readable storage medium and configured to:
classify a sample as positive or negative for circulating tumor DNA based on a dynamic probability score relative to a threshold, the dynamic probability score determined from targeted cell-free DNA sequencing data of the sample using specificity factors, the specificity factors including a number of mutated duplexes in the sample bearing mutations assayed in the sample and a total number of assumed duplexes assayed for mutations in the sample, the dynamic probability score further based on:
a first likelihood that the number of mutated duplexes is exclusively derived from tumors; and
a second likelihood that the number of mutated duplexes [are] is spontaneous error or mutations that are not cancer derived; and
output the classification of the sample.

2. The system of claim 1, wherein the targeted cell-free DNA sequencing data is enriched for DNA duplexes with mutation sites found in a tumor fingerprint.

3. The system of claim 2, wherein the tumor fingerprint is associated with a patient from which the sample is obtained.

4. The system of claim 3, wherein the targeted cell-free DNA sequencing data is further enriched for mutated DNA duplexes with additional mutation sites found in tumor fingerprints associated with other patients.

5. The system of claim 2, wherein the tumor fingerprint is associated with a patient other than the patient from which the sample is obtained.

6. The system of claim 2, wherein the targeted cell-free DNA sequencing data is enriched for the DNA duplexes using MAESTRO enrichment.

7. The system of claim 1, wherein the dynamic probability score is further based on a background mutation frequency.

8. The system of claim 7, wherein the background mutation frequency is a fixed value, a context-specific value, or a sample-specific value.

9. (canceled)

10. The system of claim 1, wherein the first likelihood is a binomial likelihood, and the second likelihood is a binomial likelihood or a beta-binomial likelihood.

11. The system of claim 1, further comprising a quantification and analysis module implemented in the non-transitory computer-readable storage medium and configured to:
determine the number of mutated duplexes in the sample bearing the mutations assayed in the sample; and
estimate the total number of assumed duplexes assayed for the mutations in the sample.

12. The system of claim 11, wherein the total number of assumed duplexes assayed for the mutations in the sample is estimated using a pre-determined number of least enriched sites of the mutations or using control probes designed without bias for mutated alleles versus wild-type alleles.

13. (canceled)

14. The system of claim 1, wherein the classification module is further configured to:
output the first likelihood via a first likelihood model that uses the number of mutated duplexes in the sample and the total number of assumed duplexes assayed for the mutations in the sample; and
output the second likelihood via a second likelihood model that uses the number of mutated duplexes in the sample and the total number of assumed duplexes assayed for the mutations in the sample.

15. The system of claim 14, wherein the first likelihood model and/or the second likelihood model comprises a binomial distribution that models the number of mutated duplexes in the sample and the total number of assumed duplexes assayed for the mutations in the sample given a probability parameter based on an assumed background mutation frequency.

16. (canceled)

17. The system of claim 1, wherein the specificity factors further comprise mutation context-specific factors including a background mutation frequency that is adjusted for a plurality of mutation contexts.

18. The system of claim 17, wherein the classification module is further configured to:
output the first likelihood via a first likelihood model that uses the number of mutated duplexes in the sample for a given mutation context of the plurality of mutation contexts and the total number of assumed duplexes assayed for the mutations in the sample for the given mutation context, the first likelihood corresponding to a product of individual first likelihoods for the plurality of mutation contexts; and
output the second likelihood via a second likelihood model that uses the number of mutated duplexes in the sample for the given mutation context of the plurality of mutation contexts, the total number of assumed duplexes assayed for the mutations in the sample for the given mutation context, an observed background mutation frequency in the sample for the given mutation context, and a number of observed background bases in the sample for the given mutation context, the second likelihood corresponding to a product of individual second likelihoods for the plurality of mutation contexts.

19. The system of claim 18, wherein the first likelihood model is a binomial model and/or the second likelihood model is a beta-binomial model.

20. (canceled)

21. The system of claim 1, wherein the classification module is further configured to:
output the dynamic probability score via a Bayesian probability calculation that uses the first likelihood and the second likelihood.

22. The system of claim 1, wherein to classify the sample as positive or negative for the circulating tumor DNA based on the dynamic probability score relative to the threshold, the classification module is further configured to:
classify the sample as positive for the circulating tumor DNA in response to the dynamic probability score being greater than or equal to the threshold; and
classify the sample as negative for the circulating tumor DNA in response to the dynamic probability score being less than the threshold.

23. A method comprising:
receiving targeted cell-free DNA sequencing data for a sample from a patient, the targeted cell-free DNA sequencing data enriched for DNA duplexes with mutation sites found in a tumor fingerprint;
determining a dynamic probability score for a minimal residual disease (MRD) status of the sample based on specificity factors including a number of mutated duplexes in the sample and a total number of assumed duplexes assayed for the mutation sites in the sample by:
outputting, via a first likelihood model that uses the number of mutated duplexes and the total number of assumed duplexes assayed for the mutation sites in the sample, a first likelihood that the number of mutated duplexes is exclusively derived from tumors;
outputting, via a second likelihood model that uses the number of mutated duplexes and the total number of assumed duplexes assayed for the mutation sites in the sample, a second likelihood that the number of mutated duplexes is spontaneous error or mutations that are not cancer derived; and
determining the dynamic probability score based on the first likelihood and the second likelihood;
classifying the MRD status of the sample as MRD positive or MRD negative based on the dynamic probability score relative to a threshold; and
outputting the classified MRD status.

24. The method of claim 23, wherein classifying the MRD status of the sample as MRD positive or MRD negative based on the dynamic probability score relative to the threshold comprises:
classifying the MRD status of the sample as MRD positive in response to the dynamic probability score being greater than or equal to the threshold; or
classifying the MRD status of the sample as MRD negative in response to the dynamic probability score being less than the threshold.

25. (canceled)

26. The method of claim 23, wherein the specificity factors further comprise mutation context-specific factors, which comprise an observed background mutation frequency of the sample and a number of observed background bases in the sample for respective mutation contexts of a plurality of mutation contexts for the mutation sites.

27. The method of claim 26, further comprising:
adjusting parameters of the first likelihood model and the second likelihood model based on the mutation context-specific factors.

28. The method of claim 23, wherein the first likelihood model and the second likelihood model further use a background mutation frequency.

29. The method of claim 28, wherein the background mutation frequency is a fixed value, a context-specific value, or a sample-specific value.

30. The method of claim 23, wherein the targeted cell-free DNA sequencing data is further enriched for additional DNA duplexes with additional mutation sites found in additional tumor fingerprints.

31. (canceled)

32. The method of claim 23, wherein the first likelihood model is a binomial model, and the second likelihood model is a binomial model or a beta-binomial model.

33. The method of claim 23, wherein determining the dynamic probability score based on the first likelihood and the second likelihood comprises using the first likelihood and the second likelihood in a Bayesian probability calculation.
